SEQUENCE LISTING (170 sequences)

SEQ ID NO: 1 - protein, 26 aa, synthetic construct
  REGION 1..26: note = FR1
  SITE 1: note = X is Q, E, or D
  SITE 5: note = X is V, Q, A, or E
  SITE 7: note = X is S or T
  SITE 11: note = X is L, S, or V
  SITE 12: note = X is V or A
  SITE 14: note = X is P, A, or T
  SITE 16: note = X is G, D, or R
  SITE 23: note = X is A, V, T, or E
  SITE 24: note = X is A or V
XVQLXEXGGGXXQXGXSLRLSCXXSG

SEQ ID NO: 2 - protein, 18 aa, synthetic construct
  REGION 1..18: note = FR2
  SITE 1: note = X is M, I, V, or L
  SITE 2: note = X is G, S, or A
  SITE 4: note = X is F, Y, or V
  SITE 7: note = X is A, V, P, or T
  SITE 11: note = X is E, G, A, or Q
  SITE 12: note = X is R or L
  SITE 14: note = X is F, G, W, or L
  SITE 16: note = X is A, G, or S
  SITE 17: note = X is A, S, or G
XXWXRQXPGKXXEXVXXI

SEQ ID NO: 3 - protein, 39 aa, synthetic construct
  REGION 1..39: note = FR3
  SITE 2: note = X is A, G, S, or T
  SITE 13: note = X is R or Q
  SITE 15: note = X is N, S, or D
  SITE 16: note = X is S, A, or D
  SITE 18: note = X is N or K
  SITE 19: note = X is T or M
  SITE 21: note = X is Y, D, or S
  SITE 25: note = X is N or D
  SITE 26: note = X is S or N
  SITE 29: note = X is P or A
YXDSVKGRFTISXDXXKXXVXLQMXXLRXEDTAXYYCAA

SEQ ID NO: 4 - protein, 12 aa, synthetic construct
  REGION 1..12: note = FR4
  SITE 4: note = X is Q or K
  SITE 7: note = X is L or Q
YWGXGTXVTVSS

SEQ ID NO: 5 - protein, 26 aa, synthetic construct
  REGION 1..26: note = framework region 1
QVQLVESGGGLVQPGGSLRLSCAASG

SEQ ID NO: 6 - protein, 18 aa, synthetic construct
  REGION 1..18: note = framework region 2
MGWFRQAPGKEREFVAAI

SEQ ID NO: 7 - protein, 39 aa, synthetic construct
  REGION 1..39: note = framework region 3
YADSVKGRFTISRDNSKNTVYLQMNSLRPEDTAVYYCAA

SEQ ID NO: 8 - protein, 12 aa, synthetic construct
  REGION 1..12: note = framework region 4
YWGQGTLVTVSS

SEQ ID NO: 9 - protein, 7 aa, synthetic construct
  REGION 1..7: note = Nb42 CDR1
FAYSTYS

SEQ ID NO: 10 - protein, 8 aa, synthetic construct
  REGION 1..8: note = Nb42 CDR2
NSGTFRLW

SEQ ID NO: 11 - protein, 16 aa, synthetic construct
  REGION 1..16: note = Nb42 CDR3
RAWSPYSSTVDAGDFR

SEQ ID NO: 12 - protein, 7 aa, synthetic construct
  REGION 1..7: note = 2H10 CDR1
RRFSIEA

SEQ ID NO: 13 - protein, 7 aa, synthetic construct
  REGION 1..7: note = 2H10 CDR2
DSGGSTD

SEQ ID NO: 14 - protein, 11 aa, synthetic construct
  REGION 1..11: note = 2H10 CDR3
IGSSWYGRGLD

SEQ ID NO: 15 - protein, 7 aa, synthetic construct
  REGION 1..7: note = aTNFa-mu CDR1
GTFSSII

SEQ ID NO: 16 - protein, 8 aa, synthetic construct
  REGION 1..8: note = aTNFa-mu CDR2
SWSGGTTV

SEQ ID NO: 17 - protein, 15 aa, synthetic construct
  REGION 1..15: note = aTNFa-mu CDR3
RPYQKYNWASASYNV

SEQ ID NO: 18 - protein, 7 aa, synthetic construct
  REGION 1..7: note = E1-1 CDR1
GGSDAGT

SEQ ID NO: 19 - protein, 8 aa, synthetic construct
  REGION 1..8: note = E1-1 CDR2
SWAGTAWR

SEQ ID NO: 20 - protein, 9 aa, synthetic construct
  REGION 1..9: note = E1-1 CDR3
LGSYEMDHH

SEQ ID NO: 21 - protein, 19 aa, synthetic construct
  REGION 1..19: note = aH linker
AEAAAKEAAAKEAAAKAGC

SEQ ID NO: 22 - protein, 21 aa, synthetic construct
  REGION 1..21: note = AE3K2R(2) linker
AEEEKRKAEEEKRKAEEEAGC

SEQ ID NO: 23 - protein, 28 aa, synthetic construct
  REGION 1..28: note = AE3K2R(3) linker
AEEEKRKAEEEKRKAEEEKRKAEEEAGC

SEQ ID NO: 24 - protein, 20 aa, synthetic construct
  REGION 1..20: note = E4K4(2) linker
AEEEEKKKKEEEEKKKKAGC

SEQ ID NO: 25 - protein, 14 aa, synthetic construct
  REGION 1..14: note = EA3K(2) linker
AEAAAKEAAAKAGC

SEQ ID NO: 26 - protein, 16 aa, synthetic construct
  REGION 1..16: note = Alfa linker
PSRLEEELRRRLTEGC

SEQ ID NO: 27 - protein, 77 aa, synthetic construct
  REGION 1..77: note = MyosinVI linker
AEEEEKKKQQEEEAERLRRIQEEMEKERKRREEDEERRRKEEEERRMKLEMEAKRKQEEEERKKREDDEKRKKKAGC

SEQ ID NO: 28 - protein, 13 aa, synthetic construct
  REGION 1..13: note = Spot linker
PDRVRAVSHWSSC

SEQ ID NO: 29 - protein, 20 aa, synthetic construct
  REGION 1..20: note = GT9 linker
GTGTGTGTGTGTGTGTGTGC

SEQ ID NO: 30 - protein, 17 aa, synthetic construct
  REGION 1..17: note = Modified Rigid linker
TPTTPPTPTPGTPPGGC

SEQ ID NO: 31 - protein, 17 aa, synthetic construct
  REGION 1..17: note = Rigid
SPSTPPTPSPSTPPGGC

SEQ ID NO: 32-50 - skipped (19 entries)

SEQ ID NO: 51 - protein, 120 aa, synthetic construct
  REGION 1..120: note = 2H10
QVQLVESGGGLVQAGGSLRLSCLASGRRFSIEAMGWYRQAPGKQRELVAIIDSGGSTDYVDSAKGRFTISRDNRKNTVDLQMNSLKDEDTGVYYCNVIGSSWYGRGLDYWGNGTQVTVSS

SEQ ID NO: 52 - protein, 120 aa, synthetic construct
  REGION 1..120: note = 2H10 N112Q
QVQLVESGGGLVQAGGSLRLSCLASGRRFSIEAMGWYRQAPGKQRELVAIIDSGGSTDYVDSAKGRFTISRDNRKNTVDLQMNSLKDEDTGVYYCNVIGSSWYGRGLDYWGQGTQVTVSS

SEQ ID NO: 53 - protein, 120 aa, synthetic construct
  REGION 1..120: note = 2H10 5MUT
QVQLVESGGGLVQAGGSLRLSCLASGRRFSIEAMGWYRQAPGKQRELVAIIDSGGSTDYVDSAKGRFTISRDNAKNTVYLQMNSLKPEDTAVYYCNVIGSSWYGRGLDYWGQGTQVTVSS

SEQ ID NO: 54 - protein, 120 aa, synthetic construct
  REGION 1..120: note = 2H10 6MUT
QVQLVESGGGLVQAGGSLRLSCLASGRRFSIEAMGWYRQAPGKQRELVAIIDSGGSTDYADSAKGRFTISRDNAKNTVYLQMNSLKPEDTAVYYCNVIGSSWYGRGLDYWGQGTQVTVSS

SEQ ID NO: 55 - protein, 120 aa, synthetic construct
  REGION 1..120: note = Hu2H10 5MUT
QVQLVESGGGLVQPGGSLRLSCLASGRRFSIEAMGWYRQAPGKQRELVAIIDSGGSTDYVDSAKGRFTISRDNSKNTVYLQMNSLRAEDTAVYYCNVIGSSWYGRGLDYWGQGTLVTVSS

SEQ ID NO: 56 - protein, 120 aa, synthetic construct
  REGION 1..120: note = Hu2H10 5MUT R86K A87P
QVQLVESGGGLVQPGGSLRLSCLASGRRFSIEAMGWYRQAPGKQRELVAIIDSGGSTDYVDSAKGRFTISRDNSKNTVYLQMNSLKPEDTAVYYCNVIGSSWYGRGLDYWGQGTLVTVSS

SEQ ID NO: 57 - protein, 120 aa, synthetic construct
  REGION 1..120: note = Hu2H10 5MUT L115Q
QVQLVESGGGLVQPGGSLRLSCLASGRRFSIEAMGWYRQAPGKQRELVAIIDSGGSTDYVDSAKGRFTISRDNSKNTVYLQMNSLRAEDTAVYYCNVIGSSWYGRGLDYWGQGTQVTVSS

SEQ ID NO: 58 - protein, 120 aa, synthetic construct
  REGION 1..120: note = Hu2H10 5MUT R86K A87P L115Q
QVQLVESGGGLVQPGGSLRLSCLASGRRFSIEAMGWYRQAPGKQRELVAIIDSGGSTDYVDSAKGRFTISRDNSKNTVYLQMNSLKPEDTAVYYCNVIGSSWYGRGLDYWGQGTQVTVSS

SEQ ID NO: 59-60 - skipped (2 entries)

SEQ ID NO: 61 - protein, 126 aa, synthetic construct
  REGION 1..126: note = Nb42
QVQLQESGGGSLQAGASLRLSCAASGFAYSTYSMGWFRQVSGKEREGVATINSGTFRLWYTDSVKGSFTISRDNAKNMLYLQMNSLKPEDTAIYYCAARAWSPYSSTVDAGDFRYWGQGTQVTVSS

SEQ ID NO: 62 - protein, 126 aa, synthetic construct
  REGION 1..126: note = HuNb42
QVQLVESGGGLVQPGGSLRLSCAASGFAYSTYSMGWFRQAPGKEREAVATINSGTFRLWYTDSVKGRFTISRDNSKNTLYLQMNSLRAEDTAVYYCAARAWSPYSSTVDAGDFRYWGQGTLVTVSS

SEQ ID NO: 63 - protein, 126 aa, synthetic construct
  REGION 1..126: note = HuNb42 P14A
QVQLVESGGGLVQAGGSLRLSCAASGFAYSTYSMGWFRQAPGKEREAVATINSGTFRLWYTDSVKGRFTISRDNSKNTLYLQMNSLRAEDTAVYYCAARAWSPYSSTVDAGDFRYWGQGTLVTVSS

SEQ ID NO: 64 - protein, 126 aa, synthetic construct
  REGION 1..126: note = HuNb42 T61A
QVQLVESGGGLVQPGGSLRLSCAASGFAYSTYSMGWFRQAPGKEREAVATINSGTFRLWYADSVKGRFTISRDNSKNTLYLQMNSLRAEDTAVYYCAARAWSPYSSTVDAGDFRYWGQGTLVTVSS

SEQ ID NO: 65 - protein, 126 aa, synthetic construct
  REGION 1..126: note = HuNb42 S75A
QVQLVESGGGLVQPGGSLRLSCAASGFAYSTYSMGWFRQAPGKEREAVATINSGTFRLWYTDSVKGRFTISRDNAKNTLYLQMNSLRAEDTAVYYCAARAWSPYSSTVDAGDFRYWGQGTLVTVSS

SEQ ID NO: 66 - protein, 126 aa, synthetic construct
  REGION 1..126: note = HuNb42 L79V
QVQLVESGGGLVQPGGSLRLSCAASGFAYSTYSMGWFRQAPGKEREAVATINSGTFRLWYTDSVKGRFTISRDNSKNTVYLQMNSLRAEDTAVYYCAARAWSPYSSTVDAGDFRYWGQGTLVTVSS

SEQ ID NO: 67 - protein, 126 aa, synthetic construct
  REGION 1..126: note = HuNb42 A88P
QVQLVESGGGLVQPGGSLRLSCAASGFAYSTYSMGWFRQAPGKEREAVATINSGTFRLWYTDSVKGRFTISRDNSKNTLYLQMNSLRPEDTAVYYCAARAWSPYSSTVDAGDFRYWGQGTLVTVSS

SEQ ID NO: 68 - protein, 126 aa, synthetic construct
  REGION 1..126: note = HuNb42 L121Q
QVQLVESGGGLVQPGGSLRLSCAASGFAYSTYSMGWFRQAPGKEREAVATINSGTFRLWYTDSVKGRFTISRDNSKNTLYLQMNSLRAEDTAVYYCAARAWSPYSSTVDAGDFRYWGQGTQVTVSS

SEQ ID NO: 69 - protein, 126 aa, synthetic construct
  REGION 1..126: note = HuNb42 T61A A88P
QVQLVESGGGLVQPGGSLRLSCAASGFAYSTYSMGWFRQAPGKEREAVATINSGTFRLWYADSVKGRFTISRDNSKNTLYLQMNSLRPEDTAVYYCAARAWSPYSSTVDAGDFRYWGQGTLVTVSS

SEQ ID NO: 70 - protein, 126 aa, synthetic construct
  REGION 1..126: note = HuNb42 A88P L115Q
QVQLVESGGGLVQPGGSLRLSCAASGFAYSTYSMGWFRQAPGKEREAVATINSGTFRLWYTDSVKGRFTISRDNSKNTLYLQMNSLRPEDTAVYYCAARAWSPYSSTVDAGDFRYWGQGTQVTVSS

SEQ ID NO: 71 - protein, 124 aa, synthetic construct
  REGION 1..124: note = aTNF-a mu
QVQLQDSGGGLVQAGGSLRLSCAASGGTFSSIIMAWFRQAPGKEREFVGAVSWSGGTTVYADSVLGRFEISRDSARKSVYLQMNSLKPEDTAVYYCAARPYQKYNWASASYNVWGQGTQVTVSS

SEQ ID NO: 72 - protein, 124 aa, synthetic construct
  REGION 1..124: note = aTNF-a mu 3MUT
QVQLQESGGGLVQAGGSLRLSCAASGGTFSSIIMAWFRQAPGKEREFVGAVSWSGGTTVYADSVKGRFTISRDSARKSVYLQMNSLKPEDTAVYYCAARPYQKYNWASASYNVWGQGTQVTVSS

SEQ ID NO: 73 - protein, 129 aa, synthetic construct
  REGION 1..129: note = aTNF-a VHH
QVQLQESGGGLVQPGGSLRLSCAASGRTFSDHSGYTYTIGWFRQAPGKEREFVARIYWSSGNTYYADSVKGRFAISRDIAKNTVDLTMNNLEPEDTAVYYCAARDGIPTSRSVESYNYWGQGTQVTVSS

SEQ ID NO: 74-80 - skipped (7 entries)

SEQ ID NO: 81 - protein, 119 aa, synthetic construct
  REGION 1..119: note = E1-1
EVQLQASGGGFVQPGGSLRLSCAASGGGSDAGTMGWFRQAPGKEREFVSAISWAGTAWRYYADSVKGRFTISRDNSKNTVYLQMNSLRAEDTATYYCALGSYEMDHHYWGQGTQVTVSS

SEQ ID NO: 82 - protein, 119 aa, synthetic construct
  REGION 1..119: note = E1-1 F11L
EVQLQASGGGLVQPGGSLRLSCAASGGGSDAGTMGWFRQAPGKEREFVSAISWAGTAWRYYADSVKGRFTISRDNSKNTVYLQMNSLRAEDTATYYCALGSYEMDHHYWGQGTQVTVSS

SEQ ID NO: 83 - protein, 119 aa, synthetic construct
  REGION 1..119: note = E1-1 S49A
EVQLQASGGGFVQPGGSLRLSCAASGGGSDAGTMGWFRQAPGKEREFVAAISWAGTAWRYYADSVKGRFTISRDNSKNTVYLQMNSLRAEDTATYYCALGSYEMDHHYWGQGTQVTVSS

SEQ ID NO: 84 - protein, 119 aa, synthetic construct
  REGION 1..119: note = E1-1 F11L S49A
EVQLQASGGGLVQPGGSLRLSCAASGGGSDAGTMGWFRQAPGKEREFVAAISWAGTAWRYYADSVKGRFTISRDNSKNTVYLQMNSLRAEDTATYYCALGSYEMDHHYWGQGTQVTVSS

SEQ ID NO: 85 - protein, 120 aa, synthetic construct
  REGION 1..120: note = E1-1 CDR
EVQLQASGGGFVQPGGSLRLSCAASGRRFSIEAMGWFRQAPGKEREFVSAIDSGGSTDYADSVKGRFTISRDNSKNTVYLQMNSLRAEDTATYYCAVIGSSWYGRGLDYWGQGTQVTVSS

SEQ ID NO: 86-90 - skipped (5 entries)

SEQ ID NO: 91 - protein, 125 aa, synthetic construct
  REGION 1..125: note = anti-VEGF VHH
DVQLVESGGGLVQPGGSLRLSCAASGRTFSSYSMGWFRQAPGKEREFVVAISKGGYKYDAVSLEGRFTISRDNAKNTVYLQINSLRPEDTAVYYCASSRAYGSSRLRLADTYEYWGQGTLVTVSS

SEQ ID NO: 92 - protein, 134 aa, synthetic construct
  REGION 1..134: note = anti-VEGF DARPIN
GSDLDKKLLEAARAGQDDEVRILMANGADVNARDSTGWTPLHLAAPWGHPEIVEVLLKNGADVNAADFQGWTPLHLAAAVGHLEIVEVLLKYGADVNAQDKFGKTAFDISIDNGNEDLAEILQKAAGGGSGGGS

SEQ ID NO: 93 - protein, 130 aa, synthetic construct
  REGION 1..130: note = anti-VEGF HuNb22 2MUT
QVQLVESGGGLVQPGGSLRLSCAASGYAYDTYYMGWFRQAPGKEREGVAGITSLVSGVAYYKYYTDSVKGRFTISRDNSKNTVDLQMNSLRAEDTAVYYCAASRSGLRARLLRPELYEYWGQGTLVTVSS

SEQ ID NO: 94 - protein, 127 aa, synthetic construct
  REGION 1..127: note = anti-VEGF HuNb23 3MUT
QVQLVESGGGLVQPGGSLRLSCVASGDTYSSACMGWFRQAPGKEREGVATICTSTSMRTRYYADSVKGRFTISRDNSKNTVYLQMNSLRAEDTAVYYCATGHTVGSSWRDPGAWRYWGQGTLVTVSS

SEQ ID NO: 95 - protein, 123 aa, synthetic construct
  REGION 1..123: note = anti-VEGF HuNb35 4MUT
QVQLVESGGGLVQPGGSLRLSCAASGLSYRPGYMGWFRQAPGKEREGVAIITTGGVTHYADSVKGRFTISRDNSKNTVYLQMNSLRAEDTAVYYCALANWVQFPLRVDGYKYWGQGTLVTVSS

SEQ ID NO: 96 - protein, 125 aa, synthetic construct
  REGION 1..125: note = Hu_aVEGF_VHH_3MUT
QVQLVESGGGLVQPGGSLRLSCAASGRTFSSYSMGWFRQAPGKEREFVAAISKGGYKYDAVSLEGRFTISRDNSKNTVYLQMNSLRPEDTAVYYCASSRAYGSSRLRLADTYEYWGQGTLVTVSS

SEQ ID NO: 97 - protein, 125 aa, synthetic construct
  REGION 1..125: note = Hu_aVEGF_VHH_5MUT
QVQLVESGGGLVQPGGSLRLSCAASGRTFSSYSMGWFRQAPGKEREFVAAISKGGYKYDAVSVKGRFTISRDNSKNTVYLQMNSLRPEDTAVYYCASSRAYGSSRLRLADTYEYWGQGTLVTVSS

SEQ ID NO: 98 - protein, 125 aa, synthetic construct
  REGION 1..125: note = Hu_aVEGF_VHH_6MUT
QVQLVESGGGLVQPGGSLRLSCAASGRTFSSYSMGWFRQAPGKEREFVAAISKGGYKYYAVSVKGRFTISRDNSKNTVYLQMNSLRPEDTAVYYCASSRAYGSSRLRLADTYEYWGQGTLVTVSS

SEQ ID NO: 99-100 - skipped (2 entries)

SEQ ID NO: 101 - protein, 150 aa, synthetic construct
  REGION 1..150: note = anti-TNFalpha VHH (human) - rigid
QVQLQESGGGLVQPGGSLRLSCAASGRTFSDHSGYTYTIGWFRQAPGKEREFVARIYWSSGNTYYADSVKGRFAISRDIAKNTVDLTMNNLEPEDTAVYYCAARDGIPTSRSVESYNYWGQGTQVTVSSPSTPPTPSPSTPPGGCDDDDK

SEQ ID NO: 102 - protein, 148 aa, synthetic construct
  REGION 1..148: note = anti-TNFalpha VHH (human)- aH
QVQLQESGGGLVQPGGSLRLSCAASGRTFSDHSGYTYTIGWFRQAPGKEREFVARIYWSSGNTYYADSVKGRFAISRDIAKNTVDLTMNNLEPEDTAVYYCAARDGIPTSRSVESYNYWGQGTQVTVSSAEAAAKEAAAKEAAAKAGC

SEQ ID NO: 103 - protein, 144 aa, synthetic construct
  REGION 1..144: note = anti-TNFalpha VHH (mouse)- aH
QVQLQDSGGGLVQAGGSLRLSCAASGGTFSSIIMAWFRQAPGKEREFVGAVSWSGGTTVYADSVLGRFEISRDSARKSVYLQMNSLKFPEDTAVYYCAARPYQKYNWASASYNVWGQGTQVTVSSAEAAAKEAAAKEAAAKAGC

SEQ ID NO: 104 - protein, 143 aa, synthetic construct
  REGION 1..143: note = anti-TNFalpha 3MUT VHH (mouse)- aH
QVQLQESGGGLVQAGGSLRLSCAASGGTFSSIIMAWFRQAPGKEREFVGAVSWSGGTTVYADSVKGRFTISRDSARKSVYLQMNSLKPEDTAVYYCAARPYQKYNWASASYNVWGQGTQVTVSSAEAAAKEAAAKEAAAKAGC

SEQ ID NO: 105 - protein, 62 aa, synthetic construct
  REGION 1..62: note = anti-TNFalpha affibody
CGGGVDNKFNKEVGWAFGEIGALPNLNALQFRAFIISLWDDPSQSANLLAEAKKLNDAQAPK

SEQ ID NO: 106 - protein, 265 aa, synthetic construct
  REGION 1..265: note = anti-IL-1beta scFv-rigid
EIVMTQSPSTLSASVGDRVIITCQASQSIDNWLSWYQQKPGKAPKLLIYRASTLASGVPSRFSGSGSGAEFTLTISSLQPDDFATYYCQNTGGGVSIAFGQGTKLTVLGGGGGSGGGGSGGGGSGGGGSEVQLVESGGGLVQPGGSLRLSCTASGFSLSSAAMAWVRQAPGKGLEWVGIIYDSASTYYASWAKGRFTISRDTSKNTVYLQMNSLRAEDTAVYYCARERAIFSGDFVLWGQGTLVTVSSSPSTPPTPSPSTPPGGC

SEQ ID NO: 107 - protein, 124 aa, synthetic construct
  REGION 1..124: note = Hu_aTNFaMu_3MUT
QVQLVESGGGLVQPGGSLRLSCAASGGTFSSIIMAWFRQAPGKEREFVGAVSWSGGTTVYADSVKGRFTISRDNSKNTVYLQMNSLRPEDTAVYYCAARPYQKYNWASASYNVWGQGTLVTVSS

SEQ ID NO: 108 - protein, 124 aa, synthetic construct
  REGION 1..124: note = Hu_aTNFaMu_5MUT
QVQLVESGGGLVQPGGSLRLSCAASGGTFSSIIMAWFRQAPGKEREFVAAISWSGGTTVYADSVKGRFTISRDNSKNTVYLQMNSLRPEDTAVYYCAARPYQKYNWASASYNVWGQGTLVTVSS

SEQ ID NO: 109 - protein, 129 aa, synthetic construct
  REGION 1..129: note = Hu_aTNFaHu_7MUT
QVQLVESGGGLVQPGGSLRLSCAASGRTFSDHSGYTYTMGWFRQAPGKEREFVARIYWSSGNTYYADSVKGRFTISRDNSKNTVYLQMNSLRPEDTAVYYCAARDGIPTSRSVESYNYWGQGTLVTVSS

SEQ ID NO: 110 - skipped

SEQ ID NO: 111 - protein, 124 aa, synthetic construct
  REGION 1..124: note = Hu_aEGFR_3MUT
EVQLVESGGGLVQPGGSLRLSCAASGRTSRSYGMGWFRQAPGKEREFVAGISWRGDSTGYADSVKGRFTISRDNSKNTVDLQMNSLRPEDTAVYYCAAAAGSAWYGTLYEYDYWGQGTLVTVSS

SEQ ID NO: 112 - protein, 120 aa, synthetic construct
  REGION 1..120: note = Hu_aHer2_3MUT
EVQLVESGGGLVQPGGSLRLSCAASGITFMRYAMGWYRQAPGKQREMVASINSGGTTNYADSVKGRFTISRDNSKNTVYLQMNSLRPEDTAVYYCNARWVKPQFIDNNYWGQGTLVTVSS

SEQ ID NO: 113 - protein, 119 aa, synthetic construct
  REGION 1..119: note = Hu_aPD1_102C3 3MUT
EVQLVESGGGLVQPGGSLRLSCAASGSIFSIHAMGWFRQAPGKEREFVAAITWSGGITYYEDSVKGRFTISRDNSKNTVYLQMNSLRPEDTAVYYCAADRAESSWYDYWGQGTLVTVSS

SEQ ID NO: 114 - protein, 119 aa, synthetic construct
  REGION 1..119: note = Hu_aPD1_102C12 3MUT
EVQLVESGGGLVQPGGSLRLSCAASGSIASIHAMGWFRQAPGKEREFVAVITWSGGITYYADSVKGRFTISRDNSKNTVYLQMNSLRPEDTAVYYCAGDKHQSSWYDYWGQGTLVTVSS

SEQ ID NO: 115 - protein, 119 aa, synthetic construct
  REGION 1..119: note = Hu_aPD1_102E2 3MUT
EVQLVESGGGLVQPGGSLRLSCAASGSISSIHAMGWFRQAPGKEREFVAAITWSGGITYYADSVKGRFTISRDNSKNTVYLQMNSLRPEDTAVYYCAADRAQSSWYDYWGQGTLVTVSS

SEQ ID NO: 116 - protein, 119 aa, synthetic construct
  REGION 1..119: note = Hu_aPD1_102E8 3MUT
EVQLVESGGGLVQPGGSLRLSCAASGSIFSINAMAWFRQAPGKEREFVALISWSGGSTYYEDSVKGRFTISRDNSKNTVYLQMNSLRPEDTAVYYCAADRVDSNWYDYWGQGTLVTVSS

SEQ ID NO: 117 - protein, 119 aa, synthetic construct
  REGION 1..119: note = Hu_aPD1_102H12 4MUT
EVQLVESGGGLVQPGGSLRLSCAASGRAFSSGTMGWFRQAPGKEREFVASIPWSGGRTYYADSVKGRFTISRDNSKNTVYLQMNSLRPEDTAVYYCAVKERSTGWDFAWGQGTLVTVSS

SEQ ID NO: 118 - protein, 119 aa, synthetic construct
  REGION 1..119: note = Hu aCaffeine 3MUT
EVQLVESGGGLVQPGGSLRLSCAASGRTGTIYSMAWFRQAPGKEREFLATIGWSSGITYYMDSVKGRFTISRDNSKNTVYLQMNSLRPEDTAVYYCAATRAYSVGYDYWGQGTLVTVSS

SEQ ID NO: 119 - protein, 124 aa, synthetic construct
  REGION 1..124: note = Hu_aEGFR_5MUT
QVQLVESGGGLVQPGGSLRLSCAASGRTSRSYGMGWFRQAPGKEREFVAGISWRGDSTGYADSVKGRFTISRDNSKNTVYLQMNSLRPEDTAVYYCAAAAGSAWYGTLYEYDYWGQGTLVTVSS

SEQ ID NO: 120 - protein, 114 aa, synthetic construct
  REGION 1..114: note = aAng2_D4
EVQLVESGGGLVQAGGSLRLSCVASGRIFTNTAMGWYRQAPGKWRELVATIYSGGSTKYIDSVKGRFIISRDNTRNTVHLQMNSLKPEDTAVYYCNTVGAGSYWGQGAQVTVSS

SEQ ID NO: 121 - protein, 114 aa, synthetic construct
  REGION 1..114: note = Hu_aAng2_D4_3MUT
QVQLVESGGGLVQPGGSLRLSCAASGRIFTNTAMGWYRQAPGKWRELVATIYSGGSTKYIDSVKGRFTISRDNSKNTVYLQMNSLRPEDTAVYYCATVGAGSYWGQGTLVTVSS

SEQ ID NO: 122 - protein, 126 aa, synthetic construct
  REGION 1..126: note = aAn2_H4
EVQLVESGGDLVQAGGSLRLSCAASGFTFGDFTIGWFRQAPGKEREGVSCINTGDGSTNYAESVKGRFTISSDNAKNTVYLQMNSLKPEDTAVYYCALDQAPMWSSWSAPYEYDYWGQGTQVTVSS

SEQ ID NO: 123 - protein, 126 aa, synthetic construct
  REGION 1..126: note = Hu_aAng2_H4_6MUT
QVQLVESGGGLVQPGGSLRLSCAASGFTFGDFTMGWFRQAPGKEREGVAAINTGDGSTNYADSVKGRFTISRDNSKNTVYLQMNSLRPEDTAVYYCALDQAPMWSSWSAPYEYDYWGQGTLVTVSS

SEQ ID NO: 124 - protein, 129 aa, synthetic construct
  REGION 1..129: note = aAng2_BI0027
EVQLVESGGGLVQAGGSLRLSCAASGFTLDDYAIGWFRQAPGKEREGVSSIRDNDGSTYYADSVKGRFTISSDNDKNTVYLQMNSLKPEDTAVYYCAAVPAGRLRFGEQWYPLYEYDAWGQGTLVTVSS

SEQ ID NO: 125 - protein, 129 aa, synthetic construct
  REGION 1..129: note = Hu_aAng2_0027_2MUT
QVQLVESGGGLVQPGGSLRLSCAASGFTLDDYAMGWFRQAPGKEREGVASIRDNDGSTYYADSVKGRFTISRDNSKNTVYLQMNSLRPEDTAVYYCAAVPAGRLRFGEQWYPLYEYDAWGQGTLVTVSS

SEQ ID NO: 126 - protein, 130 aa, synthetic construct
  REGION 1..130: note = aEpCam_Nb4
QVQLVQSGGGSVQGGASLRLSCAASGGERNNYCVAWFRQAPGKEREVAAISRAASGAQTTTKYYVDSVKGRFTISQDTKNTATVYLQMNSHKPEDTAYCTAKAKIYPPQCTGISRTIDYRGQGTQVTVSS

SEQ ID NO: 127 - protein, 129 aa, synthetic construct
  REGION 1..129: note = Hu_aEpCAM_Nb4_4MUT
QVQLVESGGGLVQPGGSLRLSCAASGGERNNYCMGWFRQAPGKEREVAAISRAASGAQTTTKYYVDSVKGRFTISRDNSKNTVYLQMNSLRPEDTAYCAAKAKIYPPQCTGISRTIDYRGQGTLVTVSS

SEQ ID NO: 128 - protein, 128 aa, synthetic construct
  REGION 1..128: note = aEpCam_Nb4
QVQLVQSGGGLVQGGASLRLSCAASSGRAGLIHVAWFRAKNTLYRQVAAISSDDTGFATLYYAVDSVKGRFTISQDTESTKYSYLQMNSLKPEDTIYCTAKLFSKTSYQKFIYKFNYWGQGTQVTVSS

SEQ ID NO: 129 - protein, 127 aa, synthetic construct
  REGION 1..127: note = Hu_aEpCAM_Nb5_11MUT
QVQLVESGGGLVQPGGSLRLSCAASGGRAGLIHMGWFRQAPGKYREVAAISSDDTGFATLYYAVDSVKGRFTISRDNSKNTVYLQMNSLRPEDTAYCTAKLFSKTSYQKFIYKFNYWGQGTLVTVSS

SEQ ID NO: 130 - protein, 128 aa, synthetic construct
  REGION 1..128: note = aEpCAM_Nb22
QVQQVQSGGGSVQAGASLRLSCAASSGLQQAIYVAWFRQAVGKEREVAAIGYWYWYHIQVYKYYVDSVKDRFTISGDTKSTKTYLQMNSLKNEDTAYYCAKIHHLPRQRQEHGIFDYRGQGTQVTVSS

SEQ ID NO: 131 - protein, 128 aa, synthetic construct
  REGION 1..128: note = Hu_aEpCAM_Nb22_6MUT
QVQLVESGGGLVQPGGSLRLSCAASGGLQQAIYMGWFRQAPGKEREVAAIGYWYWYHIQVYKYYVDSVKDRFTISRDNSKNTVYLQMNSLRPEDTAYYCAKIHHLPRQRQEHGIFDYRGQGTLVTVSS

SEQ ID NO: 132-140 - skipped (9 entries)

SEQ ID NO: 141 - protein, 121 aa, synthetic construct
  REGION 1..121: note = Hu2H10 5MUT CYS
QVQLVESGGGLVQPGGSLRLSCLASGRRFSIEAMGWYRQAPGKQRELVAIIDSGGSTDYVDSAKGRFTISRDNSKNTVYLQMNSLRAEDTAVYYCNVIGSSWYGRGLDYWGQGTLVTVSSC

SEQ ID NO: 142 - protein, 139 aa, synthetic construct
  REGION 1..139: note = Hu2H10 5MUT aH_CYS
QVQLVESGGGLVQPGGSLRLSCLASGRRFSIEAMGWYRQAPGKQRELVAIIDSGGSTDYVDSAKGRFTISRDNSKNTVYLQMNSLRAEDTAVYYCNVIGSSWYGRGLDYWGQGTLVTVSSAEAAAKEAAAKEAAAKAGC

SEQ ID NO: 143 - protein, 139 aa, synthetic construct
  REGION 1..139: note = Hu2H10 5MUT R86K A87P aH_CYS
QVQLVESGGGLVQPGGSLRLSCLASGRRFSIEAMGWYRQAPGKQRELVAIIDSGGSTDYVDSAKGRFTISRDNSKNTVYLQMNSLKPEDTAVYYCNVIGSSWYGRGLDYWGQGTLVTVSSAEAAAKEAAAKEAAAKAGC

SEQ ID NO: 144 - protein, 139 aa, synthetic construct
  REGION 1..139: note = Hu2H10 5MUT L115Q aH_CYS
QVQLVESGGGLVQPGGSLRLSCLASGRRFSIEAMGWYRQAPGKQRELVAIIDSGGSTDYVDSAKGRFTISRDNSKNTVYLQMNSLRAEDTAVYYCNVIGSSWYGRGLDYWGQGTQVTVSSAEAAAKEAAAKEAAAKAGC

SEQ ID NO: 145 - protein, 145 aa, synthetic construct
  REGION 1..145: note = HuNb42 A88P aH_CYS
QVQLVESGGGLVQPGGSLRLSCAASGFAYSTYSMGWFRQAPGKEREAVATINSGTFRLWYTDSVKGRFTISRDNSKNTLYLQMNSLRPEDTAVYYCAARAWSPYSSTVDAGDFRYWGQGTLVTVSSAEAAAKEAAAKEAAAKAGC

SEQ ID NO: 146 - protein, 145 aa, synthetic construct
  REGION 1..145: note = HuNb42 T61A A88P aH_CYS
QVQLVESGGGLVQPGGSLRLSCAASGFAYSTYSMGWFRQAPGKEREAVATINSGTFRLWYADSVKGRFTISRDNSKNTLYLQMNSLRPEDTAVYYCAARAWSPYSSTVDAGDFRYWGQGTLVTVSSAEAAAKEAAAKEAAAKAGC

SEQ ID NO: 147 - protein, 145 aa, synthetic construct
  REGION 1..145: note = HuNb42 A88P L115Q aH_CYS
QVQLVESGGGLVQPGGSLRLSCAASGFAYSTYSMGWFRQAPGKEREAVATINSGTFRLWYTDSVKGRFTISRDNSKNTLYLQMNSLRPEDTAVYYCAARAWSPYSSTVDAGDFRYWGQGTQVTVSSAEAAAKEAAAKEAAAKAGC

SEQ ID NO: 148 - protein, 65 aa, synthetic construct
  REGION 1..65: note = anti-TNFalpha affibody Gly
CGGGVDNKFNKEVGWAFGEIGALPNLNALQFRAFIISLWDDPSQSANLLAEAKKLNDAQAPKGGG

SEQ ID NO: 149 - protein, 143 aa, synthetic construct
  REGION 1..143: note = Hu_aTNFalphaMu_3MUT aH_CYS
QVQLVESGGGLVQPGGSLRLSCAASGGTFSSIIMAWFRQAPGKEREFVGAVSWSGGTTVYADSVKGRFTISRDNSKNTVYLQMNSLRPEDTAVYYCAARPYQKYNWASASYNVWGQGTLVTVSSAEAAAKEAAAKEAAAKAGC

SEQ ID NO: 150 - protein, 143 aa, synthetic construct
  REGION 1..143: note = Hu_aTNFalphaMu_5MUT aH_CYS
QVQLVESGGGLVQPGGSLRLSCAASGGTFSSIIMAWFRQAPGKEREFVAAISWSGGTTVYADSVKGRFTISRDNSKNTVYLQMNSLRPEDTAVYYCAARPYQKYNWASASYNVWGQGTLVTVSSAEAAAKEAAAKEAAAKAGC

SEQ ID NO: 151 - protein, 148 aa, synthetic construct
  REGION 1..148: note = Hu_aTNFalphaHu_7MUT aH_CYS
QVQLVESGGGLVQPGGSLRLSCAASGRTFSDHSGYTYTMGWFRQAPGKEREFVARIYWSSGNTYYADSVKGRFTISRDNSKNTVYLQMNSLRPEDTAVYYCAARDGIPTSRSVESYNYWGQGTLVTVSSAEAAAKEAAAKEAAAKAGC

SEQ ID NO: 152 - protein, 152 aa, synthetic construct
  REGION 1..152: note = IL-2_C125S aH_CYS
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLTAEAAAKEAAAKEAAAKAGC

SEQ ID NO: 153 - protein, 133 aa, synthetic construct
  REGION 1..133: note = IL-15_5MUT aH_CYS
NWVNVISDLKKIEDLIQSMHIDATLYTESDVHPSCKVTAMQCFLSELQVISLESGDASIHDTVENLTILANNSLSSNGYVTESGCKECEELEAKNIKEFLQSFVHIVQMFINTSAEAAAKEAAAKEAAAKAGC

SEQ ID NO: 154 - protein, 166 aa, synthetic construct
  REGION 1..166: note = aTNFa_DARPin G3S_CYS
DLGKKLLEVARAGQDDEVRILMANGADVNAADHQSFTPLHLYAIFGHLEIVEVLLKNGADVNASDWHGNTPLHLAAWIGHLEIVEVLLKYGADVNATDHSGSTPLHLAATLGHLEIVEVLLKYGADVNAQDKFGKTAFDISIDNGNEDLAEILQKAAGGGSGGGSC

SEQ ID NO: 155 - protein, 116 aa, synthetic construct
  REGION 1..116: note = aPDL1_Nb1
QVQLQESGGGLVQAGGSLRLSCATSGSIFSIISMGWYRQAPGKQRELVALVFRGGSTVYADSVKGRFTISGDIAKNTVYLQMDSLKPEDTAVYYCNVKSIGTAQYWGQGTQVTVSS

SEQ ID NO: 156 - protein, 116 aa, synthetic construct
  REGION 1..116: note = Hu_aPDL1_Nb1_4MUT
QVQLVESGGGLVQPGGSLRLSCAASGSIFSIISMGWYRQAPGKQRELVALIFRGGSTVYADSVKGRFTISRDNSKNTVYLQMNSLRPEDTAVYYCAVKSIGTAQYWGQGTLVTVSS

SEQ ID NO: 157 - protein, 113 aa, synthetic construct
  REGION 1..113: note = aPDL1_Nb104E10
EVQLVESGGGLVQAGGSLRLSCAASGVDASNSAMGWYRQAPGKQREWVARITGGGLIAYTDSVKGRFTISRDNAKSTVYLQMNSLEPEDTAVYYCNTINSRDWGQGTQVTVSS

SEQ ID NO: 158 - protein, 113 aa, synthetic construct
  REGION 1..113: note = Hu_aPDL1_Nb104E10_1MUT
QVQLVESGGGLVQPGGSLRLSCAASGVDASNSAMGWYRQAPGKQREWVARITGGGLIAYTDSVKGRFTISRDNSKSTVYLQMNSLRPEDTAVYYCATINSRDWGQGTLVTVSS

SEQ ID NO: 159 - protein, 119 aa, synthetic construct
  REGION 1..119: note = aPDL1_NbG5
QVQLQESGGGLVQPGGSLRLSCAASGSFFRTYIVSWYRQAPGKRREAVAVMSNSGNTNYADSVKGRFTISRDNAVNTVYLQMNSLKPEDTAVYYCNLLKVTVVPPANYWGQGTQVTVSS

SEQ ID NO: 160 - protein, 119 aa, synthetic construct
  REGION 1..119: note = Hu_aPDL1_NbG5_4MUT
QVQLVESGGGLVQPGGSLRLSCAASGSFFRTYIMGWYRQAPGKRREAVAVISNSGNTNYADSVKGRFTISRDNSKNTVYLQMNSLRPEDTAVYYCALLKVTVVPPANYWGQGTLVTVSS

SEQ ID NO: 161 - protein, 119 aa, synthetic construct
  REGION 1..119: note = aPDL2_103E4
EVQLVESGGGLVQAGGSLRLSCAASGSTFSNYVSNYAMGWGRQAPGTQRELVASISNGDTTNYADSVKGRFTISRDNAKNTVYLQMNSLKPEDTAVYYCFEHQVAGLTWGQGTQVTVSS

SEQ ID NO: 162 - protein, 119 aa, synthetic construct
  REGION 1..119: note = Hu_aPDL2_103E4_2MUT
QVQLVESGGGLVQPGGSLRLSCAASGSTFSNYVSNYAMGWGRQAPGKQRELVASISNGDTTNYADSVKGRFTISRDNSKNTVYLQMNSLRPEDTAVYYCAEHQVAGLTWGQGTLVTVSS

SEQ ID NO: 163 - protein, 127 aa, synthetic construct
  REGION 1..127: note = aPDL2_103E5
EVQLVESGGGLVQTGGSLRLSCAASGFTLDYYGIGWFRQAPGKEREGVSFISGSDGSTYYAESVKGRFTISRDKAKNTVYLQMNSLKPEDTAVYYCAADPWGPPSIATMTSYEYKHWGQGTQVTVSS

SEQ ID NO: 164 - protein, 127 aa, synthetic construct
  REGION 1..127: note = Hu_aPDL2_103E5_4MUT
QVQLVESGGGLVQPGGSLRLSCAASGFTLDYYGMGWFRQAPGKEREGVAFISGSDGSTYYAESVKGRFTISRDNSKNTVYLQMNSLRPEDTAVYYCAADPWGPPSIATMTSYEYKHWGQGTLVTVSS

SEQ ID NO: 165 - protein, 120 aa, synthetic construct
  REGION 1..120: note = aPDL2_Nb103F10
EVQLVESGGGLVQAGGSLRLSCAASGRTFSSGTMGWFRRAPGKEREFVASIPWSGGRTYYADSVKDRFTISRDNAQNTVFLQMNSLKPEDTAVYYCAFKERSTGWDFASWGQGIQVTVSS

SEQ ID NO: 166 - protein, 120 aa, synthetic construct
  REGION 1..120: note = Hu_aPDL2_Nb103F10_5MUT
QVQLVESGGGLVQPGGSLRLSCAASGRTFSSGTMGWFRQAPGKEREFVASIPWSGGRTYYADSVKGRFTISRDNSKNTVYLQMNSLRPEDTAVYYCAFKERSTGWDFASWGQGTLVTVSS

SEQ ID NO: 167 - protein, 114 aa, synthetic construct
  REGION 1..114: note = Hu_aIL-1B_5C_5MUT
QVQLVESGGGLVQPGGSLRLSCAASGGYYYSSFYWGWFRQAPGQGLEAVATIPYYYTYYTDSVKGRFTISRDNSKNTVYLQMNSLRPEDTAVYYCASYNIHNFWGQGTLVTVSS

SEQ ID NO: 168 - protein, 113 aa, synthetic construct
  REGION 1..113: note = Hu_aIL-1B_6C_3MUT
QVQLVESGGGLVQPGGSLRLSCAASGSYSYSYSWGWFRQAPGQGLEAVAAIVYYYTYYADSVKGRFTISRDNSKNTVYLQMNSLRPEDTAVYYCASYAPFPFWGQGTLVTVSS

SEQ ID NO: 169 - protein, 112 aa, synthetic construct
  REGION 1..112: note = Hu_aIL-1B_11H_3MUT
QVQLVESGGGLVQPGGSLRLSCAASGGYSYYSWGWFRQAPGQGLEAVASIAYYYTYYADSVKGRFTISRDNSKNTVYLQMNSLRPEDTAVYYCAYSDIISYWGQGTLVTVSS

SEQ ID NO: 170 - protein, 112 aa, synthetic construct
  REGION 1..112: note = Hu_aIL-1B_12D_2MUT
QVQLVESGGGLVQPGGSLRLSCAASGSYYSSSYGGWFRQAPGQGLEAVATIPYYTSYADSVKGRFTISRDNSKNTVYLQMNSLRPEDTAVYYCASFFPFFYWGQGTLVTVSS

(19) United States
(12) Patent Application Publication
(10) Pub. No.: US 2023/0405133 A1
(43) Pub. Date: Dec. 21, 2023

(54) PURIFIED MULTIVALENT PROTEIN-HYALURONIC ACID POLYMER CONJUGATES

(71) Applicant: Valitor, Inc., Berkeley, CA (US)

(72) Inventors: Wesley M. JACKSON, Berkeley, CA (US); Amy A. TWITE, Berkeley, CA (US); Adam BARNEBEY, Berkeley, CA (US); Livia Wilz BRIER, Berkeley, CA (US); Jesse M. MCFARLAND, Berkeley, CA (US)

(21) Appl. No.: 18/300,831

(22) Filed: Apr. 14, 2023

Related U.S. Application Data
(60) Provisional application No. 63/331,548, filed on Apr. 15, 2022.

(51) Int. Cl. A61K 47/61; C08G 65/48; A61K 47/22

(52) U.S. Cl. CPC A61K 47/61; C08G 65/48; A61K 47/22

(57) ABSTRACT

The present invention relates to purified hyaluronic acid conjugates comprising biologically active peptides. The invention further relates to pharmaceutical compositions of the conjugates, and methods of preparing them.

### CROSS-REFERENCES TO RELATED APPLICATIONS

This application claims priority to U.S. Provisional Application No. 63/331,548, filed Apr. 15, 2022, which is incorporated herein in its entirety for all purposes.

### SEQUENCE LISTING

The material in the accompanying sequence listing is hereby incorporated by reference in its entirety. The accompanying file, named 2023-04-11 Sequence Listing_ST26 052566-507001US.xml was created on Apr. 11, 2023, and is 187,801 bytes in size.

### BACKGROUND OF THE INVENTION

The use of biopolymers to modify the properties of biologically active agents is a recurring theme across a wide range of medical and biological applications. A variety of chemical linkers can be used to attach bioactive peptides or proteins to biopolymers to modify the pharmacological properties of the resulting conjugate for use as a drug that can provide optimal treatment of specific diseases. Peptide-polymer conjugate comprising multiple copies of one or more species of peptide conjugated to a single biopolymer chain have been employed to impart specific improvements to the pharmacological properties of the peptides, including: (1) higher binding affinity to the biological target, (2) slower diffusivity through a target tissue, and (3) inhibition of proteases that could deactivate the biological activity of the peptides or proteins.

These improved pharmacological properties of peptide-polymer conjugates are particularly useful for the delivery of potent drugs that are be delivered directly into the diseased tissue. The dose delivered directly into the tissue can be lower than would be required to achieve the same therapeutic effect after systemic administration because the drug has been administered locally to the target tissue. It is also possible to administer to drugs to tissues that otherwise have poor transport properties from the blood. Specific examples of tissues where direct drug administration is common include the posterior eye chamber via intravitreal injection and articular joints via intra-articular injection.

However, local tissue administration requires a professional to safely provide the required injection, which makes them more burdensome and costly to administer compared to systemic administration. When the peptide drug is administered as part of a peptide-polymer conjugate, it is possible to substantially reduce the frequency of drug administration, thereby reducing the burden on the patient to receive effective treatment. Furthermore, a reduction in the number of local injections reduces the risk of local tissue injury or adverse effects to the injection. Finally, the need for less frequent administrations can reduce the amount of time that the drug concentration in the target tissue is below the therapeutic concentration, thereby improving the overall efficacy of the drug. Based on these advantages, there is a strong motivation to develop protein-polymer drug products for a variety of diseases.

The synthesis of multivalent Sonic hedgehog proteins conjugated to hyaluronic acid through a N-ε-maleimidocaproic acid hydrazide linker has been reported. See, Wall, S. T. et al. Bioconjugate Chemistry 2008, 19, 806-812.

To appropriately formulate a peptide-polymer conjugate as a drug product, it is necessary to achieve sufficiently high drug concentrations to enable appropriate dosing in the patient. It is also necessary to prepare purified peptide-polymer conjugates that exhibit high bioactivity and shelf-stability, for example, by being able to remain in solution for up to two years from the date of manufacture to the date of clinical use. Interactions between the peptide-polymer conjugates can negatively impact the ability to complete any of these drug-enabling properties.

The methods used to attach the polymer and the peptides can have a substantial impact on the pharmacological properties of the conjugates, intra-conjugate interactions, as well as conjugate-to-conjugate interactions. Therefore, there is a need to develop purified peptide-polymer conjugates with specific linker methods that will enable them to achieve the preferred pharmacological properties for a given disease as well as to be successfully formulated into a drug product. The present invention meets this and other needs.

### BRIEF SUMMARY OF THE INVENTION

In some embodiments, the conjugate of the present invention is a conjugate that is a random polymer of Formula III:

(X—Y—Z¹)ₙ—(Z²)ₚ—(Z³)_{q} (III)
having a molecular weight of from about 0.1 MDa to about 3 MDa;
wherein
each X is independently a peptide having a molecular weight of from about 5 kDa to about 200 kDa;
each Y is an organic linker;
each X—Y—Z¹ moiety has the structure:

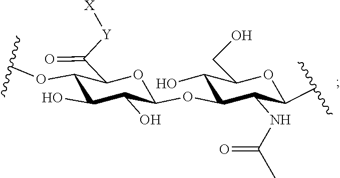

each Z² has the structure:

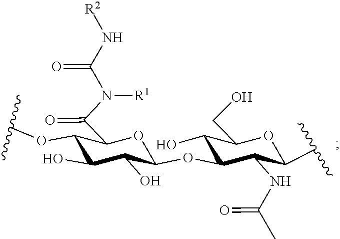

each Z³ independently has the structure:

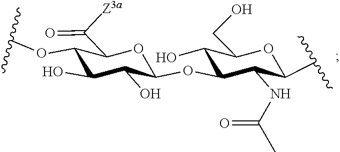

each R¹ and R² is independently C₁-C₆ alkyl, —(C₁-C₆ alkyl)-NR³R⁴, or C₅-C₈ cycloalkyl;
each R³ and R⁴ is independently H or C₁-C₆ alkyl;
each Z^{3a} is independently OH or Y′;
each Y′ is an unreacted organic linker;
subscript n is an integer of from 1 to 1500 and less than about 15% of the sum of subscripts n, p, and q;
subscript p is an integer of from 0 to 1000 and less than about 10% of the sum of subscripts n, p, and q; and
subscript q is an integer of from 100 to 10000.

In some embodiments, the conjugate is a conjugate that is a random polymer of Formula IIIa:

(X¹—X²—Y—Z¹)ₙ—(Z²)ₚ—(Z³)_{q} (IIIa),
having a molecular weight of about 0.8 MDa;
wherein
each X¹ is a peptide having an anti-VEGF amino acid sequence comprising SEQ ID NO: 67;
each X² is a peptide linker having an amino acid sequence comprising

(SEQ ID NO: 21)
AEAAAKEAAAKEAAAKAGC;

each Y is an organic linker having the structure:

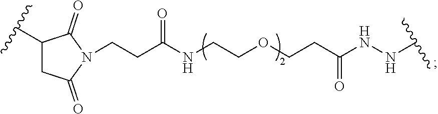

each X¹—X²—Y—Z¹ moiety has the structure:

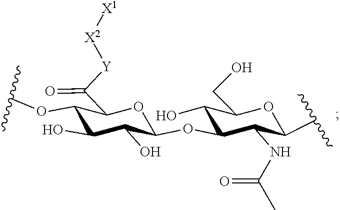

each Z² has the structure:

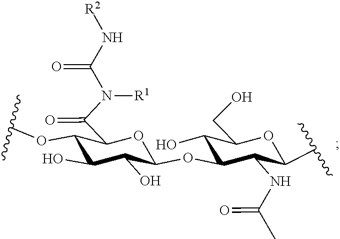

each Z³ independently has the structure:

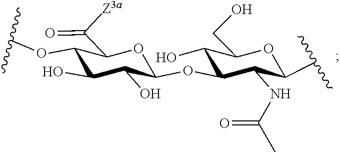

each Z^{3a} is independently OH or Y′;
each Y′ has the structure:

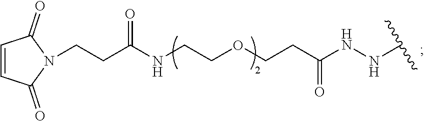

each R¹ and R² is ethyl or —(CH₂)₃—NMe₂;
subscript n is an integer of from 10 to 300 and less than about 10% of the sum of subscripts n,
p, and q; subscript p is an integer of from 1 to 15 and less than about 0.5% of the sum of subscripts n,
p, and q; and subscript q is an integer of from 1000 to 3000.

In some embodiments, the pharmaceutical composition comprises a conjugate as described herein, or a pharmaceutically acceptable salt thereof, and a pharmaceutically acceptable excipient.

In some embodiments, the method of the present invention is a method of treating an ocular disorder in a subject in need thereof, comprising administering to the subject a conjugate as described herein.

In some embodiments, the method of the present invention is a method of treating a disease or disorder in an articular joint in a subject in need thereof, comprising administering to the subject a conjugate as described herein.

In some embodiments, a method of preparing a conjugate of the present invention comprises: (a) forming a first reaction mixture comprising a hyaluronic acid polymer having a molecular weight of from about 0.1 MDa to about 3 MDa, from about 0.1 to about 2 equivalents coupling agent per hyaluronic acid monomer, and an organic linker agent of formula H₂N—R^{Y}, wherein R^{Y} is

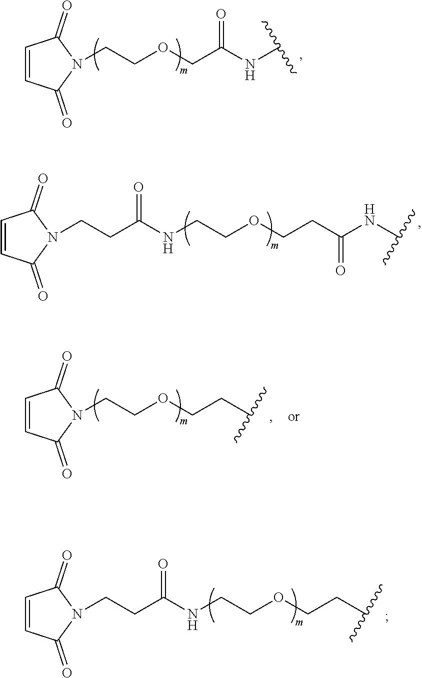

subscript m is an integer of from 1 to 300; thereby forming an intermediate polymer having a plurality of monomers of Formula IV:

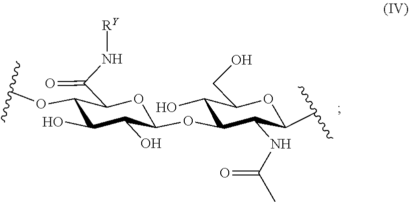

(b) forming a second reaction mixture comprising the intermediate polymer and a peptide having a molecular weight of from about 5 kDa to about 200 kDa, wherein the peptide comprises one or more —SH; thereby preparing the conjugate.

### BRIEF DESCRIPTION OF THE DRAWINGS

FIG. 1A shows an expression open reading frame (ORF). FIG. 1B shows soluble expression (mAU*mL) of Hu2H10 or 2H10 containing different peptide linkers. FIG. 1C shows protein expression by SDS-PAGE of Hu2H10_5MUT (SEQ ID NO: 55), Hu2H10_5MUT_CYS (SEQ ID NO: 141), or Hu2H10_5MUT_aH_CYS (SEQ ID NO: 142). FIG. 1D shows soluble expression (fold process yield) of HuNb42_A88P (SEQ ID NO: 67) or HuNb42_A88P aH_CYS (SEQ ID NO: 145).

FIG. 2 shows the amino acid sequence of 2H10 and point mutation variants.

FIG. 3 shows the protein expression of Hu2H10 mutants. Y-axis shows immobilized-metal affinity chromatography (IMAC) peak area (mAU*mL). X-axis depicts protein expression of Hu2H10_5MUT (“WT (5MUT)”) (SEQ ID NO: 55), Hu2H10_5MUT_R86K_A87P (“R86K_A87P”) (SEQ ID NO: 56), Hu2H10_5MUT_L115Q (“L115Q”) (SEQ ID NO: 57), Hu2H10_5MUT_R86K_A87P_L115Q (“R86K_A87P_L115Q”) (SEQ ID NO: 58).

FIG. 4 shows the amino acid sequence of Nb42 and point mutation variants.

FIG. 5 shows Coomassie brilliant blue (CBB) staining of E. coli cell extracts that expressed HuNb42 and point mutation variants.

FIG. 6 shows relative E. coli cell culture yield of HuNb42 (SEQ ID NO: 61) and HuNb42 A88P (SEQ ID NO: 67) point mutation variant in cytoplasm and periplasm.

FIG. 7 shows relative humanness of Nb42 (SEQ ID NO: 61), HuNb42 (SEQ ID NO: 62), and HuNb42 A88P (SEQ ID NO: 67), as compared to caplacizumab, bevacizumab, and ranibizumab.

FIG. 8 shows Coomassie brilliant blue (CBB) staining of E. coli cell extracts that expressed aTNFaMu (SEQ ID NO: 71) or aTNFaMu_3MUT (SEQ ID NO: 72) at RT, 50° C., 60° C., 70° C., and 80° C.

FIG. 9 shows the relative protein expression of E1-1 (SEQ ID NO: 81) and point mutants E1-1 F11L (SEQ ID NO: 82), E1-1 S49A (SEQ ID NO: 83), E1-1 F11L/S49A (SEQ ID NO: 84), and E1-1 CDR (SEQ ID NO: 85).

FIG. 10A shows Coomassie brilliant blue (CBB) staining of purified HuNb42 protein (SEQ ID NO: 62) induced at 18° C. and 37° C. FIG. 10B shows purified Hu2H10_5MUT protein (SEQ ID NO: 55) induced at 16° C., 27° C., 30° C., and 37° C.

FIG. 11A shows two preparations of HuNb42 A88P (SEQ ID NO: 67), without (“220119”) and with (“220204”) EDTA treatment. FIG. 11B shows recovery of TNFα 3MUT (SEQ ID NO: 104) before (“Input”) and after treatment with EDTA and filtration through 50 kDa (“50 kDa FT”) or 100 kDa (“100 kDa FT”) polyethersulfone membranes (left), and endotoxin levels before (“Input”) and after filtration through 50 kDa (“50 kDa FT”) or 100 kDa (“100 kDa FT”) polyethersulfone membranes (right). FIG. 11C shows SDS-PAGE gel of purified aAng2_D4_aH_CYS (SEQ ID NO: 120).

FIG. 12 shows Rh distributions of DARPin multivalent proteins (MVPs) prepared by Method 1 (Conjugate 5, Conjugate 2) and Method 5 (Conjugate 3, Conjugate 6, Conjugate 7). The MVPs prepared using Method 5 exhibited smaller MVP radius.

FIG. 13A-13B show activity of the MVPs. FIG. 13A shows biolayer interferometry (BLI) VEGF association and dissociation curves for anti-VEGF MVPs made using different intermediates and the anti-VEGF E1-1 peptide. Top: Method 2 Conjugate 10 (K_{D}=0.123 nM) association, and dissociation curves. Bottom: Method 5 Conjugate 8 (K_{D}=0.184 nM) showing baseline, association, and dissociation curves for different concentrations of Conjugate 8 (x-axis: time in seconds, y axis: BLI signal). Both conjugates show similar binding kinetic curves and calculated dissociation constant. FIG. 13B shows VEGF binding affinity (“KD”, nM) of two peptides as compared to the corresponding MVPs. First bar: unconjugated 2H10_5MUT_aH_CYS (SEQ ID NO: 142); second bar: unconjugated HuNb42_A88P_aH_CYS (SEQ ID NO: 145); third bar: MVP containing 2H10_5MUT_aH_CYS (SEQ ID NO: 142); fourth bar: MVP containing HuNb42_A88P_aH_CYS (SEQ ID NO: 145).

FIG. 14 shows SEC traces for MVP stability samples at various ages (x-axis: retention time, y-axis: absorbance at 280 nm). Examples of changes in SEC retention time for anti-VEGF DARPin MVPs made with intermediates synthesized using Method 5 (Conjugate 1, top) or Method 1 (Conjugate 2, bottom), stored for up to 71 days at 37° C. MVP synthesized with Method 5 (top) showed larger losses in radius with aging.

FIG. 15 shows changes in radius of gyration for DARPin MVPs made using intermediate Method 1 (Conjugate 2, upper line) or Method 5 (Conjugate 3, lower line) after aging under accelerated conditions for 28 and 32 days at 37° C.

FIG. 16 shows VEGF binding constant (K_{D}) for BI VHH anti-VEGF MVPs made using intermediate Method 1, Method 2, or Method 5 before and after aging at 37° C. By day 4 of the stability study, the MVPs synthesized with Method 5 intermediates no longer bound VEGF. BLI limit of detection (LOD) is 0.001 nM. Samples with this value listed in the figure were below the BLI LOD for K_{D}.

FIG. 17 shows changes in association constant (Kₒₙ) for DARPin MVPs made using intermediate Method 1 (Conjugate 2, square, upper line) or Method 5 (Conjugate 1, triangle, lower line) after aging under accelerated conditions for 28 and 32 days at 37° C. The lower Kₒₙ and slower association initially and with aging is observed in Conjugate 1, suggesting the low impurity polymer of Conjugate 2 was stabilizing and enhancing the Kₒₙ.

FIG. 18 shows in vivo half-life extension VHH and MVPs synthesized with Method 1 intermediate after intravitreal injection in rabbits. Conjugate 12 anti-TNFα VHH MVP (Method 1 intermediate) rabbit intravitreal pharmacokinetics (IVT PK) study, >2X half-life extension vs unconjugated. n=3 eyes per timepoint. All eyes received 50 μg of VHH.

### DETAILED DESCRIPTION OF THE INVENTION

### I. General

The present invention provides purified peptide-hyaluronic acid polymer conjugates using linkers to covalently link each peptide to the polymer, and methods of preparing the same. The purified peptide-hyaluronic acid conjugates exhibit higher stability compared to previously described conjugates.

### II. Definitions

Unless specifically indicated otherwise, all technical and scientific terms used herein have the same meaning as commonly understood by those of ordinary skill in the art to which this invention belongs. In addition, any method or material similar or equivalent to a method or material described herein can be used in the practice of the present invention. For purposes of the present invention, the following terms are defined.

“About” when referring to a value includes the stated value+/−10% of the stated value. For example, about 50% includes a range of from 45% to 55%, while about 20 molar equivalents includes a range of from 18 to 22 molar equivalents. Accordingly, when referring to a range, “about” refers to each of the stated values+/−10% of the stated value of each end of the range. For instance, a ratio of from about 1 to about 3 (weight/weight) includes a range of from 0.9 to 3.3.

“Alkyl” is a linear or branched saturated monovalent or divalent hydrocarbon. For example, an alkyl group can have 1 to 10 carbon atoms (i.e., C₁₋₁₀ alkyl) or 1 to 8 carbon atoms (i.e., C_{1_8} alkyl) or 1 to 6 carbon atoms (i.e., C₁₋₆ alkyl) or 1 to 4 carbon atoms (i.e., (C₁₋₄ alkyl). Examples of alkyl groups include, but are not limited to, methyl (Me, —CH₃), ethyl (Et, —CH₂CH₃), 1-propyl (n-Pr, n-propyl, —CH₂CH₂CH₃), 2-propyl (i-Pr, i-propyl, —CH(CH₃)₂), 1-butyl (n-Bu, n-butyl, —CH₂CH₂CH₂CH₃), 2-methyl-1-propyl (i-Bu, i-butyl, —CH₂CH(CH₃)₂), 2-butyl (s-Bu, s-butyl, —CH(CH₃)CH₂CH₃), 2-methyl-2-propyl (t-Bu, t-butyl, —C(CH₃)₃), 1-pentyl (n-pentyl, —CH₂CH₂CH₂CH₂CH₃), 2-pentyl (—CH(CH₃)CH₂CH₂CH₃), 3-pentyl (—CH(CH₂CH₃)₂), 2-methyl-2-butyl (—C(CH₃)₂CH₂CH₃), 3-methyl-2-butyl (—CH(CH₃)CH(CH₃)₂), 3-methyl-1-butyl (—CH₂CH₂CH(CH₃)₂), 2-methyl-1-butyl (—CH₂CH(CH₃)CH₂CH₃), 1-hexyl (—CH₂CH₂CH₂CH₂CH₂CH₃), 2-hexyl (—CH(CH₃)CH₂CH₂CH₂CH₃), 3-hexyl (—CH(CH₂CH₃)(CH₂CH₂CH₃)), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂), 3,3-dimethyl-2-butyl (—CH(CH₃)C(CH₃)₃, and octyl (—(CH₂)₇CH₃).

“Cycloalkyl” refers to a single saturated or partially unsaturated all carbon ring having 3 to 20 annular carbon atoms (i.e., C₃₋₂₀ cycloalkyl), for example from 3 to 12 annular atoms, for example from 3 to 10 annular atoms, or 3 to 8 annular atoms, or 3 to 6 annular atoms, or 3 to 5 annular atoms, or 3 to 4 annular atoms. The term “cycloalkyl” also includes multiple condensed, saturated and partially unsaturated all carbon ring systems (e.g., ring systems comprising 2, 3 or 4 carbocyclic rings). Accordingly, cycloalkyl includes multicyclic carbocycles such as a bicyclic carbocycles (e.g., bicyclic carbocycles having about 6 to 12 annular carbon atoms such as bicyclo[3.1.0]hexane and bicyclo[2.1.1]hexane), and polycyclic carbocycles (e.g. tricyclic and tetracyclic carbocycles with up to about 20 annular carbon atoms). The rings of a multiple condensed ring system can be connected to each other via fused, spiro and bridged bonds when allowed by valency requirements. Non-limiting examples of monocyclic cycloalkyl include cyclopropyl, cyclobutyl, cyclopentyl, 1-cyclopent-1-enyl, 1-cyclopent-2-enyl, 1-cyclopent-3-enyl, cyclohexyl, 1-cyclohex-1-enyl, 1-cyclohex-2-enyl and 1-cyclohex-3-enyl.

“Organic linker” as used herein refers to a chemical moiety that directly or indirectly covalently links the peptide to the polymer. Organic linkers useful in the present invention can be about 100 Da to 500 Da. The types of organic linkers of the present invention include, but are not limited to, imides, amides, amines, esters, carbamates, ureas, thioethers, thiocarbamates, thiocarbonate and thioureas. One of skill in the art will appreciate that other types of organic linkers are useful in the present invention.

“Thiol” refers to the —SH functional group.

“Thiol reactive group” refers to a group capable of reacting with a thiol to form a covalent bond to the sulfur atom. Representative thiol reactive groups include, but are not limited to, thiol, TNB-thiol, haloacetyl, aziridine, acryloyl, vinylsulfone, APN (3-arylpropiolonitrile), maleimide and pyridyl disulfide. Reaction of the thiol reactive group with a thiol can form a disulfide or a thioether.

“Coupling agent” as used herein refers to a reagent that effects reaction between a carboxylic acid (—(C═O)—OH) and an amine (—NH₂) group to form an amide (—(C═O)—NH—).

“Peptide,” “polypeptide,” and “protein” are used interchangeably herein, and refer to naturally occurring and synthetic amino acids of any length, as well as amino acid analogs and amino acid mimetics that function in a manner similar to the naturally occurring amino acids. The term “peptide” includes fusion proteins, including, but not limited to, fusion proteins with a heterologous amino acid sequence, fusions with heterologous and homologous leader sequences, with or without N-terminal methionine residues; immunologically tagged proteins; and the like. Peptides further include post-translationally modified peptides.

“VHH” as used herein refers to a single-domain heavy chain antibody.

“DARPin” refers to a designed ankyrin repeat protein, which is a genetically engineered antibody mimetic protein that can exhibit highly specific and high-affinity target protein binding.

An “alpha-helix” or “α-helix” is a common motif in the secondary structure of proteins and is a right hand-helix conformation in which every backbone N—H group hydrogen bonds to the backbone C═O group of the amino acid located four residues earlier along the protein sequence. The alpha-helix is also known as a classic Pauling-Corey-Branson α-helix, or 3.6₁₃-helix, which denotes the average number of residues per helical turn (3.6) with 13 atoms being involved in the ring formed by the hydrogen bond. Peptides that contain an alpha-helix is said to be alpha-helical. Such peptides may be partly or entirely alpha-helical. As understood in the art, an alpha-helix has at least four amino acid residues. In some embodiments, an alpha-helix has from 4 to 40 amino acids.

Provided are also pharmaceutically acceptable salts of the peptides or conjugates described herein. “Pharmaceutically acceptable” or “physiologically acceptable” refer to compounds, salts, compositions, dosage forms and other materials which are useful in preparing a pharmaceutical composition that is suitable for veterinary or human pharmaceutical use.

“Pharmaceutical composition” as used herein refers to a product comprising the specified ingredients in the specified amounts, as well as any product, which results, directly or indirectly, from combination of the specified ingredients in the specified amounts. The pharmaceutical composition is generally safe for biological use.

“Pharmaceutically acceptable excipient” as used herein refers to a substance that aids the administration of an active agent to an absorption by a subject. Pharmaceutically acceptable excipients useful in the present invention include, but are not limited to, binders, fillers, disintegrants, lubricants, coatings, sweeteners, flavors and colors. One of skill in the art will recognize that other pharmaceutically acceptable excipients are useful in the present invention.

The conjugates described herein may be prepared and/or formulated as pharmaceutically acceptable salts or when appropriate as a free base. Pharmaceutically acceptable salts are non-toxic salts of a free base form of a compound that possess the desired pharmacological activity of the free base. These salts may be derived from inorganic or organic acids or bases. For example, a conjugate that contains a basic nitrogen may be prepared as a pharmaceutically acceptable salt by contacting the compound with an inorganic or organic acid. Non-limiting examples of pharmaceutically acceptable salts include sulfates, pyrosulfates, bisulfates, sulfites, bisulfites, phosphates, monohydrogen-phosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, methylsulfonates, propylsulfonates, besylates, xylenesulfonates, naphthalene-1-sulfonates, naphthalene-2-sulfonates, phenylacetates, phenylpropionates, phenylbutyrates, citrates, lactates, γ-hydroxybutyrates, glycolates, tartrates, and mandelates. Lists of other suitable pharmaceutically acceptable salts are found in Remington: The Science and Practice of Pharmacy, 21St Edition, Lippincott Wiliams and Wilkins, Philadelphia, Pa., 2006.

Examples of “pharmaceutically acceptable salts” of the conjugates disclosed herein also include salts derived from an appropriate base, such as an alkali metal (for example, sodium, potassium), an alkaline earth metal (for example, magnesium), ammonium and NR₄⁺ (wherein R is C₁-C₄ alkyl). Also included are base addition salts, such as sodium or potassium salts.

“Therapeutically effective amount” as used herein refers to a dose that produces therapeutic effects for which it is administered. The exact dose will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins). In sensitized cells, the therapeutically effective dose can be lower than the conventional therapeutically effective dose for non-sensitized cells.

“Inhibition”, “inhibits” and “inhibitor” as used herein refer to a compound that prohibits or a method of prohibiting, a specific action or function.

“Treatment” or “treat” or “treating” as used herein refers to an approach for obtaining beneficial or desired results. For purposes of the present disclosure, beneficial or desired results include, but are not limited to, alleviation of a symptom and/or diminishment of the extent of a symptom and/or preventing a worsening of a symptom associated with a disease or condition. In one embodiment, “treatment” or “treating” includes one or more of the following: a) inhibiting the disease or condition (e.g., decreasing one or more symptoms resulting from the disease or condition, and/or diminishing the extent of the disease or condition); b) slowing or arresting the development of one or more symptoms associated with the disease or condition (e.g., stabilizing the disease or condition, delaying the worsening or progression of the disease or condition); and c) relieving the disease or condition, e.g., causing the regression of clinical symptoms, ameliorating the disease state, delaying the progression of the disease, increasing the quality of life, and/or prolonging survival.

“Prophylaxis” refers to preventing or retarding the progression of clinical illness in patients suffering from a disease.

A “subject” of the present invention is a mammal, which can be a human or a non-human mammal, for example a companion animal, such as a dog, cat, rat, or the like, or a farm animal, such as a horse, donkey, mule, goat, sheep, pig, or cow, and the like. In some embodiments, the subject is human.

“Articular joint” as used herein refers to the fibrous or cartilaginous joints, which is a fibrous or cartilaginous area wherein two or more bones connect to each other.

“Diffusion half-life” as used herein refers to the time it takes for the initial concentration of the conjugate within a given volume or space to decrease by half, where the decrease in concentration is a function of the concentration gradient.

“Intra-articular half-life” as used herein refers to the time it takes for the initial concentration of the conjugate within a particular joint to decrease by half, where the transport out of the joint is via convection. Convective transport is the combination of transport via diffusion and advection, where advective transport is the transport of a substance by bulk motion.

### III. Peptides

In some embodiments, the peptides of the present invention offer advantages to comparative peptides in the art, for example, higher degree of humanness, greater solubility, greater stability, lower tendency to aggregate in solution, and/or higher expression levels in convenient systems such as E. coli.

In some embodiments, the peptide is a peptide having Formula (I):

FR1-CDR1-FR2-CDR2-FR3-CDR3-FR4 (I),
CDR1, CDR2, and CDR3 are each independently complementarity-determining regions;
FR1 has an amino acid sequence comprising

(SEQ ID NO: 1)
X¹⁰VQLX¹¹EX¹²GGGX¹³X¹⁴QX¹⁵GX¹⁶SLRLSCX¹⁷X¹⁸SG,

wherein
X¹⁰ is Q, E, or D,
X¹¹ is V, Q, A, or E,
X¹² is S or T,
X¹³ is L, S, or V,
X¹⁴ is V or A,
X¹⁵ is P, A, or T,
X¹⁶ is G, D, or R,
X¹⁷ is A, V, T, or E, and
X¹⁸ is A or V;
FR2 has an amino acid sequence comprising X²⁰X²¹WX²²RQX²³PGKX²⁴X²⁵EX²⁶VX²⁷X²⁸I (SEQ ID NO: 2),
   wherein
   X²⁰ is M, I, V, or L,
   X²¹ is G, S, or A,
   X²² is F, Y, or V,
   X²³ is A, V, P, or T,
   X²⁴ is E, G, A, or Q,
   X²⁵ is R or L,
   X²⁶ is F, G, W, or L,
   X²⁷ is A, G, or S, and
   X²⁸ is A, S, or G;
FR3 has an amino acid sequence comprising

(SEQ ID NO: 3)
YX³⁰DSVKGRFTISX³¹DX³²X³³KX³⁴X³⁵VX³⁶LQMX³⁷X³⁸LRX³⁹ªEDTA
X^{39b}YYCAA,

wherein
X³⁰ is A, G, S, or T,
X³¹ is R or Q,
X³² is N, S, or D,
X³³ is S, A, or D,
X³⁴ is N or K,
X³⁵ is T or M,
X³⁶ is Y, D, or S,
X³⁷ is N or D,
X³⁸ is S or N,
X³⁹a is P or A, and
X^{39b} is V, M, L, or I; and
FR4 has an amino acid sequence comprising YWGX⁴⁰GTX⁴¹VTVSS (SEQ ID NO: 4),
   wherein
   X⁴⁰ is Q or K, and
   X⁴¹ is L or Q.

In some embodiments, X¹³ is L.

In some embodiments, X²⁷ is A.

In some embodiments, X³⁰ is A.

In some embodiments, X³⁹a is P.

In some embodiments, X⁴⁰ is Q.

In some embodiments, FR1 has an amino acid sequence comprising

(SEQ ID NO: 5)
QVQLVESGGGLVQPGGSLRLSCAASG.

In some embodiments, FR2 has an amino acid sequence comprising

(SEQ ID NO: 6)
MGWFRQAPGKEREFVAAI.

In some embodiments, FR3 has an amino acid sequence comprising

(SEQ ID NO: 7)
YADSVKGRFTISRDNSKNTVYLQMNSLRPEDTAVYYCAA;

In some embodiments, FR4 has an amino acid sequence comprising

(SEQ ID NO: 8)
YWGQGTLVTVSS.

In some embodiments, FR1 has an amino acid sequence comprising

(SEQ ID NO: 5)
QVQLVESGGGLVQPGGSLRLSCAASG;

FR2 has an amino acid sequence comprising MGWFRQAPGKEREFVAAI (SEQ ID NO: 6);
FR3 has an amino acid sequence comprising

(SEQ ID NO: 7)
YADSVKGRFTISRDNSKNTVYLQMNSLRPEDTAVYYCAA;

FR4 has an amino acid sequence comprising YWGQGTLVTVSS (SEQ ID NO: 8).

In some embodiments, CDR1, CDR2, and CDR3 are each complementarity-determining regions from an antibody or a cytokine. In some embodiments, the antibody is a monoclonal IgG, an IgG fragment, single chain scFv, single-domain heavy-chain VHH, adnectin, affibody, anticalin, DARPin, or an engineered Kunitz-type inhibitor. In some embodiments, the complementarity-determining regions are each specific to vascular endothelial growth factor (VEGF), tumor necrosis factor-alpha (TNF-α), programmed cell death protein 1 (PD-1), programmed death ligand-1 (PD-L1), cytotoxic T-lymphocyte-associated protein 4 (CTLA4), cluster of differentiation 40 (CD40), cluster of differentiation 134 (CD134), cluster of differentiation 137 (CD137), glucocorticoid-induced tumor necrosis factor receptor-related protein (GITR), V-domain immunoglobulin suppressor of T-cell activation (VISTA), T cell immunoglobulin and mucin domain-containing protein 3 (TIM-3), lymphocyte activating 3 (LAG3), interleukin-1-beta (IL-1P), interleukin-6 (IL-6), interleukin-10 (IL-10), interleukin-12 (IL-12), or interleukin-15 (IL-15). In some embodiments, the complementarity-determining regions are each specific to vascular endothelial growth factor (VEGF).

In some embodiments, the peptide consists of Formula I.

In some embodiments, the peptide has one or more of the following: (a) a CDR1 of 7 amino acids in length; (b) a CDR2 of 7 or 8 amino acids in length; and/or (c) a CDR3 of 9 to 16 amino acids in length.

In some embodiments of the peptide, (a) CDR1 has an amino acid sequence comprising FAYSTYS (SEQ ID NO: 9), CDR2 has an amino acid sequence comprising NSGTFRLW (SEQ ID NO: 10), and CDR3 has an amino acid sequence comprising RAWSPYSSTVDAGDFR (SEQ ID NO: 11); or
(b) CDR1 has an amino acid sequence comprising RRFSIEA (SEQ ID NO: 12), CDR2 has an amino acid sequence comprising DSGGSTD (SEQ ID NO: 13), and CDR3 has an amino acid sequence comprising IGGSWYGRGLD (SEQ ID NO: 14); or
(c) CDR1 has an amino acid sequence comprising GTFSSII (SEQ ID NO: 15), CDR2 has an amino acid sequence comprising SWSGGTTV (SEQ ID NO: 16), and CDR3 has an amino acid sequence comprising RPYQKYNWASASYNV (SEQ ID NO: 17); or
(d) CDR1 has an amino acid sequence comprising GGSDAGT (SEQ ID NO: 18), CDR2 has an amino acid sequence comprising SWAGTAWR (SEQ ID NO: 19), and CDR3 has an amino acid sequence comprising LGSYEMDHH (SEQ ID NO: 20).

In some embodiments, the amino acid sequence comprises any one of SEQ ID NOS: 51-58, 61-73, 81-85, 91-98, 101-109, 111-131, and 141-170. In some embodiments, the peptide has an amino acid sequence comprising SEQ ID NO: 55. In some embodiments, the peptide has an amino acid sequence comprising SEQ ID NO: 67. In some embodiments, the peptide has an amino acid sequence comprising SEQ ID NO: 142. In some embodiments, the peptide has an amino acid sequence comprising SEQ ID NO: 145.

In some embodiments, the peptide has an amino acid sequence comprising any one of SEQ ID NOS: 51-58, 61-73, 81-85, 91-95, 101-106, and 111-118. In some embodiments, the peptide has an amino acid sequence comprising any one of SEQ ID NOS: 73, 81, 91, and 92.

In some embodiments, the peptide has an amino acid sequence comprising any one of SEQ ID NOS: 101-106. In some embodiments, the peptide has an amino acid sequence comprising SEQ ID NO: 67.

### IV. Conjugates

In some embodiments, the conjugate is a conjugate of Formula IIa:

(X¹—X²—Y)ₙ—Z (IIa),
wherein
   each X¹ is independently a peptide as described herein;
   each X² is independently a peptide linker of from 3 to 100 amino acids in length;
   each Y is independently an organic linker;
   Z is a biocompatible polymer having a molecular weight of from about 0.1 MDa to about 3 MDa; and
   subscript n is an integer of from 1 to 1500.

In some embodiments, the conjugate is a conjugate of Formula IIb:

(X¹—X^{2A}—Y)ₙ—Z (IIb),
wherein
   each X¹ is independently a peptide having a molecular weight of from about 5 kDa to about 200 kDa;
   each X^{2A} is independently a peptide linker that comprises an alpha-helix;
   each Y is independently an organic linker;
   Z is a biocompatible polymer having a molecular weight of from about 0.1 MDa to about 3 MDa; and
   subscript n is an integer of from 1 to 1500.

In some embodiments, each X¹ is independently a peptide of the present invention.

In some embodiments, each peptide linker is independently from 7 to 100 amino acids in length. In some embodiments, each peptide linker is independently from 10 to 30 amino acids in length.

In some embodiments, each peptide linker independently has an amino acid sequence comprising:

(SEQ ID NO: 21)
AEAAAKEAAAKEAAAKAGC,
(SEQ ID NO: 22)
AEEEKRKAEEEKRKAEEEAGC,
(SEQ ID NO: 23)
AEEEKRKAEEEKRKAEEEKRKAEEEAGC,
(SEQ ID NO: 24)
AEEEEKKKKEEEEKKKKAGC,
(SEQ ID NO: 25)
AEAAAKEAAAKAGC,
(SEQ ID NO: 26)
PSRLEEELRRRLTEGC,
or
(SEQ ID NO: 27)
AEEEEKKKQQEEEAERLRRIQEEMEKERKRREEDEERRRKEEEERRMKL
EMEAKRKQEEEERKKREDDEKRKKKAGC.

In some embodiments, each peptide linker has an amino acid sequence comprising

(SEQ ID NO: 21)
AEAAAKEAAAKEAAAKAGC;

Each peptide can be linked to the biocompatible polymer by a variety of organic linkers generally known in the art for forming antibody-drug conjugates, such as those provided by Conju-Probe or BroadPharm of San Diego, CA, or Creative Biolabs of Shirley, NY. Methods for forming bioconjugate bonds are described in Bioconjugate Techniques, 3^{rd} Edition, Greg T. Hermanson. The organic linkers can be reactive with amines, carbonyls, carboxyl and activated esters, can react via Click-chemistry (with or without copper), or be reactive with thiols.

Representative organic linkers include an amide or disulfide, or are formed from a reactive group such as succinic anhydride, succinimide, N-hydroxy succinimide, N-chlorosuccinimide, N-bromosuccinimide, maleic anhydride, maleimide, hydantoin, phthalimide, and others. The organic linkers useful in the present invention are small and generally have a molecular weight from about 100 Da to about 500 Da containing two functional groups consisting of a maleimide and either an amine or hydrazide. In some embodiments, the peptide is covalently linked to the polymer via a sulfide bond and an organic linker having a molecular weight of from about 100 Da to about 500 Da. In some embodiments, the organic linker has a molecular weight of from about 100 Da to about 300 Da. In some embodiments, the organic linker comprises a succinimide. In some embodiments, the organic linker is formed using N-beta-maleimidopropionic acid hydrazide (BMPH), N-epsilon-maleimidocaproic acid hydrazide (EMCH), N-aminoethylmaleimide, N-kappa-maleimidoundecanoic acid hydrazide (KUMH), hydrazide-PEG2-maleimide, amine-PEG2-maleimide, hydrazide-PEG3-maleimide, or amine-PEG3-maleimide.

Representative organic linkers include, but are not limited to,

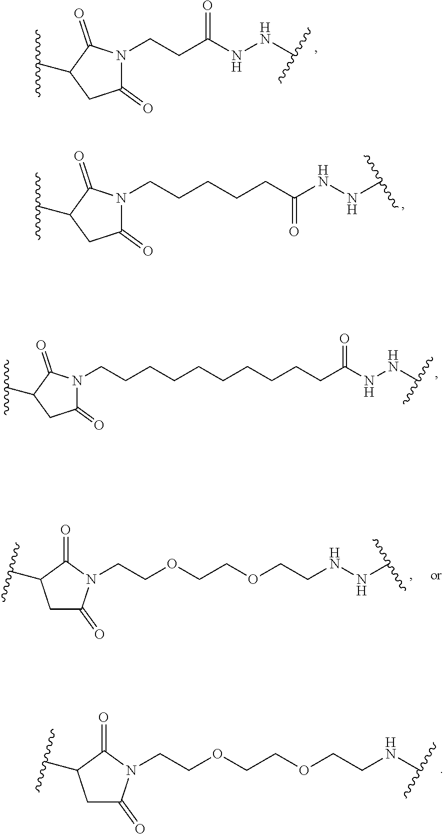 [SMILES: *NNC(=O)CCN1C(=O)CC(*)C1=O ; *NNC(=O)CCCCCN1C(=O)CC(*)C1=O ; *NNC(=O)CCCCCCCCCCN1C(=O)CC(*)C1=O ; *NNCCOCCOCCN1C(=O)CC(*)C1=O ; *NCCOCCOCCN1C(=O)CC(*)C1=O]

In some embodiments, the organic linker can be N-epsilon-maleimidocaproic acid hydrazide (EMCH):

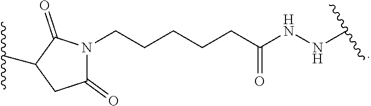 [SMILES: *NNC(=O)CCCCCN1C(=O)CC(*)C1=O]

In some embodiments, the organic linker has the structure:

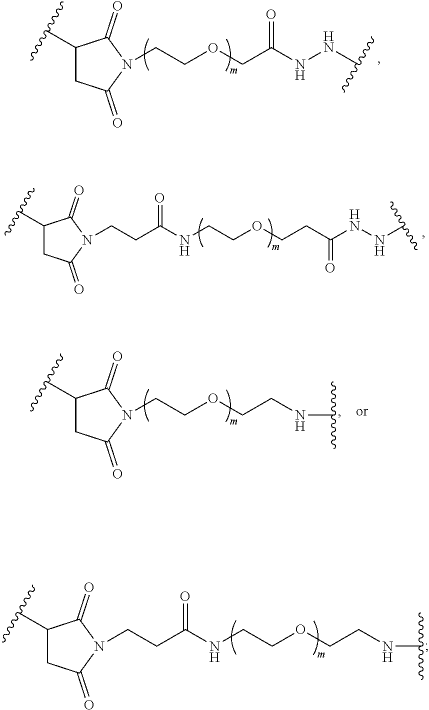

wherein subscript m is an integer of from 1 to 300. In some embodiments, subscript m is an integer from 1 to 100.

In some embodiments, the organic linker has the structure:

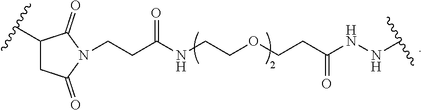

In some embodiments, preparing the conjugates of the present invention comprises covalently attaching the organic linker to the biocompatible polymer and then covalently attaching the peptide to the organic linker. In some embodiments, after preparing the conjugate of the present invention, unreacted organic linker is present on the biocompatible polymer. The structure of the unreacted organic linker depends on the organic linker and would be understood by a person skilled in the art.

Representative unreacted organic linkers include, but are not limited to,

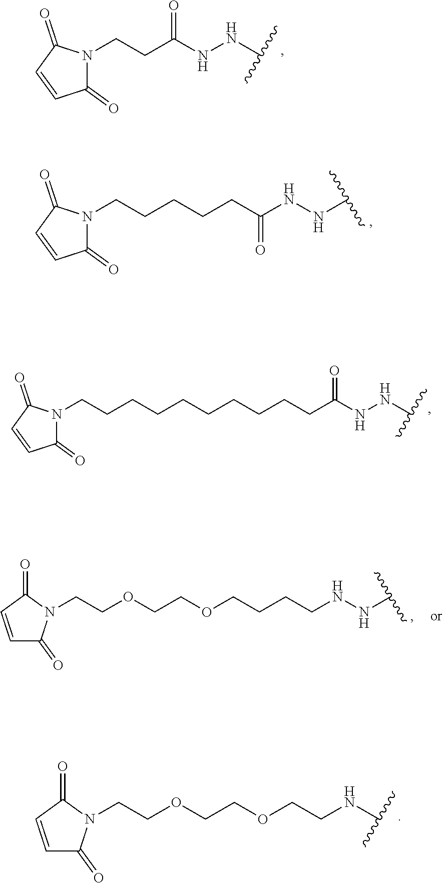 [SMILES: *NNC(=O)CCN1C(=O)C=CC1=O ; *NNC(=O)CCCCCN1C(=O)C=CC1=O ; *NNC(=O)CCCCCCCCCCN1C(=O)C=CC1=O ; *NNCCCCOCCOCCN1C(=O)C=CC1=O ; *NCCOCCOCCN1C(=O)C=CC1=O]

In some embodiments, the unreacted organic linker has the structure:

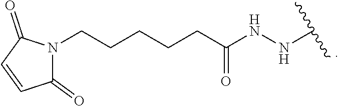 [SMILES: *NNC(=O)CCCCCN1C(=O)C=CC1=O]

In some embodiments, the unreacted organic linker has the structure:

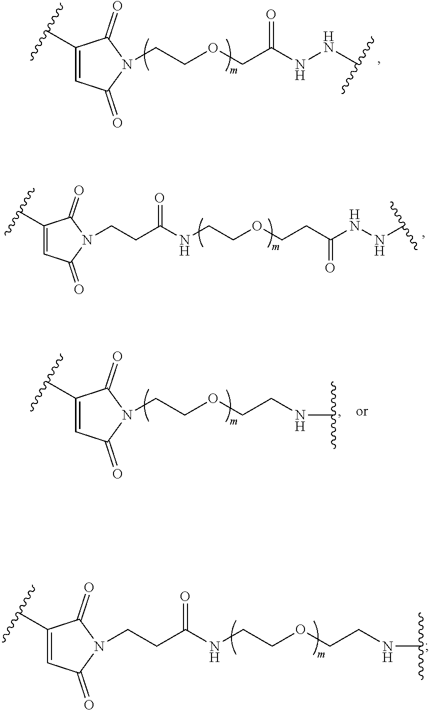

wherein subscript m is an integer of from 1 to 300. In some embodiments, subscript m is an integer from 1 to 100.

In some embodiments, the unreacted organic linker has the structure:

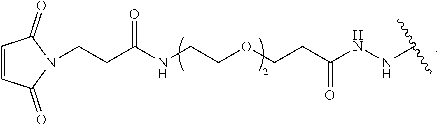

In some embodiments, the biocompatible polymer is a polysaccharide.

In some embodiments, the biocompatible polymer is a glycosaminoglycan.

In some embodiments, the biocompatible polymer is hyaluronic acid.

In some embodiments, the biocompatible polymer has a molecular weight of from about 0.4 MDa to about 2 MDa. In some embodiments, the biocompatible polymer has a molecular weight of from about 0.7 MDa to about 1.5 MDa. In some embodiments, the biocompatible polymer has a molecular weight of about 0.8 MDa.

In some embodiments, subscript n is an integer of from 1 to 1500. In some embodiments, subscript n is an integer of from 5 to 1000. In some embodiments, subscript n is an integer of from 10 to 400. In some embodiments, subscript n is an integer of from 10 to 100.

In some embodiments, the conjugate is a conjugate of Formula IIa:

(X¹—X²—Y)ₙ—Z (IIa),
wherein
each X¹ is independently a peptide as described herein;
each X² is a peptide linker having an amino acid sequence comprising

(SEQ ID NO: 21)
AEAAAKEAAAKEAAAKAGC;

each Y is an organic linker having the structure:

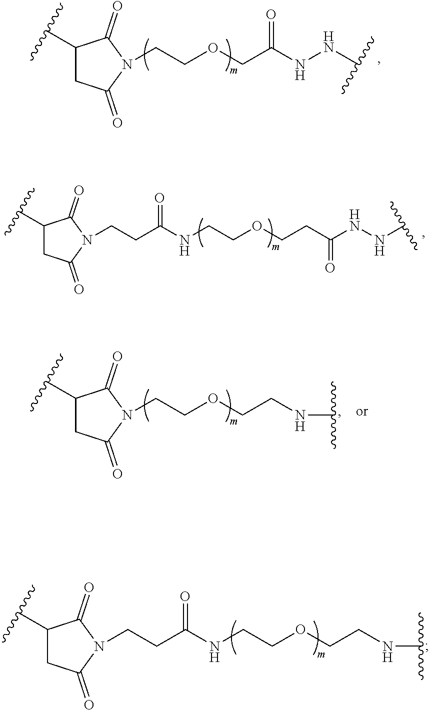

Z is a biocompatible polymer that is a hyaluronic acid having a molecular weight of from about 0.1 MDa to about 3 MDa;
subscript m is an integer of from 1 to 300; and
subscript n is an integer of from 1 to 1500.

In some embodiments, the conjugate of the present invention is a conjugate that is a random polymer of Formula III:

(X—Y—Z¹)ₙ—(Z²)ₚ—(Z³)_{q} (III),
having a molecular weight of from about 0.1 MDa to about 3 MDa; wherein
each X is independently a peptide having a molecular weight of from about 5 kDa to about 200 kDa;
each Y is an organic linker;
each X—Y-Z¹ moiety has the structure:

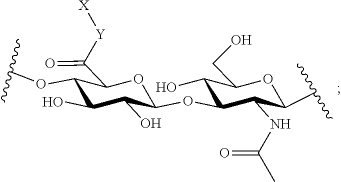

each Z² has the structure:

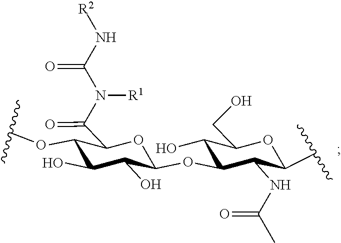

each Z³ independently has the structure:

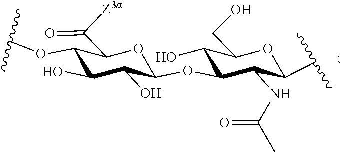

each R¹ and R² is independently C₁-C₆ alkyl, —(C₁-C₆ alkyl)-NR³R⁴, or C₅-C₅ cycloalkyl;
each R³ and R⁴ is independently H or C₁-C₆ alkyl;
each Z^{3a} is independently OH or Y′;
each Y′ is an unreacted organic linker;
subscript n is an integer of from 1 to 1500 and less than about 15% of the sum of subscripts n,
p, and q; subscript p is an integer of from 0 to 1000 and less than about 10% of the sum of subscripts n,
p, and q; and subscript q is an integer of from 100 to 10000.

In some embodiments, each X is a peptide having an amino acid sequence comprising any one of SEQ ID NOS: 51-58, 61-73, 81-85, 91-98, 101-109, 111-131, and 141-170. In some embodiments, each X is a peptide having an amino acid sequence comprising SEQ ID NO: 55. In some embodiments, each X is a peptide having an amino acid sequence comprising SEQ ID NO: 67. In some embodiments, each X is a peptide having an amino acid sequence comprising SEQ ID NO: 142. In some embodiments, each X is a peptide having an amino acid sequence comprising SEQ ID NO: 145.

In some embodiments, each X is a peptide having an amino acid sequence comprising any one of SEQ ID NOS: 51-58, 61-73, 81-85, 91-95, 101-106, and 111-118.

In some embodiments, the conjugate has the structure of Formula IIIa:

(X¹—X²—Y—Z¹)ₙ(Z²)ₚ—(Z³)_{q} (IIIa),
wherein
each X¹ is independently a peptide having a molecular weight of from about 5 kDa to about 200 kDa; and
each X² is a peptide linker that comprises an alpha-helix.

In some embodiments, each X¹ comprises a peptide having Formula I:

FR1-CDR1-FR2-CDR2-FR3-CDR3-FR4 (I),
CDR1, CDR2, and CDR3 are each independently complementarity-determining regions;
FR1 has an amino acid sequence comprising

(SEQ ID NO: 1)
X¹⁰VQLX¹¹EX¹²GGGX¹³X¹⁴QX¹⁵GX¹⁶SLRLSCX¹⁷X¹⁸SG,

wherein
X¹⁰ is Q, E, or D,
X¹¹ is V, Q, A, or E,
X¹² is S or T,
X¹³ is L, S, or V,
X¹⁴ is V or A,
X¹⁵ is P, A, or T,
X¹⁶ is G, D, or R,
X¹⁷ is A, V, T, or E, and
X¹⁸ is A or V;
FR2 has an amino acid sequence comprising X²⁰X²¹WX²²RQX²³PGKX²⁴X²⁵EX²⁶VX²⁷X²⁸I (SEQ ID NO: 2),
   wherein
   X²⁰ is M, I, V, or L,
   X²¹ is G, S, or A,
   X²² is F, Y, or V,
   X²³ is A, V, P, or T,
   X²⁴ is E, G, A, or Q,
   X²⁵ is R or L,
   X²⁶ is F, G, W, or L,
   X²⁷ is A, G, or S, and
   X²⁸ is A, S, or G;
FR3 has an amino acid sequence comprising

(SEQ ID NO: 3)
YX³⁰DSVKGRFTISX³¹
DX³²X³³KX³⁴X³⁵VX³⁶LQMX³⁷X³⁸LRX^{39a}EDTAX^{39b}YYCAA,

wherein
X³⁰ is A, G, S, or T,
X³¹ is R or Q,
X³² is N, S, or D,
X³³ is S, A, or D,
X³⁴ is N or K,
X³⁵ is T or M,
X³⁶ is Y, D, or S,
X³⁷ is N or D,
X³⁸ is S or N,
X^{39a} is P or A, and
X^{39b} is V, M, L, or I; and
FR4 has an amino acid sequence comprising YWGX⁴⁰GTX⁴¹VTVSS (SEQ ID NO: 4),
   wherein
   X⁴⁰ is Q or K, and
   X⁴¹ is L or Q.

In some embodiments, X¹³ is L.

In some embodiments, X²⁷ is A.

In some embodiments, X³⁰ is A.

In some embodiments, X^{39a} is P.

In some embodiments, X⁴⁰ is Q.

In some embodiments,
FR1 has an amino acid sequence comprising QVQLVESGGGLVQPGGSLRLSCAASG (SEQ ID NO: 5).

In some embodiments, FR2 has an amino acid sequence comprising

(SEQ ID NO: 6)
MGWFRQAPGKEREFVAAI.

In some embodiments, FR3 has an amino acid sequence comprising

(SEQ ID NO: 7)
YADSVKGRFTISRDNSKNTVYLQMNSLRPEDTAVYYCAA.

In some embodiments, FR4 has an amino acid sequence comprising YWGQGTLVTVSS (SEQ ID NO: 8).

In some embodiments, FR1 has an amino acid sequence comprising

(SEQ ID NO: 5)
QVQLVESGGGLVQPGGSLRLSCAASG;

FR2 has an amino acid sequence comprising MGWFRQAPGKEREFVAAI (SEQ ID NO: 6);
FR3 has an amino acid sequence comprising

(SEQ ID NO: 7)
YADSVKGRFTISRDNSKNTVYLQMNSLRPEDTAVYYCAA;

and
FR4 has an amino acid sequence comprising YWGQGTLVTVSS (SEQ ID NO: 8).

In some embodiments, CDR1, CDR2, and CDR3 are each complementarity-determining regions from an antibody or a cytokine.

In some embodiments, the antibody is a monoclonal IgG, an IgG fragment, single chain scFv, single-domain heavy-chain VHH, adnectin, affibody, anticalin, DARPin, or an engineered Kunitz-type inhibitor. In some embodiments, the antibody is a monoclonal IgG.

In some embodiments, the antibody is an IgG fragment. In some embodiments, the antibody is a single-domain heavy-chain VHH. In some embodiments, the antibody is a DARPin.

In some embodiments, the complementarity-determining regions are each specific to vascular endothelial growth factor (VEGF), tumor necrosis factor-alpha (TNF-α), programmed cell death protein 1 (PD-1), programmed death ligand-1 (PD-L1), cytotoxic T-lymphocyte-associated protein 4 (CTLA4), cluster of differentiation 40 (CD40), cluster of differentiation 134 (CD134), cluster of differentiation 137 (CD137), glucocorticoid-induced tumor necrosis factor receptor-related protein (GITR), V-domain immunoglobulin suppressor of T-cell activation (VISTA), T cell immunoglobulin and mucin domain-containing protein 3 (TIM-3), lymphocyte activating 3 (LAG3), interleukin-i-beta (IL-1R), interleukin-6 (IL-6), interleukin-10 (IL-10), interleukin-12 (IL-12), or interleukin-15 (IL-15). In some embodiments, the complementarity-determining regions are each specific to vascular endothelial growth factor (VEGF). In some embodiments, the complementarity-determining regions are each specific to tumor necrosis factor-alpha (TNF-α). In some embodiments, the complementarity-determining regions are each specific to interleukin-1-beta (IL-1).

In some embodiments, the peptide consists of Formula I.

In some embodiments, the peptide has one or more of the following: (a) a CDR1 of 7 amino acids in length; (b) a CDR2 of 7 or 8 amino acids in length; and/or (c) a CDR3 of 9 to 16 amino acids in length.

In some embodiments of the peptide,
(a) CDR1 has an amino acid sequence comprising FAYSTYS (SEQ ID NO: 9), CDR2 has an amino acid sequence comprising NSGTFRLW (SEQ ID NO: 10), and CDR3 has an amino acid sequence comprising RAWSPYSSTVDAGDFR (SEQ ID NO: 11); or
(b) CDR1 has an amino acid sequence comprising RRFSIEA (SEQ ID NO: 12), CDR2 has an amino acid sequence comprising DSGGSTD (SEQ ID NO: 13), and CDR3 has an amino acid sequence comprising IGGSWYGRGLD (SEQ ID NO: 14); or
(c) CDR1 has an amino acid sequence comprising GTFSSII (SEQ ID NO: 15), CDR2 has an amino acid sequence comprising SWSGGTTV (SEQ ID NO: 16), and CDR3 has an amino acid sequence comprising RPYQKYNWASASYNV (SEQ ID NO: 17); or
(d) CDR1 has an amino acid sequence comprising GGSDAGT (SEQ ID NO: 18), CDR2 has an amino acid sequence comprising SWAGTAWR (SEQ ID NO: 19), and CDR3 has an amino acid sequence comprising LGSYEMDHH (SEQ ID NO: 20).

In some embodiments, each X¹ is a peptide having an amino acid sequence comprising any one of SEQ ID NOS: 51-58, 61-73, 81-85, 91-95, 101-106, and 111-118. In some embodiments, each X¹ is a peptide having an amino acid sequence comprising SEQ ID NO: 55. In some embodiments, each X¹ is a peptide having an amino acid sequence comprising SEQ ID NO: 67. In some embodiments, each X¹ is a peptide having an amino acid sequence comprising SEQ ID NO: 73. In some embodiments, each X¹ is a peptide having an amino acid sequence comprising SEQ ID NO: 91.

In some embodiments, each X² is a peptide linker having an amino acid sequence comprising: AEAAAKEAAAKEAAAKAGC (SEQ ID NO: 21), AEEEKRKAEEEKRKAEEEAGC (SEQ ID NO: 22), AEEEKRKAEEEKRKAEEEKRKAEEEAGC (SEQ ID NO: 23), AEEEEKKKKEEEEKKKKAGC (SEQ ID NO: 24), AEAAAKEAAAKAGC (SEQ ID NO: 25), PSRLEEELRRRLTEGC (SEQ ID NO: 26), or AEEEEKKKQQEEEAERLRRIQEEMEKERKRREEDEERRRKEEEERRMKLEMEAKRK QEEEERKKREDDEKRKKKAGC (SEQ ID NO: 27).

In some embodiments, each X² is a peptide linker having an amino acid sequence comprising AEAAAKEAAAKEAAAKAGC (SEQ ID NO: 21).

In some embodiments, the organic linker has the structure:

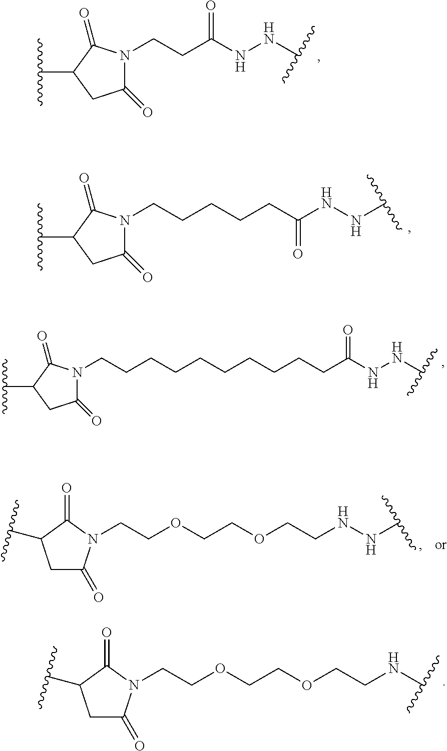

In some embodiments, the organic linker can be N-epsilon-maleimidocaproic acid hydrazide (EMCH):

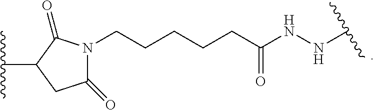 [SMILES: *NNC(=O)CCCCCN1C(=O)CC(*)C1=O]

In some embodiments, the organic linker has the structure:

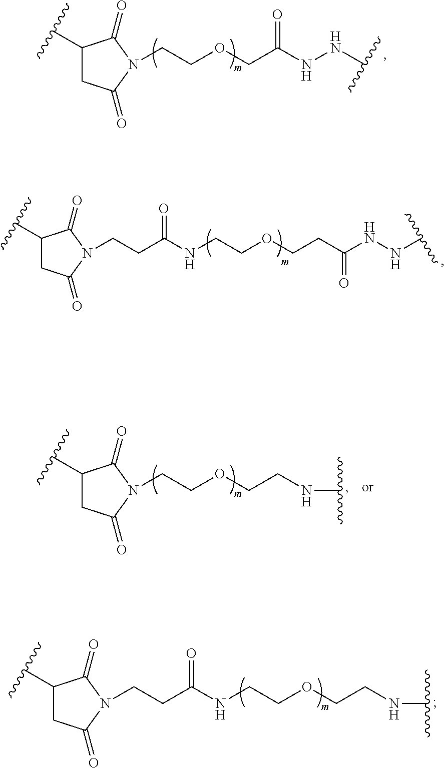

and
subscript m is an integer from 1 to 300. In some embodiments, subscript m is an integer from 1 to 100.

In some embodiments, the organic linker has the structure:

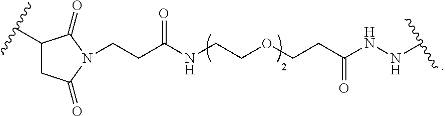

The organic linker with the above structure is known as MP2H.

In some embodiments, the random polymer of Formula III has a molecular weight of from about 0.4 MDa to about 2 MDa. In some embodiments, the random polymer of Formula III has a molecular weight of from about 0.7 MDa to about 1.5 MDa. In some embodiments, the random polymer of Formula III has a molecular weight of about 0.8 MDa.

In some embodiments, each R¹ and R² is independently C₁-C₃ alkyl or —(C₁-C₃ alkyl)-NR³R⁴. In some embodiments, each R¹ and R² is ethyl or —(CH₂)₃—NMe₂. In some embodiments, each R¹ is ethyl; and each R² is —(CH₂)₃—NMe₂. In some embodiments, each R¹ is —(CH₂)₃—NMe₂; and each R² is ethyl.

In some embodiments, each R³ and R⁴ is independently C₁-C₃ alkyl. In some embodiments, each R3 and R⁴ is methyl.

In some embodiments, subscript n is an integer of from 1 to 1500 and less than about 15% of the sum of subscripts n, p, and q; subscript p is an integer of from 1 to 1000 and less than about 10% of the sum of subscripts n, p, and q; and subscript q is an integer of from 100 to 10000. In some embodiments, subscript n is an integer of from 1 to 1000 and less than about 10% of the sum of subscripts n, p, and q; subscript p is an integer of from 1 to 800 and less than about 8% of the sum of subscripts n, p, and q; and subscript q is an integer of from 100 to 10000. In some embodiments, subscript n is an integer of from 10 to 450 and less than about 15% of the sum of subscripts n, p, and q; subscript p is an integer of from 1 to 300 and less than about 10% of the sum of subscripts n, p, and q; and subscript q is an integer of from 1000 to 3000. In some embodiments, subscript n is an integer of from 10 to 300 and less than about 10% of the sum of subscripts n, p, and q; subscript p is an integer of from 1 to 240 and less than about 8% of the sum of subscripts n, p, and q; and subscript q is an integer of from 1000 to 3000. In some embodiments, subscript n is an integer of from 10 to 300 and less than about 10% of the sum of subscripts n, p, and q; subscript p is an integer of from 1 to 60 and less than about 2% of the sum of subscripts n, p, and q; and subscript q is an integer of from 1000 to 3000. In some embodiments, subscript n is an integer of from 10 to 300 and less than about 10% of the sum of subscripts n, p, and q; subscript p is an integer of from 1 to 30 and less than about 1% of the sum of subscripts n, p, and q; and subscript q is an integer of from 1000 to 3000. In some embodiments, subscript n is an integer of from 10 to 300 and less than about 10% of the sum of subscripts n, p, and q; subscript p is an integer of from 1 to 15 and less than about 0.5% of the sum of subscripts n, p, and q; and subscript q is an integer of from 1000 to 3000.

In some embodiments, the conjugate of the present invention is a conjugate that is a random polymer of Formula III:

(X—Y—Z¹)ₙ—(Z²)ₚ—(Z³)_{q} (II)
having a molecular weight of from about 0.1 MDa to about 3 MDa; wherein
each X is independently an anti-TNF-α or anti-IL-1β peptide comprising:

(SEQ ID NO: 101)
QVQLQES GGGLVQPGGS LRLSCAASGR TFSDHSGYTY
TIGWFRQAPG KEREFVARIY WSSGNTYYAD SVKGRFAISR
DIAKNTVDLT MNNLEPEDTA VYYCAARDGI PTSRSVESYN
YWGQGTQVTV SSPSTPPTPS PSTPPGGCDD DDK,
(SEQ ID NO: 102)
QVQLQES GGGLVQPGGS LRLSCAASGR TFSDHSGYTY
TIGWFRQAPG KEREFVARIY WSSGNTYYAD SVKGRFAISR
DIAKNTVDLT MNNLEPEDTA VYYCAARDGI PTSRSVESYN
YWGQGTQVTV SSAEAAAKEA AAKEAAAKAG C,
(SEQ ID NO: 103)
QVQLQDS GGGLVQAGGS LRLSCAASGG TFSSIIMAWF
RQAPGKEREF VGAVSWSGGT TVYADSVLGR FEISRDSARK
SVYLQMNSLK PEDTAVYYCA ARPYQKYNWA SASYNVWGQG
TQVTVSSAEA AAKEAAAKEA AAKAGC,
(SEQ ID NO: 104)
QVQLQES GGGLVQAGGS LRLSCAASGG TFSSIIMAWF
RQAPGKEREF VGAVSWSGGT TVYADSVKGR FTISRDSARK
SVYLQMNSLK PEDTAVYYCA ARPYQKYNWA SASYNVWGQG
TQVTVSSAEA AAKEAAAKEA AAKAGC,
(SEQ ID NO: 105)
CGGGVDNKFN KEVGWAFGEI GALPNLNALQ FRAFIISLWD
DPSQSANLLA EAKKLNDAQA PK,
or
(SEQ ID NO: 106)
EIVMTQS PSTLSASVGD RVIITCQASQ SIDNWLSWYQ
QKPGKAPKLL IYRASTLASG VPSRFSGSGS GAEFTLTISS
LQPDDFATYY CQNTGGGVSI AFGQGTKLTV LGGGGGSGGG
GSGGGGSGGG GSEVQLVESG GGLVQPGGSL RLSCTASGFS
LSSAAMAWVR QAPGKGLEWV GIIYDSASTY YASWAKGRFT
ISRDTSKNTV YLQMNSLRAE DTAVYYCARE RAIFSGDFVL
WGQGTLVTVS SSPSTPPTPS PSTPPGGC;

each Y is an organic linker having the structure:

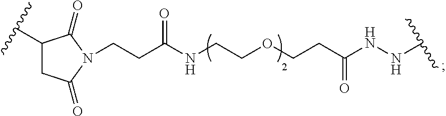

each X—Y-Z¹ moiety has the structure:

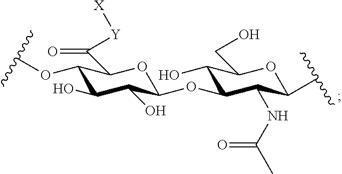

each Z² has the structure:

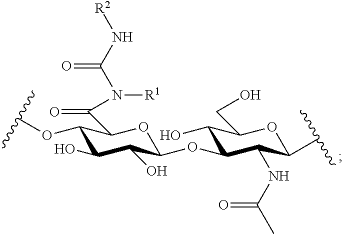

each Z³ independently has the structure:

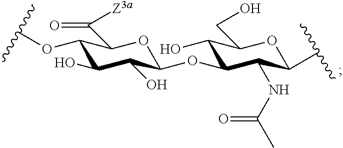

each R¹ and R² is independently C₁-C₆ alkyl, —(C₁-C₆ alkyl)-NR³R⁴, or C₅-C₅ cycloalkyl;
each R³ and R⁴ is independently H or C₁-C₆ alkyl;
each Z^{3a} is independently OH or Y′;
each Y′ has the structure

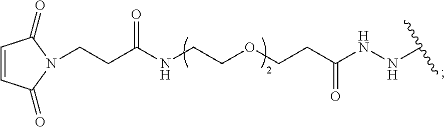

subscript n is an integer of from 1 to 1500 and less than about 15% of the sum of subscripts n, p, and q;
subscript p is an integer of from 0 to 1000 and less than about 10% of the sum of subscripts n, p, and q; and
subscript q is an integer of from 100 to 10000.

In some embodiments, the conjugate of the present invention is a conjugate that is a random polymer of Formula III:

(X—Y—Z¹)ₙ—(Z²)ₚ—(Z³)_{q} (III),
having a molecular weight of from about 0.1 MDa to about 3 MDa; wherein
each X is independently a peptide having a molecular weight of from about 5 kDa to about 200 kDa;
each Y is an organic linker;
each X—Y-Z¹ moiety has the structure:

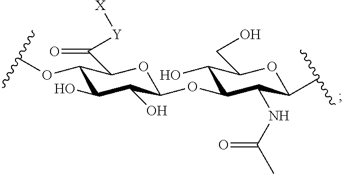

each Z² has the structure:

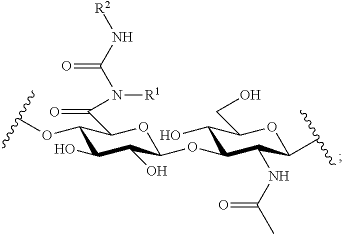

each Z³ independently has the structure:

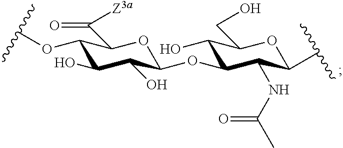

each R¹ and R² is independently C₁-C₆ alkyl, —(C₁-C₆ alkyl)-NR³R⁴, or C₅-C₅ cycloalkyl;
each R³ and R⁴ is independently H or C₁-C₆ alkyl;
each Z^{3a} is independently OH or Y′;
each Y′ is an unreacted organic linker;
subscript n is an integer of from 1 to 1500 and less than about 15% of the sum of subscripts n,
p, and q; subscript p is an integer of from 0 to 1000 and less than about 10% of the sum of subscripts n,
p, and q; and subscript q is an integer of from 100 to 10000.

In some embodiments, each X is a peptide having an amino acid sequence comprising any one of SEQ ID NOS: 51-58, 61-73, 81-85, 91-98, 101-109, 111-131, and 141-170. In some embodiments, each X is a peptide having an amino acid sequence comprising SEQ ID NO: 55. In some embodiments, each X is a peptide having an amino acid sequence comprising SEQ ID NO: 67. In some embodiments, each X is a peptide having an amino acid sequence comprising SEQ ID NO: 142. In some embodiments, each X is a peptide having an amino acid sequence comprising SEQ ID NO: 145.

In some embodiments, the conjugate is a conjugate that is a random polymer of Formula IIIa:

(X¹—X²—Y—Z¹)ₙ—(Z²)ₚ—(Z³)_{q} (IIIa),
having a molecular weight of about 0.8 MDa; wherein
each X¹ is a peptide having an amino acid sequence comprising SEQ ID NO: 55;
each X² is a peptide linker having an amino acid sequence comprising

(SEQ ID NO: 21)
AEAAAKEAAAKEAAAKAGC;

each Y is an organic linker having the structure:

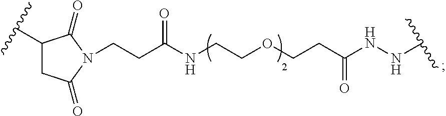

each X¹—X²—Y—Z¹ moiety has the structure:

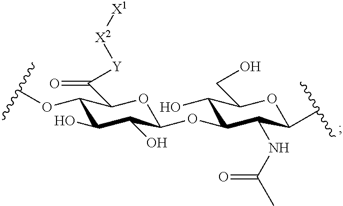

each Z² has the structure:

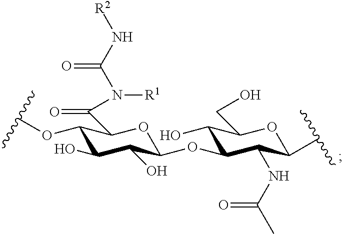

each Z³ independently has the structure:

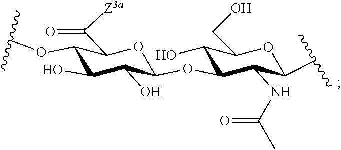

each Z^{3a} is independently OH or Y′;
each Y′ has the structure:

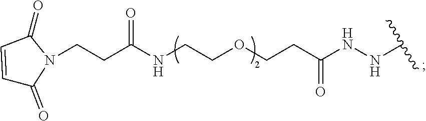

each R¹ and R² is ethyl or —(CH₂)₃—NMe₂;
subscript n is an integer of from 10 to 300 and less than about 10% of the sum of subscripts n,
p, and q; subscript p is an integer of from 1 to 15 and less than about 0.5% of the sum of subscripts n, p, and q; and
subscript q is an integer of from 1000 to 3000.

In some embodiments, the conjugate is a conjugate that is a random polymer of Formula IIIa:

(X¹—X²—Y—Z¹)ₙ—(Z²)ₚ—(Z³)_{q} (IIIa),
having a molecular weight of about 0.8 MDa;
wherein
each X¹ is a peptide having an anti-VEGF amino acid sequence comprising SEQ ID NO: 67;
each X² is a peptide linker having an amino acid sequence comprising

(SEQ ID NO: 21)
AEAAAKEAAAKEAAAKAGC;

each Y is an organic linker having the structure:

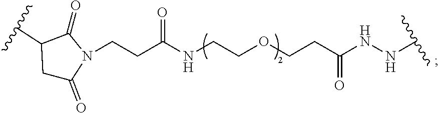

each X¹—X²—Y—Z¹ moiety has the structure:

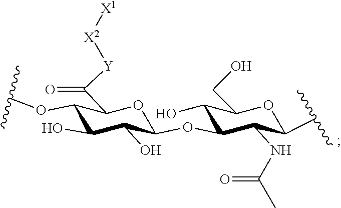

each Z² has the structure:

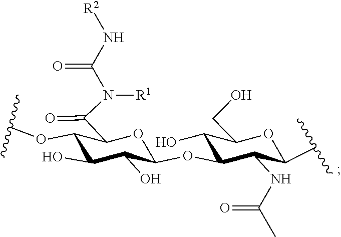

each Z³ independently has the structure:

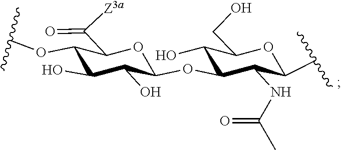

each Z^{3a} is independently OH or Y′;
each Y′ has the structure:

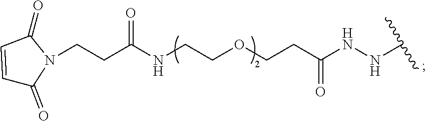

each R¹ and R² is ethyl or —(CH₂)₃—NMe₂;
subscript n is an integer of from 10 to 300 and less than about 10% of the sum of subscripts n, p, and q;
subscript p is an integer of from 1 to 15 and less than about 0.5% of the sum of subscripts n,
p, and q; and subscript q is an integer of from 1000 to 3000.

In some embodiments, a conjugate of the present invention exhibits a half-life in vivo of from about 12 hours to about 24 hours, from about 1 day to about 3 days, from about 3 days to about 7 days, from one week to about 2 weeks, from about 2 weeks to about 4 weeks, or from about 1 month to about 6 months.

In some embodiments, a conjugate of the present invention exhibits a therapeutically efficacious residence time in vivo of from about 12 hours to about 24 hours, from about 1 day to about 3 days, from about 3 days to about 7 days, from one week to about 2 weeks, from about 2 weeks to about 4 weeks, from about 1 month to about 3 months, or from about 3 months to about 6 months.

The biological activity of a conjugate is enhanced relative to the activity of the corresponding peptide in soluble form, e.g., compared to the activity of the peptide not conjugated to the polymer. In some embodiments, the biological activity of the conjugate is at least about 25%, at least about 50%, at least about 75%, at least about 2-fold, at least about 5-fold, at least about 10-fold, at least about 15-fold, at least about 20-fold, at least about 25-fold, at least about 30-fold, at least about 40-fold, at least about 50-fold, at least about 75-fold, at least about 100-fold, at least about 200-fold, at least about 500-fold, or at least about 1000-fold, or more than 1000-fold, greater than the biological activity of the peptide in soluble (unconjugated) form.

### V. Compositions

In some embodiments, the pharmaceutical composition of the present invention is a pharmaceutical composition comprising a conjugate as described herein, and a pharmaceutically acceptable excipient.

A. Formulation

For preparing pharmaceutical compositions from the conjugates of the present invention, pharmaceutically acceptable carriers can be either solid or liquid. Solid form preparations include powders, cachets, and dispersible granules. A solid carrier can be one or more substances, which may also act as diluents, binders, preservatives, disintegrating agents, or an encapsulating material. Details on techniques for formulation and administration are well described in the scientific and patent literature, see, e.g., the latest edition of Remington's Pharmaceutical Sciences, Maack Publishing Co, Easton PA (“Remington's”).

In powders, the carrier is a finely divided solid, which is in a mixture with the finely divided active component. In tablets, the active component is mixed with the carrier having the necessary binding properties in suitable proportions and compacted in the shape and size desired. The powders and tablets preferably contain from 5% or 10% to 70% of the conjugates of the present invention.

Liquid form preparations include solutions, suspensions, and emulsions, for example, water or water/propylene glycol solutions. For parenteral injection, liquid preparations can be formulated in solution in aqueous polyethylene glycol solution.

Aqueous solutions suitable for oral use can be prepared by dissolving the conjugates of the present invention in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate, one or more coloring agents, one or more flavoring agents and one or more sweetening agents, such as sucrose, aspartame or saccharin. Formulations can be adjusted for osmolality.

Also included are solid form preparations, which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

Oil suspensions can be formulated by suspending the conjugates of the present invention in a vegetable oil, such as arachis oil, olive oil, sesame oil or coconut oil, or in a mineral oil such as liquid paraffin; or a mixture of these. The oil suspensions can contain a thickening agent, such as beeswax, hard paraffin or cetyl alcohol. Sweetening agents can be added to provide a palatable oral preparation, such as glycerol, sorbitol or sucrose. These formulations can be preserved by the addition of an antioxidant such as ascorbic acid. As an example of an injectable oil vehicle, see Minto, J. Pharmacol. Exp. Ther. 281:93-102, 1997.

The pharmaceutical formulations of the invention can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent.

The compositions of the present invention can also be delivered as microspheres for slow release in the body. For example, microspheres can be formulated for administration via intradermal injection of drug-containing microspheres, which slowly release subcutaneously (see Rao, J. Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). Both transdermal and intradermal routes afford constant delivery for weeks or months.

In another embodiment, the compositions of the present invention can be formulated for parenteral administration into a body cavity such as intratumoral administration, intravitreal administration into an eye, or the intra-articular space of a joint. The formulations for administration will commonly comprise a solution of the compositions of the present invention dissolved in a pharmaceutically acceptable carrier. Among the acceptable vehicles and solvents that can be employed are water and Ringer's solution, an isotonic sodium chloride. In addition, sterile fixed oils can conventionally be employed as a solvent or suspending medium. For this purpose any bland fixed oil can be employed including synthetic mono- or diglycerides. In addition, fatty acids such as oleic acid can likewise be used in the preparation of injectables. These solutions are sterile and generally free of undesirable matter. These formulations may be sterilized by conventional, well known sterilization techniques. The formulations may contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like. The concentration of the compositions of the present invention in these formulations can vary widely, and will be selected primarily based on fluid volumes, viscosities, body weight, and the like, in accordance with the particular mode of administration selected and the patient's needs. For IV, intratumoral, or intravitreal administration, the formulation can be a sterile injectable preparation, such as a sterile injectable aqueous or oleaginous suspension. This suspension can be formulated according to the known art using those suitable dispersing or wetting agents and suspending agents. The sterile injectable preparation can also be a sterile injectable solution or suspension in a nontoxic parenterally-acceptable diluent or solvent, such as a solution of 1,3-butanediol.

In another embodiment, the formulations of the compositions of the present invention can be delivered by the use of liposomes which fuse with the cellular membrane or are endocytosed, i.e., by employing ligands attached to the liposome, or attached directly to the oligonucleotide, that bind to surface membrane protein receptors of the cell resulting in endocytosis. By using liposomes, particularly where the liposome surface carries ligands specific for target cells, or are otherwise preferentially directed to a specific organ, one can focus the delivery of the compositions of the present invention into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46: 1576-1587, 1989).

Lipid-based drug delivery systems include lipid solutions, lipid emulsions, lipid dispersions, self-emulsifying drug delivery systems (SEDDS) and self-microemulsifying drug delivery systems (SMEDDS). In particular, SEDDS and SMEDDS are isotropic mixtures of lipids, surfactants and co-surfactants that can disperse spontaneously in aqueous media and form fine emulsions (SEDDS) or microemulsions (SMEDDS). Lipids useful in the formulations of the present invention include any natural or synthetic lipids including, but not limited to, sesame seed oil, olive oil, castor oil, peanut oil, fatty acid esters, glycerol esters, Labrafil®, Labrasol®, Cremophor®, Solutol®, Tween®, Capryol®, Capmul®, Captex®, and Peceol®.

B. Administration

The conjugates and compositions of the present invention can be delivered by any suitable means, including oral, parenteral and topical methods. In some embodiments, the delivery method is intra-articular. In some embodiments, the delivery method is intravitreal. In some embodiments, the delivery method is intratumoral.

The pharmaceutical preparation is preferably in unit dosage form. In such form the preparation is subdivided into unit doses containing appropriate quantities of the conjugates and compositions of the present invention. The unit dosage form can be a packaged preparation, the package containing discrete quantities of preparation, such as packeted tablets, capsules, and powders in vials or ampoules.

The conjugates and compositions of the present invention can be co-administered with other agents. Co-administration includes administering the conjugate or composition of the present invention within 0.5, 1, 2, 4, 6, 8, 10, 12, 16, 20, or 24 hours of the other agent. Co-administration also includes administering simultaneously, approximately simultaneously (e.g., within about 1, 5, 10, 15, 20, or 30 minutes of each other), or sequentially in any order. Moreover, the conjugates and compositions of the present invention can each be administered once a day, or two, three, or more times per day so as to provide the preferred dosage level per day.

In some embodiments, co-administration can be accomplished by co-formulation, i.e., preparing a single pharmaceutical composition including the conjugates and compositions of the present invention and any other agent. Alternatively, the various components can be formulated separately.

The conjugates and compositions of the present invention, and any other agents, can be present in any suitable amount, and can depend on various factors including, but not limited to, weight and age of the subject, state of the disease, etc. Suitable dosage ranges include from about 0.1 mg to about 10,000 mg, or about 1 mg to about 1000 mg, or about 10 mg to about 750 mg, or about 25 mg to about 500 mg, or about 50 mg to about 250 mg. Suitable dosages also include about 1 mg, 5, 10, 20, 30, 40, 50, 60, 70, 80, 90, 100, 200, 300, 400, 500, 600, 700, 800, 900 or 1000 mg. The composition can also contain other compatible therapeutic agents. The conjugates described herein can be used in combination with one another, with other active agents known to be useful in modulating a glucocorticoid receptor, or with adjunctive agents that may not be effective alone, but may contribute to the efficacy of the active agent.

### VI. Methods of Treatment

In some embodiments, the present invention relates to a method and/or use comprising a conjugate or a composition as described herein for the treatment of disease or disorder in a subject in need thereof.

In some embodiments, the method comprises multiple administrations of the conjugate. In some embodiments, the method comprises administering the conjugate every day, every other day, every three days, or every week. In some embodiments, the method comprises administering the conjugate every week, every 2 weeks, every 3 weeks, or every month. In some embodiments, the method comprises administering the conjugate every month, every two months, or every three months. In some embodiments, the method comprises administering the conjugate twice or three times yearly. In some embodiments, the method comprises administering the conjugate yearly.

A. Ocular Disorder

In some embodiments, the method of the present invention is a method of treating an ocular disorder in a subject in need thereof, comprising administering to the subject a conjugate as described herein.

In some embodiments, the method comprises intravitreally administering the conjugate.

In some embodiments, the method comprises administering the conjugate every month, every two months, or every three months.

In some embodiments, the vitreous half-life of the conjugate is at least 2-fold, 3-fold, 4-fold, 5-fold, 10-fold, 20-fold, 50-fold, or at least 100-fold greater than the half-life of the unconjugated peptide. In some embodiments, the vitreous half-life of the conjugate is at least 5-fold greater than the half-life of the unconjugated peptide.

Ocular disorders that can be treated using a method of the present disclosure include, but are not limited to, uveitis, macular degeneration, also known as age-related macular degeneration (AMD), choroidal neovascularization, retinal neovascularization, proliferative vitreoretinopathy, glaucoma, and ocular inflammation. In some embodiments, the macular degeneration is wet macular degeneration. In some embodiments, the macular degeneration is dry macular degeneration.

Ocular diseases that can be treated using a method of the present disclosure include, but are not limited to, acute macular neuroretinopathy; Behcet's disease; choroidal neovascularization; diabetic uveitis; histoplasmosis; macular degeneration, such as acute macular degeneration, non-exudative age related macular degeneration and exudative age related macular degeneration; edema, such as macular edema, cystoid macular edema and diabetic macular edema; multifocal choroiditis; ocular trauma which affects a posterior ocular site or location; ocular tumors; retinal disorders, such as central retinal vein occlusion, diabetic retinopathy (including proliferative diabetic retinopathy), proliferative vitreoretinopathy (PVR), retinal arterial occlusive disease, retinal detachment, uveitic retinal disease; sympathetic ophthalmia; Vogt Koyanagi-Harada (VKH) syndrome; uveal diffusion; a posterior ocular condition caused by or influenced by an ocular laser treatment; posterior ocular conditions caused by or influenced by a photodynamic therapy; photocoagulation, radiation retinopathy; epiretinal membrane disorders; branch retinal vein occlusion; anterior ischemic optic neuropathy; non-retinopathy diabetic retinal dysfunction; retinoschisis; retinitis pigmentosa; glaucoma; Usher syndrome, cone-rod dystrophy; Stargardt disease (fundus flavimaculatus); inherited macular degeneration; chorioretinal degeneration; Leber congenital amaurosis; congenital stationary night blindness; choroideremia; Bardet-Biedl syndrome; macular telangiectasia; Leber's hereditary optic neuropathy; retinopathy of prematurity; and disorders of color vision, including achromatopsia, protanopia, deuteranopia, and tritanopia.

In some cases, the ocular disease is glaucoma, retinitis pigmentosa, macular degeneration, retinoschisis, Leber's Congenital Amaurosis, diabetic retinopathy, achromotopsia, or color blindness.

In some cases, a composition comprising a conjugate is administered by an intravitreal, transcleral, periocular, conjunctival, subtenon, intracameral, subretinal, subconjunctival, retrobulbar, or intracanalicular route of administration. In some cases, a composition comprising a conjugate is administered intravitreally. In some cases, the composition is delivered intravitreally or in close proximity to the posterior segment of the eye. In some cases, the composition is administered intravitreally by injection. In some cases, a composition comprising a conjugate is administered by intraocular injection.

B. Joint Diseases

In some embodiments, the method of the present invention is a method of treating a disease or disorder in an articular joint in a subject in need thereof, comprising administering to the subject a conjugate as described herein.

In some embodiments, the method comprises intraarticularly administering the conjugate.

In some embodiments, the intraarticular half-life of the conjugate is at least 2-fold, 3-fold, 4-fold, 5-fold, 10-fold, 20-fold, 50-fold, or at least 100-fold greater than the half-life of the unconjugated peptide. In some embodiments, the intraarticular half-life of the conjugate is at least 5-fold greater than the half-life of the unconjugated peptide.

The present invention also provides methods of treating disease and disorders of the joint tissues using the conjugates of the present invention. Examples of diseases and disorders of the joint tissues include, but are not limited to rheumatoid arthritis, wear-related osteoarthritis, age-related osteoarthritis, post-traumatic osteoarthritis, psoriatic arthritis, and aseptic implant loosening, joint effusion, ankylosing spondylitis, bursitis, gout, reactive, arthritis, synovitis, and avascular necrosis. In some embodiments, the disease or disorder is rheumatoid arthritis, wear-related osteoarthritis, age-related osteoarthritis, post-traumatic osteoarthritis, psoriatic arthritis, and aseptic implant loosening, joint effusion, ankylosing spondylitis, bursitis, gout, reactive arthritis, synovitis, or avascular necrosis.

Many polypeptides are used as drugs to attenuate immune cell function have substantial utility in treating many joint disorders. Joint tissues are particularly susceptible to injury and disease because the typical cellular responses to these assaults, i.e., upregulating of inflammatory mediators, is also a signal to encourage catabolism of articular cartilage and resorption of the underlying bone tissues. Degeneration of the articular surfaces encourages the worsening of damage to the joint tissues and further up regulation of inflammatory mediators. Over time, these mechanisms generate a feed-forward loop that results in cumulative damage to the joint tissues.

Any joint of the human or animal body can be treated using the methods and conjugates of the present invention. Representative joints include, but are not limited to, fibrous joints, cartilaginous joints, synovial joints, facet joints, synarthrosis joints, amphiarthrosis joints, and diarthrosis joints. The joints can be simple joints having two articulation surfaces, a compound joint having three or more articulation surfaces, or complex joints having two or more articulation surfaces and an articular knee or meniscus. Anatomical joints that can be treated using the conjugates and methods of the present invention include, but are not limited to, hand joints including the fingers, elbow joints, wrist joints, shoulder joints, joints of the sternum and clavicle, vertebral joints, jaw and skull joints, pelvic and hip joints, knee joints, ankle joints and foot joints including the toes. The joints can also be classified as a plane joint, ball and socket joint, hinge joint, pivot joint, condyloid joint and saddle joint. The conjugates and methods of the present invention can be used to treat the tissues of the joint, including, but not limited to, connective tissue, cartilage, articulation surfaces, synovial cavities, meniscus, and others.

Examples of drugs that are designed to attenuate immune cell function include antibodies that can interfere with Tumor Necrosis Factor-α and IL-1β, IL-6, or interferon-γ. Other examples include selective antibody inhibitors of T cell and B cell function. These antibodies may be monoclonal IgG antibodies, IgG antibody fragments, single chain scFv antibodies, single-domain heavy-chain VHH antibodies, or engineered antibody-like scaffolds such as adnectins, affibodies, anticalins, DARPins, and engineered Kunitz-type inhibitors. Other examples also include receptor decoys of immunomodulatory cytokines such as Tumor Necrosis Factor-α and IL-1β, IL-6, or interferon-γ.

One common side effect of using anti-inflammatory drugs such as those listed above is a higher risk of infection. Because they attenuate the body's immune responses, the immune system becomes impaired to fight bacteria, viruses, and parasites. Therefore, the benefits of systemic use of these drugs needs to be weighed carefully against the risks associated with systemic immune suppression. In the case of diseases where the whole body is affected by a hyperimmune disorder, such as rheumatoid arthritis, systemic use of immune attenuating drugs may be justified. However, for conditions effecting only one or a limited number of joints, the system risk of infection often does not justify the systemic use of these drugs.

As an alternative, intra-articular (IA) administration of immune modulating drugs has been proposed to prevent or inhibit the long-term effects of inflammation that are associated with osteoarthritis. However, these drugs are rapidly cleared out of the joint space and do not provide adequate duration of therapy after IA administration. After IA injection, the half-life of anti-inflammatory proteins in the synovium is short (<1.5 hours). This is evident from clinical studies where inflammation inhibitors, including infliximab and etanercept, have been administered by IA injection in humans for a variety of joint disorders. Some of these studies report a significant reduction in joint inflammation, but acknowledge that frequent (e.g. weekly) administration was required for a successful outcome. Thus, IA anti-inflammatory therapy using existing drugs would be limited by high costs and the inconvenience of frequent IA dosing. Clearly, methods to extend anti-inflammatory drug bioactivity within the synovial fluid are needed to enable this therapeutic approach for treating joint disorders.

The primary symptoms associated with joint disorders are pain, effusion, limited range of motion, and pathological remodeling of the joint anatomy. Efficacy for a treatment to treat joint disorders may include a reduction in pain as measured by a generalized assessment, such as the visual assessment score. Efficacy may also be determined based on an improved score using a system that is specific to a particular joint disorder, such as the WOMAC score for osteoarthritis, the ACR20 for rheumatoid arthritis, the Psoriatic Arthritis Quality of Life for psoriatic arthritis, or the SASSS for ankylosing spondylitis. Efficacy may also be measured using a functional output, such as an increase in pain free walking distance or an increase in the range of joint motion. Efficacy may also be measured based on radiographic evidence showing restoration of normal joint anatomy.

The conjugate can be administered at any suitable frequency or amount as discussed above. In some embodiments, the conjugate is injected into the articular joint no more than about once a month. In some embodiments, the conjugate is injected into the articular joint from about once a month to once every 6 months. In some embodiments, the conjugate is injected into the articular joint once every 2 months or once every 3 months.

1. Osteoarthritis

In 2015, an estimated 7.75 million Americans experienced symptoms of osteoarthritis (OA) that could be associated with a known joint injury. Post-traumatic OA (PTOA) accounts for at least 15% of all OA cases, although it is assumed many other OA diagnoses may also be related to a prior joint trauma. Due to a lack of disease modifying therapies, joint replacement surgery is often the only treatment option to eliminate the associated discomfort and restore mobility. However, PTOA is often diagnosed in younger patients, for whom joint replacement is not a viable option. Overall, the cost of treating these PTOA patients exceeds $4B in health care costs each year.

Short-term inhibition of injury-related inflammation will limit the long-term symptoms of PTOA. Many types of joint injury have been associated with PTOA, including dislocations, ligament tears, meniscal damage, and intra-articular fractures. Although the initial damage may be acute, the injury is sufficient to initiate a cascade of inflammatory mediators. The resulting chronic whole-joint inflammation can encourage catabolism of the articular cartilage, resulting in further tissue damage that accumulates over time and presents as PTOA. TNFα and IL-1β have well-known roles in mediating joint inflammation. These cytokines interact to promote destruction of cartilage, which occurs by both downregulating the expression of the cartilage matrix components and upregulating the expression of matrix metalloproteinases (MMPs). TNFα also stimulates osteoclast recruitment, and induces apoptosis of bone-forming osteoblasts in inflammatory environments, which contributes to the erosion of articular cartilage tissues. TNFα and IL-1β are compelling targets for mitigating the inflammatory response to joint injury. Inhibiting these key acute inflammatory cytokines in the joint environment has been proposed for early intervention to stall the progression of PTOA.

2. Inflammation Due to Immune Response to Intra-Articular Microparticles

Wear occurring between the articular surfaces of a joint can generate particles at the micron scale that drive joint inflammation and osteolysis. Wear particles may be generated due to abrasion between endogenous surfaces, such as ossified cartilage lesions, osteophytes (bone spurs), or exposed subchondral bone lesion. This type of wear particle generation occurs frequently in later stage of OA, resulting in severe joint pain and immobility. This additional inflammatory response accelerates the rate of joint tissue degeneration in OA.

Wear particles may also be formed between the surfaces of an artificial joint. In 2015, more than 7 million Americans were living with an implanted artificial joint. Nearly 250,000 of these individuals will eventually require a revision surgery due to osteolysis of the bone surrounding the device, eventually resulting in device loosening and failure.

Wear-related inflammation stems from the foreign body response to otherwise inert microparticles shed from the articulating surfaces. Macrophages inside the synovial lining readily recognize wear microparticles as foreign bodies, release pro-inflammatory factors that recruit other active immune cells to the synovium, and stimulate osteoclast expansion while simultaneously inhibiting bone formation. Thus, sustained inflammation triggers a feed-forward cycle where cartilage degeneration and osteolysis leads to more abrasions between articulating surfaces and more movement and physical stress that in turn produces more particles.

In some embodiments, the peptide modulates the activity of immune cell function. In some embodiments, the peptide inhibits tumor necrosis factor-α, interleukin-1β, interleukin-6, or interferon-γ. In some embodiments, the peptide inhibits tumor necrosis factor-α.

Tumor necrosis factor (TNFα) is a compelling target for controlling the foreign body response. TNFα has a well-known role in mediating joint inflammation. TNFα also stimulates osteoclast recruitment, and induces apoptosis of bone-forming osteoblasts in inflammatory environments, leading to osteolysis of subchondral bone. Inhibition of TNFα using a systemically-administered receptor antagonist (etanercept) has been shown to reduce bone resorption induced by wear particles in mice, although the risks associated with systemic anti-TNFα are not generally regarded as acceptable for localized conditions. As an alternative, IA anti-TNFα therapy has been proposed to prevent or inhibit the osteolytic response to intra-articular wear particle.

In some embodiments, a use of the present invention is a use of a conjugate as described herein for the preparation of a medicament for a method of treating a disease or disorder in a subject.

In some embodiments, the subject is a human.

In some embodiments, a use of the present invention is a use for treating a disease or disorder comprising a conjugate or pharmaceutical composition as described herein.

In some embodiments, a pharmaceutical composition of the present invention is a pharmaceutical composition for use in treating a disease or disorder comprising a conjugate as described herein.

In some embodiments, a conjugate of the present invention is a conjugate for use in treating a disease or disorder as described herein.

### VII. Methods of Preparation

In some embodiments, the method is a method of preparing a peptide of the present invention, comprising (a) translating a gene sequence encoding the peptide in a bacterium in a first reaction mixture; and (b) removing endotoxins from the first reaction mixture by forming a second reaction mixture from the first reaction mixture and ethylenediamine tetraacetic acid (EDTA); thereby preparing the peptide.

In some embodiments, the method is a method of preparing a peptide of the present invention, comprising (a) translating a gene sequence encoding the peptide in a bacterium in a first reaction mixture; (b) forming a second reaction mixture from the first reaction mixture and ethylenediamine tetraacetic acid (EDTA); and (c) filtering the second reaction mixture; thereby preparing the peptide. In some embodiments, the second reaction mixture further comprises sodium chloride. In some embodiments, the second reaction mixture further comprises sodium citrate. In some embodiments, the second reaction mixture further comprises sodium citrate pH 5.5.

In some embodiments, the bacterium is E. coli.

In some embodiments, the second reaction mixture comprises from about 0.1 mM to about 5 mM EDTA. In some embodiments, the second reaction mixture comprises from about 0.2 mM to about 1 mM EDTA.

Filtering the second reaction mixture can be accomplished by any method known in the art. In some embodiments, filtering the second reaction mixture comprises a filtration membrane. In some embodiments, the filtration membrane comprises polyethersulfone (PES) or regenerated cellulose. For instance, the filtration membrane can comprise a 50 kDa or 100 kDa PES membrane.

In some embodiments, a method of preparing a conjugate of the present invention comprises: (a) forming a first reaction mixture comprising a hyaluronic acid polymer having a molecular weight of from about 0.1 MDa to about 3 MDa, from about 0.1 to about 2 equivalents coupling agent per hyaluronic acid monomer, and an organic linker agent of formula H₂N—R^{Y}, wherein R^{Y} is

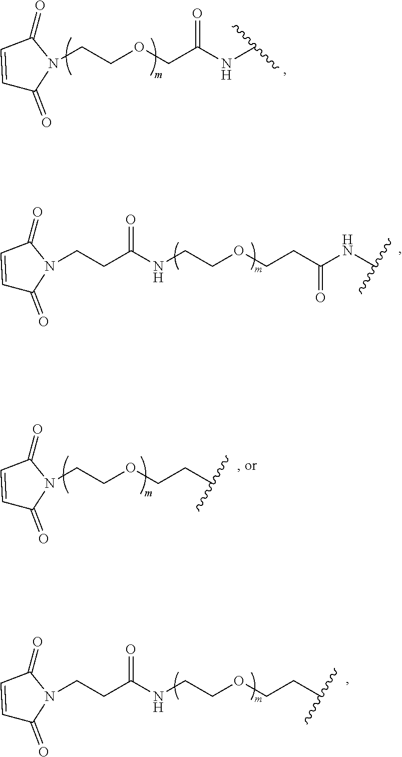

and
subscript m is an integer of from 1 to 300; thereby forming an intermediate polymer having a plurality of monomers of Formula IV:

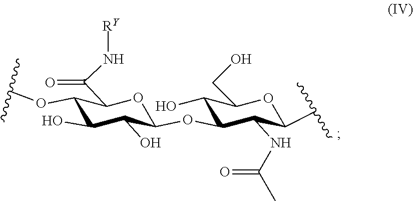

(b) forming a second reaction mixture comprising the intermediate polymer and a peptide having a molecular weight of from about 5 kDa to about 200 kDa, wherein the peptide comprises one or more —SH; thereby preparing the conjugate.

In some embodiments, the hyaluronic acid polymer has a molecular weight of from about 0.4 MDa to about 2 MDa. In some embodiments, the hyaluronic acid polymer has a molecular weight of from about 0.7 MDa to about 1.5 MDa. In some embodiments, the hyaluronic acid polymer has a molecular weight of about 0.8 MDa.

In some embodiments, the first reaction mixture comprises from about 0.2 to about 1.5 equivalents coupling agent per hyaluronic acid monomer. In some embodiments, the first reaction mixture comprises from about 0.2 to about 1 equivalent coupling agent per hyaluronic acid monomer.

In some embodiments, the coupling agent comprises a carbodiimide. In some embodiments, the coupling agent is 1-ethyl-3-(3-dimethylaminopropyl)carbodiimide, 1,3-diisopropylcarbodiimide, or dicyclohexyl carbodiimide, or a salt thereof. In some embodiments, the coupling agent is 1-ethyl-3-(3-dimethylaminopropyl)carbodiimide or a salt thereof.

In some embodiments, R^{Y} is

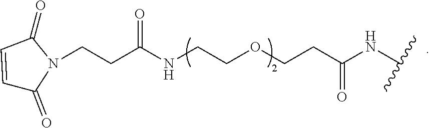

In some embodiments, the first reaction mixture comprises from about 0.2 to about 6 equivalents of the organic linker agent per hyaluronic acid monomer.

In some embodiments, the first reaction mixture comprises a catalyst. In some embodiments, the catalyst is ethyl 2-cyano-2-(hydroxyimino)acetate (Oxyma), hydroxybenzotriazole, N-hydroxysuccinimide (NHS), N-hydroxysulfosuccinimide (sulfo-NHS), or 1-hydroxy-7-azabenzotriazole, or a salt thereof. In some embodiments, the catalyst is hydroxybenzotriazole.

In some embodiments, the second reaction mixture comprises from about 0.5 to about 1.5 equivalents peptide per organic linker.

In some embodiments, a method of preparing a conjugate of the present invention comprises: (a) forming a first reaction mixture comprising a hyaluronic acid polymer having a molecular weight of about 0.8 MDa, from about 0.2 to about 1 equivalent coupling agent per hyaluronic acid monomer, and an organic linker agent of formula H₂N—R^{Y}, wherein R^{Y} is

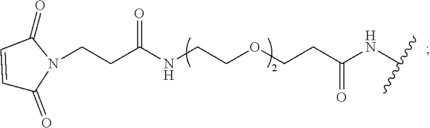

thereby forming an intermediate polymer having a plurality of monomers of Formula IV:

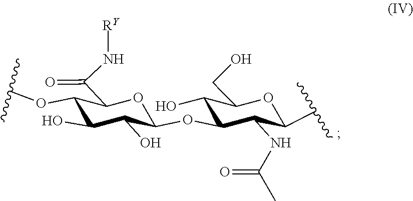

the coupling agent is 1-ethyl-3-(3-dimethylaminopropyl)carbodiimide or a salt thereof; and
the first reaction mixture comprises from about 0.2 to about 6 equivalents of the organic linker agent per hyaluronic acid monomer; and
(b) forming a second reaction mixture comprising the intermediate polymer and a peptide having a molecular weight of from about 5 kDa to about 200 kDa, wherein the peptide comprises one or more —SH; thereby preparing the conjugate.

The organic linker agent having the structure:

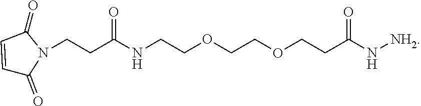 [SMILES: NNC(=O)CCOCCOCCNC(=O)CCN1C(=O)C=CC1=O]

and known as 3-(2,5-dioxo-2,5-dihydro-1H-pyrrol-1-yl)-N-(2-(2-(3-hydrazineyl-3-oxopropoxy)ethoxy)ethyl)propanamide, is also known under the abbreviation MP2H. Reference to “MP2H” as the organic linker agent or the organic linker as used herein is understood in the context of its use by one skilled in the art.

### VIII. Examples

Certain abbreviations and acronyms are used in describing the experimental details. Although most of these would be understood by one skilled in the art, the Table below contains a list of many of these abbreviations and acronyms.

TABLE 1
List of abbreviations and acronyms.

| Abbreviation | Meaning |
|---|---|
| aH | alpha-helix |
| BLI | biolayer interferometry |
| CBB | Coomassie brilliant blue |
| Da | daltons |
| DMSO | dimethyl sulfoxide |
| DMTMM | 4-(4,6-dimethoxy-1,3,5-triazin-2-yl)- |
| | 4-methyl morpholinium chloride |
| DPBS | Dulbecco's phosphate buffered saline |
| DTT | dithiothreitol |
| EDC | 1-ethyl-3-(3-dimethylaminopropyl)carbodiimide |
| EDTA | ethylenediaminetetraacetic acid |
| ELISA | enzyme-linked immunosorbent assay |
| FPLC | fast protein liquid chromatography |
| HA | hyaluronic acid |
| IL | interleukin |
| IMAC | immobilized metal affinity chromatography |
| IPTG | isopropylthiogalactoside |
| ITV | intravitreal |
| kDa | kilodaltons |
| MDa | megadaltons |
| MES | 2-(N-morpholino)ethanesulfonic acid |
| MVP | multivalent protein |
| MW | molecular weight |
| MWCO | molecular weight cutoff |
| NHS | N-hydroxysuccinimide |
| ORF | open reading frame |
| PBS | phosphate buffered saline |
| RPM | revolutions per minute |
| RT | room temperature |
| sdAb | single domain antibody |
| SDS-PAGE | sodium dodecyl sulfate polyacrylamide gel electrophoresis |
| SEC | size-exclusion chromatography |
| SEC MALS | size-exclusion chromatography multi-angle light scattering |
| TB | Terrific Broth |
| TCEP | tris(2-carboxyethyl)phosphine |

General methods were used in the Examples that follow.

### Addition of Peptide Linkers for Protein/Conjugate Stability

In frame fusions between therapeutic proteins and C-terminal peptide linkers were achieved via two methods. E. coli codon optimized nucleotides coding for peptide linkers were added to therapeutic ORFs (complete with a single C-terminal cysteine residue for MVP conjugation) and ordered as linear Geneblocks (from IDT or similar), with overhangs compatible for cloning directly into a protein expression plasmid. Alternatively, oligonucleotide primers complementary to the therapeutic ORF containing codon-optimized sequence coding for peptide linkers were extended and amplified in a PCR reaction, generating linear amplicon's that were cloned directly into protein expression plasmids. Sanger sequencing was performed on all isolated plasmids to ensure correct placement and completeness of ORF's containing therapeutics fused to peptide linkers. All ORF's were expressed via IPTG-inducible T7 promoters within commercial T7 compatible E. coli strains.

Cytoplasmic Expression in E. coli

To determine quantity of soluble protein expressed in E. coli, proteins were placed under control of an IPTG-inducible T7 promoter (NEB Shuffle T7 Express), grown to an OD_{600 nm} of 0.6 in Terrific Broth and allowed to induce for a four hour period at 37° C. with 0.5 mM IPTG. Culture sizes varied depending on need and purpose but were between 10 mL-1L.

Expression in E. coli Periplasm

For expression within E. coli periplasm, the periplasmic targeting sequence for E. coli MalE was added to the N-terminus of ORFs. All subsequent expression and downstream purification techniques were left unchanged.

### IMAC and Affinity Tag Removal

E. coli pellets from 1L culture were lysed via sonication into 25 mM HEPES, 20 mM imidazole, 400 mM sodium chloride, 0.5 mM EDTA, 5% glycerol, 0.01% Tween 20, pH 7.5, clarified at 20k*g, and applied to a GE Ni-NTA HisTrap™ column. Non-specific proteins were washed off in the buffer above containing an additional 40 mM imidazole. Proteins of interested were then eluted with a gradient to 260 mM imidazole using an FPLC. Purity was checked via SDS-PAGE and eluted peak area as identified by AKTA Unicorn software was used as a culture yield comparator. In some instances, proteins were expressed with a TEV-cleavable IMAC affinity tag at the N-terminus that was removed after IMAC purification.

### Protein A Purification

In instances where no polyhistidine affinity tag was used, a Protein A resin (JSR Life Sciences, Amsphere A3) was used to capture sdAb's from clarified E. coli lysates in 20 mM Tris, 25 mM sodium chloride, 0.5 mM EDTA, pH 8.5. Immobilized sdAb's were then washed in fresh lysate buffer, and then eluted with 50 mM sodium citrate pH 5, 25 mM NaCl, 1 mM EDTA.

### Chromatographic Polishing

For further purification of proteins, pooled IMAC eluates were diluted 5-fold with nanopure water and applied to GE HiTrap Q HP columns pre-equilibrated with 20 mM Tris, 25 mM sodium chloride, 0.5 mM EDTA, pH 8.5. These conditions were adequate for removal of contaminating E. coli proteins from the affinity chromatography eluate pool, with target proteins remaining in the column flow-through. Q column flow-through was further diluted two-fold with nanopure water, pH'd to 5 with acetic acid, and applied to GE HiTrap SP HP columns pre-equilibrated with 10 mM sodium citrate, 0.25 mM EDTA, pH 5.0. Purified proteins were eluted with a gradient to 25 mM sodium citrate, 0.5 M sodium chloride, 1 mM EDTA, pH 5.5. Purity was confirmed via SDS-PAGE and eluted peak area as identified by AKTA Unicorn software was used as a protein yield comparator.

### Endotoxin Removal and Protein Finishing

Pure SP elution fractions were pooled, and material was passed twice through 100 kDa regenerated cellulose spin concentrators to remove endotoxins. 100 kDa spin concentrator flow-through protein solutions were then concentrated on 3 kDa regenerated cellulose spin concentrators to a protein concentration >175 mg/mL. Sterile glycerol was then added to 10% C_{F} (v/v) and then flash frozen and stored at −80° C.

Alternatively, after Q anion exchange chromatography, a final concentration/purification step was performed with cation exchange chromatography. Proteins were bound at pH 5, and eluted over a gradient from solution A (10 mM sodium citrate pH 5, 0.25 mM EDTA) to solution B (25 mM sodium citrate pH 5.5, 1M NaCl, 1 mM EDTA), typically eluting between 10 and 25% B. Peak fractions were then pooled, and these pooled protein solutions were then passed through 100 kDa filter membranes (either PES, or regenerated cellulose) at 3000*g. Pure protein was then further concentrated on 10 kDa filter membranes.

### Free Cysteine Gel Analysis

To ensure a single free cysteine was available for conjugation to biopolymers, ˜20 equivalents of a 1.2 kDa PEG-maleimide moiety were incubated at 42° C. for 45 minutes and run on 4-20% SDS-PAGE to confirm mobility is shifted by a single 1.2 kDa gel mobility shift.

### Additional Protein/Conjuyate Stabilizing Mutations

Protein sequences were retrieved via BLAST query against the PDB database. MSA manipulation was performed with the Jalview program including sequence alignment using the Clustal Omega, manual curation of sequences from the alignment to include only sdAb's with the desired topology, and removal of sequence redundancy such that only ˜100 sequences remained within the MSA. Positions within the MSA containing conservation scores of nine or above were considered consensus and incorporated into sdAb sequences containing c-terminal alpha helical linker peptides between the ORF and the conjugation cysteine.

### Site-Directed Mutagenesis

Complimentary oligonucleotide pairs containing desired codon-optimized amino acid substitution mutations were designed following guidelines published within the Agilent QuikChange Site-directed mutagenesis kit protocols and purchased from IDT. SDM PCR reactions were performed on ˜10 ng of plasmid DNA according to manufacturer's protocols. Newly isolated plasmids were subjected to Sanger sequencing to confirm proper amino acid substitution(s).

### Thermal Precipitation of Soluble Lysate Protein

25× volumes of lysis buffer 25 mM HEPES, 20 mM imidazole, 400 mM sodium chloride, 0.5 mM EDTA, 5% glycerol, 0.01% Tween 20, pH 7.5 were added to E. coli pellets harvested from 25 mL TB culture, and sonicated 5× on ice at 40% output using a probe sonicator. Once lysed, whole cell extracts were clarified at 10K*g and 100 uL of supernatant were aliquoted and subjected to incubations at 50° C., 60° C., 70° C., and 80° C. for 15 minutes, followed by a ten-minute incubation on ice. Thermally precipitated proteins were removed at 10K*g for five minutes, and soluble extract fractions were combined directly with Laemmli sample buffer, denatured, and run on 4-20% SDS-PAGE to evaluate yield and stability.

### Example 1. Peptide Linker Evaluation

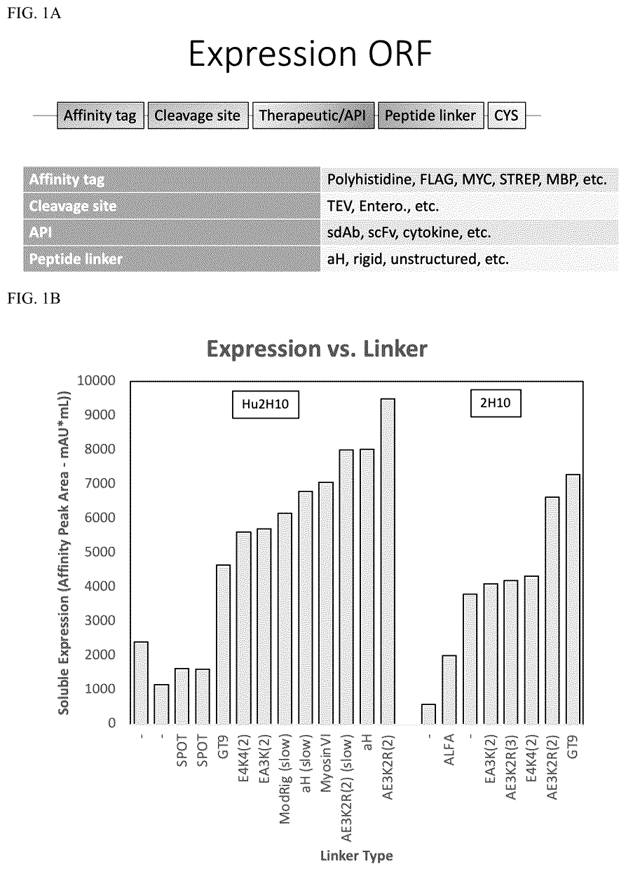

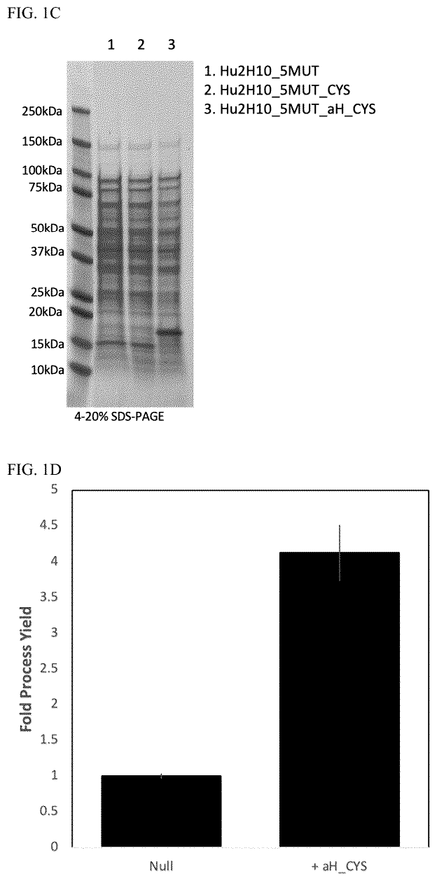

Prior to the attachment to the polymer, the peptide, containing a biologically active peptide of interest attached to a peptide linker, was expressed in E. coli, based on the expression open reading frame (ORF) in FIG. 1A. Various peptide linkers connecting the biologically active peptide to the polymer were evaluated (Table 2).

TABLE 2
Peptide Linkers

| Linker Name | Linker Type | Linker Sequence |
|---|---|---|
| aH | α-helical | AEAAAKEAAAKEAAAKAGC (SEQ ID NO: 21) |
| AE3K2R(2) | α-helical | AEEEKRKAEEEKRKAEEEAGC (SEQ ID NO: 22) |
| AE3K2R(3) | α-helical | AEEEKRKAEEEKRKAEEEKRKAEEEAGC (SEQ ID NO: 23) |
| E4K4(2) | α-helical | AEEEEKKKKEEEEKKKKAGC (SEQ ID NO: 24) |
| EA3K(2) | α-helical | AEAAAKEAAAKAGC (SEQ ID NO: 25) |
| Alfa | α-helical | PSRLEEELRRRLTEGC (SEQ ID NO: 26) |
| Myosin VI | α-helical | AEEEEKKKQQEEEAERLRRIQEEMEKERKRREED |
| | | EERRRKEEEERRMKLEMEAKRKQEEEERKKRED |
| | | DEKRKKKAGC (SEQ ID NO: 27) |
| Spot | Unstructured | PDRVRAVSHWSSC (SEQ ID NO: 28) |
| GT9 | Unstructured | GTGTGTGTGTGTGTGTGTGC (SEQ ID NO: 29) |
| Modified | Rigid | TPTTPPTPTPGTPPGGC (SEQ ID NO: 30) |
| Rigid | | |

FIG. 1B shows the soluble expression of exemplary protein 2H10 in E. coli. An improvement of yield in soluble fraction was observed with 2H10 variants containing alpha-helical peptide linkers. Presence of the alpha-helical peptide linker improved expression when reactive cysteines were present at the C-terminus of the protein.

FIG. 1C shows that the increase in soluble expression is evident in SDS-PAGE analysis of comparable peptides. Cultures containing autoinduction media and carbenicillin (5 mL) were grown to saturation overnight at 37° C. Saturated cultures were collected by centrifugation, washed with 1 mL PBS, and collected by repeat centrifugation. Supernatants were aspirated and cultures frozen at −80° C. Cells were lysed via probe sonicator on ice, cleared with centrifugation, normalized to protein content and run on 4-20% SDS-PAGE. The protein expression of Hu2H10_5MUT (SEQ ID NO: 55) and Hu2H10_5MUT_CYS (SEQ ID NO: 141) as evidenced by the band around 15 kDa was lower than for Hu2H10_5MUT_aH_CYS (SEQ ID NO: 142) containing the alpha-helical peptide linker of SEQ ID NO: 21 at the C-terminus (band at around 17 kDa).

The enhanced soluble expression was not limited to the above proteins. The anti-VEGF protein HuNb42 also showed an increase in soluble expression with addition of a C-terminal alpha-helical peptide. FIG. 1D shows soluble expression of HuNb42_A88P (SEQ ID NO: 67) (“null”) as compared with HuNb42_A88P aH_Cys (SEQ ID NO: 145) (“+aH_CYS”). The total process yield per liter culture media with the alpha-helical linker was about four-fold higher than that for the corresponding protein without the C-terminal alpha-helical peptide.

### Example 2. Humanness Effect on Soluble Expression

A higher degree of humanness is desirable for therapeutic peptides and proteins to decrease risk of immunogenicity. However, certain residues within single domain antibodies impacted humanness while simultaneously decreased stability. Accordingly, a systematic evaluation was performed for specific point mutations in the framework regions as related to humanness and stability.

Sequence humanization was performed using computational resources from Abysis antibody analyzer and the T20 score analyzer from LakePharma. For sdAb's targeting human proteins, certain amino acids within the consensus sequence were changed such that a T20 framework-only score of 85 or greater was achieved.

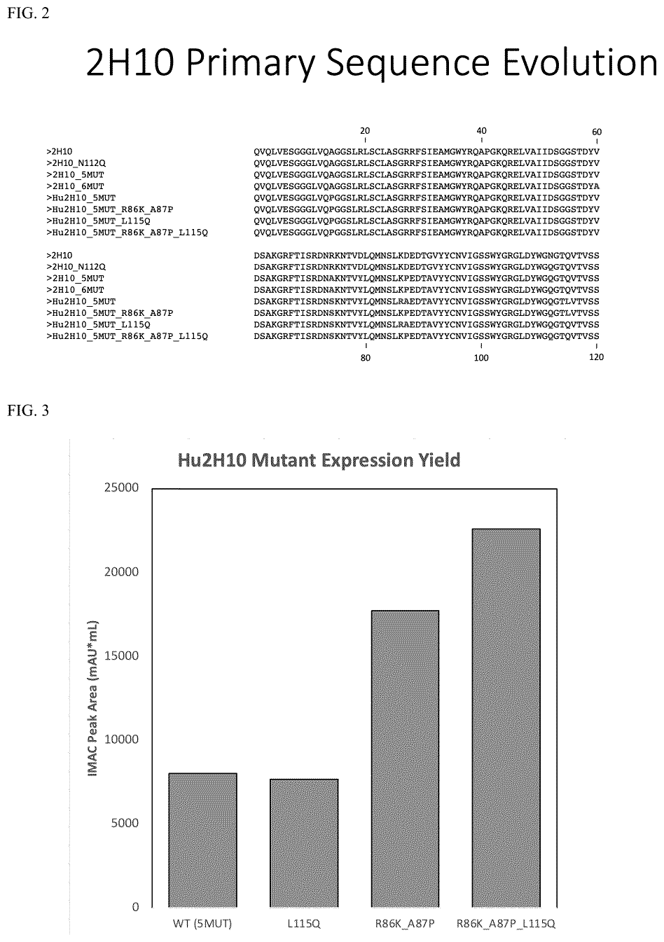

FIG. 2 shows the amino acid sequences of 2H10 and point mutation variants tested for expression in E. coli. As illustrated in FIG. 3, variants Hu2H10 R86K A87P (SEQ ID NO: 56) and Hu2H10 R86K A87P L1 15Q (SEQ ID NO: 58) had higher protein expression than Hu2H10 5MUT (SEQ ID NO: 55) or Hu2H10 L115Q (SEQ ID NO: 57).

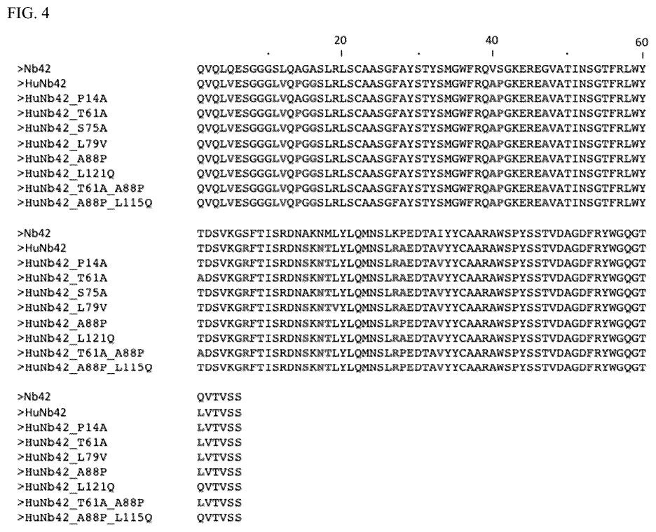

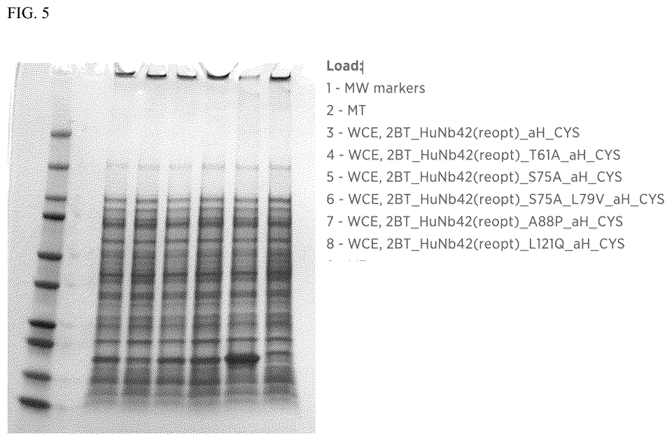

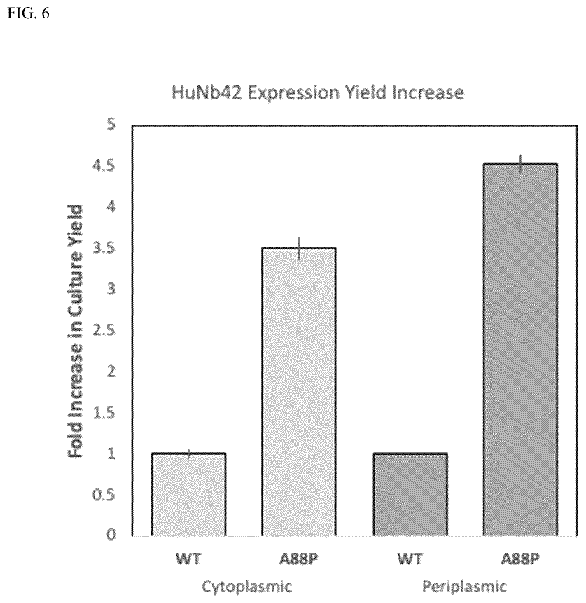

FIG. 4 shows the amino acid sequences of Nb42 and point mutation variants tested for expression in E. coli. FIG. 5 shows that the HuNb42 A88P variant (SEQ ID NO:67) exhibited increased soluble expression. FIG. 6 shows that the higher relative expression did not depend on cell compartment, as both cytoplasmic and periplasmic compartments showed higher relative levels of soluble expression.

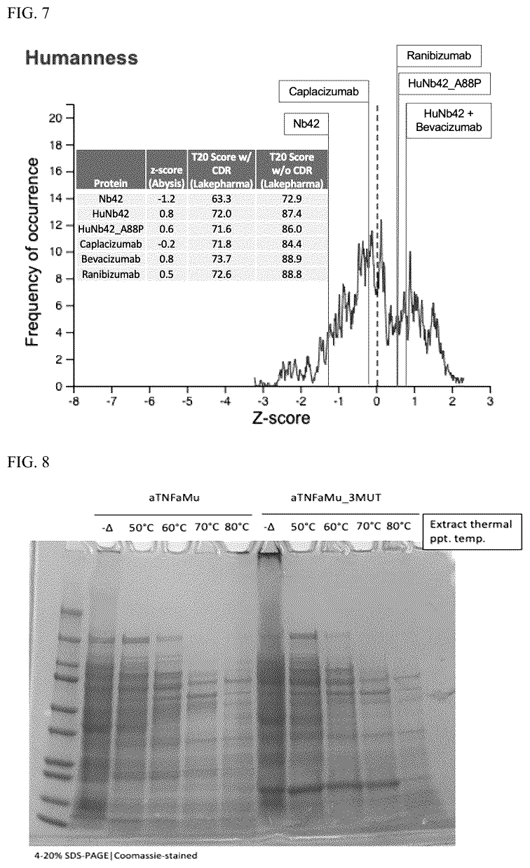

FIG. 7 shows the humanness of Nb42 (SEQ ID NO:61), HuNb42 (SEQ ID NO:62), and HuNb42 A88P variant (SEQ ID NO:67) as compared to caplacizumab, bevacizumab, and ranibizumab. HuNb42 (SEQ ID NO:62) and HuNb42 A88P variant (SEQ ID NO:67) were each comparable to the literature humanized antibodies in terms of humanness as measured by Z-score or T20 score.

FIG. 8 shows Coomassie brilliant blue (CBB) staining of E. coli cell extracts that expressed aTNFaMu (SEQ ID NO: 71) or aTNFaMu_3MUT (SEQ ID NO: 72) at RT, 50° C., 60° C., 70° C., and 80° C. The stain illustrates that the three point mutations within aTNFaMu increased yield and thermal stability of the protein up to about 70° C.

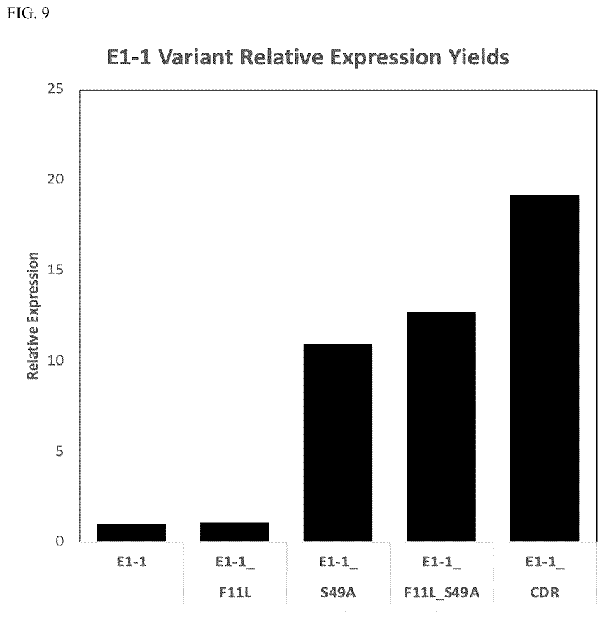

FIG. 9 shows the effect of specific point mutations in E1-1 on protein expression yields. E1-1 S49A (SEQ ID NO: 83), E1-1 F11L S49A (SEQ ID NO: 84), and E1-1 CDR (SEQ ID NO: 85) showed about 10 fold or greater relative protein expression compared to E1-1 (SEQ ID NO: 81) or E1-1 F11L (SEQ ID NO: 82).

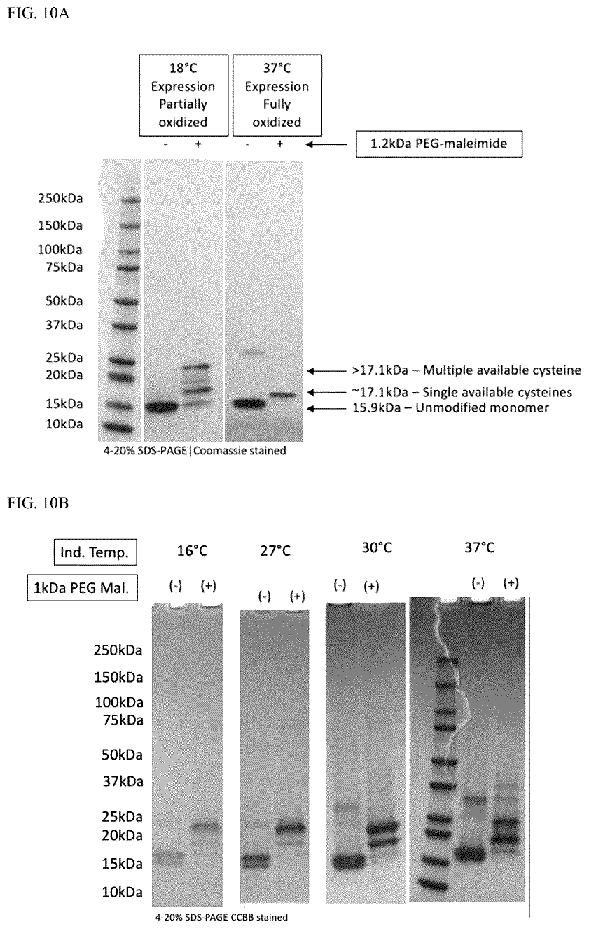

FIG. 10 shows gel shift assays consistent with fewer reactive cysteines within the final protein preparations when protein synthesis was performed at higher temperature (upper graph). Fewer reactive cysteines would indicate naturally occurring disulfide bridges were forming within the antibodies, thus yielding a more stable product. A similar gel shift (lower graph) showed gradient of disulfide bridge formation from low to high when temperature of protein synthesis was raised.

### Example 3. Endotoxin Removal

Endotoxin is not desired in protein preps as it carries through to drug conjugation steps and is source of contamination in animal testing (endotoxins cause immune response).

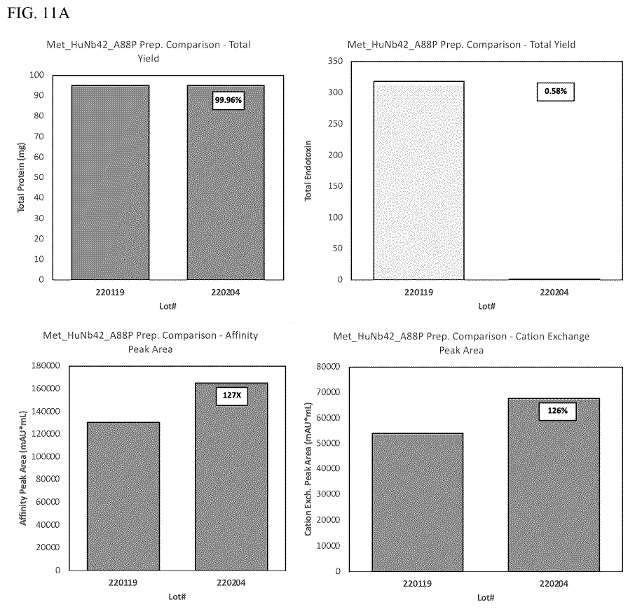

Endotoxin removal was dependent on presence of EDTA within buffers, causing endotoxins to aggregate to a certain size and become filterable while minimizing protein losses during filtration. FIG. 11A-11B show that the endotoxin removal processes removed >99.5% of the endotoxin in samples.

FIG. 11B shows removal of endotoxin with 50 and 100 kDa filters and recovery of anti-TNFα 3MUT VHH (mouse)-aH (SEQ ID NO: 104) content of same. Method: Protein solution at ˜15 mg/mL in 25 mM sodium citrate pH 5.5, 100 mM NaCl, 1 mM EDTA was passed through 50 and 100 kDa polyethersulfone membrane (PES) filters at 15K*g for 10 minutes at room temperature. Protein concentrations were measured using A280 spectrophotometry (nanodrop) and endotoxin was measured using Charles River Endosafe LAL cartridges.

### Method:

5 mL cultures were grown in TB-autoinduction media+antibiotic to saturation at 37° C. Cells were collected via centrifugation at 4000 RPM for 10′ at 4° C. Pellets were washed with 1 mL PBS and transferred to Eppendorf tubes. Cells were pelleted at 14000 RPM, 2.5′ 4° C. and supernatants were aspirated and frozen. Frozen cell pellets were thawed on ice, and sonicated on ice in lysis buffer (50 mM HEPES pH 7.5, 20 mM imidazole, 400 mM NaCl, 5% glycerol, 0.01% tween-20, and 0.5 mM EDTA) using small tip sonicator, 5″ pulse on, 5″ pulse off for 60″ total at 40% output. Cell lysates were normalized to total protein content using Nanodrop A280, and run on 4-20% SDS-PAGE under denaturing and reducing conditions. Gels were stained in InstaBlue protein stain, and destained extensively in water. % densitometry signal was calculated using ImageJ software and normalized across entire gel lanes. Overexpression was achieved when >10% of total lane protein signal was due to band at approximate predicted molecular weight, and not seen in uninduced sample controls. An illustrative SDS-PAGE gel is shown in FIG. 11C.

A summary of expression densitometry measurements for certain peptides of the present invention is shown in Table 3 below.

TABLE 3
Expression Densitometry Values

| Name | Sequences^{§} | % of Total |
|---|---|---|
| aVegF_VHH | SEQ ID NO: 91 | 21.3 |
| aVegF_VHH_3MUT | SEQ ID NO: 96 | 21.9 |
| aVegF_VHH_5MUT | SEQ ID NO: 97 | 20.9 |
| aVegF_VHH_6MUT | SEQ ID NO: 98 | 18.3 |
| aTNFaHu | SEQ ID NO: 102* | 28.3 |
| aTNFaMu_3MUT | SEQ ID NO: 104* | 16.9 |
| aTNFaHu_7MUT | SEQ ID NO: 107 | 25.0 |
| Hu_aTNFaMu_3MUT | SEQ ID NO: 108 | 12.8 |
| Hu_aTNFaMu_5MUT | SEQ ID NO: 109 | 17.8 |
| Hu_aEGFR_5MUT | SEQ ID NO: 119 | 11.8 |
| aAng2_D4 | SEQ ID NO: 120 | 30.0 |
| Hu_aAng2_D4_3MUT | SEQ ID NO: 121 | 23.7 |
| aAn2_H4 | SEQ ID NO: 122 | 25.5 |
| Hu_aAng2_H4_6MUT | SEQ ID NO: 123 | 10.7 |
| aAng2_0027 | SEQ ID NO: 124 | 34.9 |
| Hu_aAng2_0027_2MUT | SEQ ID NO: 125 | 19.8 |
| aEpCam_Nb4 | SEQ ID NO: 126 | 15.4 |
| Hu_aEpCAM_Nb4_4MUT | SEQ ID NO: 127 | 16.1 |
| aEpCam_Nb5 | SEQ ID NO: 128 | 15.2 |
| Hu_aEpCAM_Nb5_11MUT | SEQ ID NO: 129 | 17.9 |
| aEpCAM_Nb22 | SEQ ID NO: 130 | 11.9 |
| Hu_aEpCAM_Nb22_6MUT | SEQ ID NO: 131 | 14.9 |
| aPDL1_Nb1 | SEQ ID NO: 155 | 28.6 |
| Hu_aPDL1_Nb1_4MUT | SEQ ID NO: 156 | 19.9 |
| aPDL1_Nb104E10 | SEQ ID NO: 157 | 25.0 |
| Hu_aPDL1_Nb104E10_1MUT | SEQ ID NO: 158 | 23.4 |
| aPDL1_NbG5 | SEQ ID NO: 159 | 19.6 |
| Hu_aPDL1_NbG5_4MUT | SEQ ID NO: 160 | 27.5 |
| aPDL2_103E4 | SEQ ID NO: 161 | 25.8 |
| Hu_aPDL2_103E4_2MUT | SEQ ID NO: 162 | 23.4 |
| aPDL2_103E5 | SEQ ID NO: 163 | 16.2 |
| Hu_aPDL2_103E5_4MUT | SEQ ID NO: 164 | 18.7 |
| aPDL2_Nb103F10 | SEQ ID NO: 165 | 17.8 |
| Hu_aPDL2_Nb103F10_5MUT | SEQ ID NO: 166 | 22.2 |
| Hu_aPD1_102C3_3MUT | SEQ ID NO: 113 | 28.4 |
| Hu_aPD1_102C12_3MUT | SEQ ID NO: 114 | 22.2 |
| Hu_aPD1_102E2_3MUT | SEQ ID NO: 115 | 23.3 |
| Hu_aPD1_102E8_3MUT | SEQ ID NO: 116 | 29.1 |
| Hu_aPD1_102H12_4MUT | SEQ ID NO: 117 | 22.6 |
| Hu_aIL-1B_5C_5MUT | SEQ ID NO: 167 | 17.4 |
| Hu_aIL-1B_6C_3MUT | SEQ ID NO: 168 | 20.7 |
| Hu_aIL-1B_11H_3MUT | SEQ ID NO: 169 | 13.3 |
| Hu_aIL-1B_12D_2MUT | SEQ ID NO: 170 | 23.3 |

^{§}Each sequence listed was covalently attached to a C-terminal alpha-helical peptide of SEQ ID NO: 21 except where indicated with asterisk (“*”).

### Example 4. Consensus Sequence for Protein Expression of Single Domain Antibodies

Based on the data presented in the Examples above, the following framework sequence permits single domain antibodies to be expressed more stably and/or be more human-like. Table 4 and Table 5 show an exemplary framework region with permissible amino acid substitutions.

TABLE 4
Framework Region

| Region | Length | Sequence |
|---|---|---|
| Framework | 26 | QVQLVESGGGLVQPGGSLRLSCAASG (SEQ ID NO: 5) |
| 1 | | |
| CDR1 | variable, for example, 7 amino acids | |
| Framework | 18 | MGWFRQAPGKEREFVAAI (SEQ ID NO: 6) |
| 2 | | |
| CDR2 | variable, for example, 8 amino acids | |
| Framework | 39 | YADSVKGRFTISRDNSKNTVYLQMNSLRPEDTAVYYCAA |
| 3 | | (SEQ ID NO: 7) |
| CDR3 | variable, for example, 18 amino acids | |
| Framework | 12 | YWGQGTLVTVSS (SEQ ID NO: 8) |
| 4 | | |

TABLE 5
Permissible Substitutions on Exemplary Peptide

| | Consensus | Permissible |
|---|---|---|
| Position | amino acid | substitutions |
| 1 | Q | E, D |
| 2 | V | — |
| 3 | Q | — |
| 4 | L | — |
| 5 | V | Q, A, E |
| 6 | E | — |
| 7 | S | T |
| 8 | G | — |
| 9 | G | — |
| 10 | G | — |
| 11 | L | S, V |
| 12 | V | A |
| 13 | Q | — |
| 14 | P | A, T |
| 15 | G | — |
| 16 | G | D, R |
| 17 | S | — |
| 18 | L | — |
| 19 | R | — |
| 20 | L | — |
| 21 | S | — |
| 22 | C | — |
| 23 | A | V, T, E |
| 24 | A | V |
| 25 | S | — |
| 26 | G | — |
| 27-33 | CDR1 (XXXXXXX) | — |
| 34 | M | I, V, L |
| 35 | G | S, A |
| 36 | W | — |
| 37 | F | Y, V |
| 38 | R | — |
| 39 | Q | — |
| 40 | A | V, P, T |
| 41 | P | — |
| 42 | G | — |
| 43 | K | — |
| 44 | E | G, A, Q |
| 45 | R | L |
| 46 | E | — |
| 47 | F | G, W, L |
| 48 | V | — |
| 49 | A | G, S |
| 50 | A | S, G |
| 51 | I | — |
| 52-59 | CDR2 (XXXXXXXX) | |
| 60 | Y | — |
| 61 | A | G, S, T |
| 62 | D | — |
| 63 | S | — |
| 64 | V | — |
| 65 | K | — |
| 66 | G | — |
| 67 | R | — |
| 68 | F | — |
| 69 | T | — |
| 70 | I | — |
| 71 | S | — |
| 72 | R | Q |
| 73 | D | — |
| 74 | N | S, D |
| 75 | S | A, D |
| 76 | K | — |
| 77 | N | K |
| 78 | T | M |
| 79 | V | |
| 80 | Y | D, S |
| 81 | L | — |
| 82 | Q | — |
| 83 | M | — |
| 84 | N | D |
| 85 | S | N |
| 86 | L | — |
| 87 | R | — |
| 88 | P | A |
| 89 | E | — |
| 90 | D | — |
| 91 | T | — |
| 92 | A | — |
| 93 | V | M, L, I |
| 94 | Y | — |
| 95 | Y | — |
| 96 | C | — |
| 97 | A | |
| 98 | A | |
| 99-116 | CDR3 (XXXXXXXXXXXXXXXXXX) | |
| 117 | Y | |
| 118 | W | — |
| 119 | G | — |
| 120 | Q | K |
| 121 | G | — |
| 122 | T | — |
| 123 | L | Q |
| 124 | V | — |
| 125 | T | — |
| 126 | V | — |
| 127 | S | — |
| 128 | S | — |

### Example 5. Preparation of Purified Thiol Reactive Hyaluronic Acid Conjugate Intermediates

Sodium hyaluronate (HA, 830 kDa) was suspended in water or 0.1 M 2-(N-morpholino)ethanesulfonic acid buffer pH 5.7 at 4 mg/mL by gentle rotation or mixing with nutation overnight at RT. To 3 mg (3.6 nmol, amount will vary based on polymer composition and MW) of HA in solution is added hydroxybenzotriazole (HOBt) hydrate as a ˜5-100 mg/mL stock solution in DMSO, thiol reactive linker agent (e.g., hydrazide-X-thiol-reactive-group such as MP2H) in 10-100% DMSO (10-100 mg/mL stock), and a coupling agent (1-ethyl-3-(3-dimethylaminopropyl)carbodiimide hydrochloride (EDC)) in 0.1 M MES buffer pH 5.7. The molar equivalents for each reactant per mole of HA and per carboxylate for different methods of performing the reaction, and example methods are described in the Table 6 and Table 7 below:

TABLE 6
Relative Ratios of Coupling Agent,
Catalyst, and Linkers in Methods

| Reactant | Method 1 | Method 2 | Method 5* |
|---|---|---|---|
| Coupling agent (EDC) | 500-750 | 1000-1500 | 9500-12000 |
| HOBt | 3000 | 2000 | 50 |
| Linker agent MP2H | 3000 | 1500 | 500-1000 |

EDC range reaction set up using molar equivalents per mole of 830 kDa HA.
*EMCH used instead of MP2H in the specific intermediates where indicated.

TABLE 7
Ratios of Coupling Agent, Catalyst, and Linkers
per Polymer Carboxylate in Methods

| Reactant | Method 1 | Method 2 | Method 5* |
|---|---|---|---|
| Coupling agent (EDC) | 0.25-0.375 | 0.5-0.75 | 4.75-6 |
| Catalyst (HOBt) | 1.5 | 1 | 0.025 |
| Linker agent MP2H | 1.5 | 0.75 | 0.25-0.5 |

Equivalents of reactants per HA monomer (~2000 carboxylates per 830 kDa HA).
*EMCH used instead of MP2H in the specific intermediates where indicated.

Solution was mixed with gentle pipetting between each reagent addition and the final reaction volume was raised to 1 mL with buffer. The final mixture was allowed to react at room temperature for 45 min to 2 h with nutating mixer depending on Method. After the reaction, the thiol reactive biopolymer was purified using 7 kDa MWCO 5-10 mL Zeba desalting spin column equilibrated with 10% v/v glycerol (optional) pH 6.5 DPBS, and 0.01% v/v polysorbate 20 (optional), loaded with crude reaction at 20% volume of resin. The desired intermediate was eluted into clean conical tube using centrifuge at RT, elution time ˜25-60 minutes. The intermediate was used immediately for reaction with thiol or aliquotted and flash frozen on dry ice. Maleimide concentration and number of modifications per polymer was determined using UV absorbance, NMR, or a modified Ellman's reaction assay.

Alternatively, reaction pH or equivalents of hydrazide linker, catalyst, and coupling agent (EDC) were altered higher or lower to increase or decrease the number of thiol reactive small molecule linkers covalently linked per biopolymer (valency).

Alternative coupling reagents can be used in place of EDC and HOBt such as DMTMM or oxyma. Activated biopolymer intermediate can also be purified away from reactants using size exclusion chromatography, other desalting columns, tangential flow filtration, ion exchange chromatography, dialysis, or alcohol/acetone precipitation.

After purification, a UV spectrum (200-324 nm) was taken for intermediates prepared using different methods on a BioTek Synergy plate reader using a Take3 microspot plate. Maleimide concentration can be determined by absorbance at 230 nm, or by comparing spectra to a reference standard intermediate.

NMR analysis of conjugates was performed at the Complex Carbohydrate Research Center (CCRC) at University of Georgia using at 25° C. on a Bruker Advance III spectrometer (¹H, 600.13 MHz) equipped with a 5 mm cryoprobe. After standard preparation of intermediate using Method 1 and Method 5 at a 6 mL scale, the intermediate was purified into HPLC grade water using desalting resin and shipped to the CCRC on wet ice. The samples were left at 4° C. for several weeks resulting in partial maleimide hydrolysis observed in the NMR spectra. For NMR sample prep, 0.7 ml of intermediate stock solutions (2.9 mg/ml) were pipetted into 7-ml screw cap tubes. 1.3 ml of % D₂O (99.9)) was added to each sample and thoroughly mixed by vortex. The samples were then dried using a SpeedVac vacuum concentrator at room temperature. The dried samples were then redissolved in 700 μL D₂O (99.98%) for NMR analysis.

Chemical analysis of example reaction products are described in Table 8 below.

TABLE 8
Exemplary Intermediates Prepared
Using a Method Described Above

| | | | | N-acylurea |
|---|---|---|---|---|
| | | Maleimide | Maleimide | Substitution |
| | Intermediate | valency | valency* | (by ¹H NMR, |
| Method | number | (NMR) | (Ellman's) | mole %) |
| 1 | 1-1 | 203 | 128 | Undetectable |
| 2 | 2-4 | ND | 128 | ND |
| 5 | 5-1 | 118 | 123 | 8.2% |

ND = not determined,
*= determined the day the samples were received by CCRC

Tabular representations of intermediates synthesized using the three different methods are shown in Table 9, Table 10, and Table 11 below with their resulting maleimide concentration, valency, and reaction efficiency based on HA monomer.

TABLE 9
Method 1 Intermediates

| | | | Equiv. | | Maleimide | Reaction |
|---|---|---|---|---|---|---|
| | Equiv. | Equiv. | coupling | | Valency | efficiency |
| | catalyst | linker | agent | [Maleimide] | assuming | based on |
| Int. # | (HOBt) | (MP2H) | (EDC) | (μM) | no HA loss | HA-COOH |
| 1-1 | 3000 | 3000 | 500 | 483.9 | 128 | 6.2% |
| 1-2 | 3000 | 3000 | 500 | 350.14 | 124 | 6.0% |
| 1-3 | 3000 | 3000 | 500 | 422.26 | 141 | 6.8% |
| 1-4 | 3000 | 3000 | 500 | 393.05 | 139 | 6.7% |
| 1-5 | 3000 | 3000 | 500 | 397.97 | 136 | 6.6% |
| 1-6 | 3000 | 3000 | 500 | 334.1 | 115 | 5.6% |
| 1-7 | 3000 | 3000 | 750 | 429.11 | 145 | 7.0% |
| 1-8 | 3000 | 3000 | 500 | 361.2 | 136 | 6.6% |
| 1-9 | 2000 | 3000 | 500 | 306 | 97 | 4.7% |
| 1-10 | 1000 | 2000 | 500 | 104 | 34 | 1.6% |
| 1-11 | 1000 | 2500 | 500 | 150 | 47 | 2.3% |
| 1-12 | 1500 | 3000 | 750 | 355 | 112 | 5.4% |
| 1-13 | 3000 | 3000 | 500 | 484 | 137 | 6.6% |
| 1-14 | 3000 | 3000 | 500 | 305 | 103 | 5.0% |

TABLE 10
Method 2 Intermediates

| | | | Equiv. | | Maleimide | Reaction |
|---|---|---|---|---|---|---|
| | Equiv. | Equiv. | Coupling | | Valency | efficiency |
| | catalyst | linker | agent | [Maleimide] | assuming | based on |
| Int. # | (HOBt) | (MP2H) | (EDC) | (μM) | no HA loss | HA-COOH |
| 2-1 | 2000 | 1500 | 1500 | 371.2 | 111 | 5.4% |
| 2-2 | 2000 | 1500 | 1500 | 333.1 | 101 | 4.9% |
| 2-3 | 2000 | 1500 | 1500 | 371.2 | 113 | 5.5% |
| 2-4 | 2000 | 1500 | 1500 | 428.2 | 128 | 6.2% |
| 2-5 | 2000 | 1500 | 1500 | 338 | 107 | 5.2% |
| 2-6 | 3000 | 3000 | 1500 | 570.6 | 155 | 7.5% |
| 2-7 | 2000 | 3000 | 1000 | 392.5 | 121 | 5.9% |
| 2-8 | 2000 | 3000 | 1000 | 482.6 | 162 | 7.8% |

TABLE 11
Method 5 Intermediates

| | | | | Equiv. | | Maleimide | Reaction |
|---|---|---|---|---|---|---|---|
| | | Equiv. | | Coupling | | Valency | efficiency |
| | | catalyst | Equiv. | agent | [Maleimide] | assuming | based on |
| Int. # | Linker | (HOBt) | linker | (EDC) | (μM) | no HA loss | HA-COOH |
| 5-1 | MP2H | 50 | 750 | 9500 | 415.3 | 123 | 5.9% |
| 5-2 | MP2H | 50 | 1000 | 9500 | 399.64 | 138 | 6.7% |
| 5-3 | MP2H | 50 | 1000 | 9500 | 476 | 137 | 6.6% |
| 5-4* | MP2H | 50 | 500 | 9500 | 826 | 171 | 8.3% |
| 5-5* | MP2H | 59 | 593 | 11263 | 707 | 227 | 11.0% |
| 5-6 | MP2H | 50 | 1000 | 9500 | 296.43 | 95 | 4.6% |
| 5-7 | MP2H | 50 | 500 | 9500 | 234.1 | 70 | 3.4% |
| 5-8 | MP2H | 50 | 500 | 9500 | 349 | 90 | 4.4% |
| 5-9 | MP2H | 50 | 500 | 9500 | 227 | 83 | 4.0% |
| 5-10 | EMCH | 50 | 250 | 9500 | 417.9 | 122 | 5.9% |

*Maleimide concentration and valency were quantified using UV absorbance.

Alternatively, sodium hyaluronate (HA, 830 kDa) was suspended in water at 10 mg/mL or 0.1 M 2-(N-morpholino)ethanesulfonic acid (MES) buffer pH 5.7 at 4 mg/mL by gentle rotation or mixing with nutation overnight at RT. Prior to reaction, a 4 mg/mL HA stock in 0.1 M MES was made using water and ˜1 M MES pH 5.7 and mixed at RT using nutation. To 3 mg (3.6 nmol, amount will vary based on polymer composition and MW) of HA in solution is added hydroxybenzotriazole (HOBt) hydrate as a ˜5-100 mg/mL stock solution in DMSO, thiol reactive linker agent (e.g., hydrazide-X-thiol-reactive-group such as MP2H) in 1-10% DMSO (10-100 mg/mL stock), and a coupling agent (1-ethyl-3-(3-dimethylaminopropyl)carbodiimide hydrochloride (EDC)) in 0.1 M MES buffer pH 5.7. The molar equivalents for each reactant per mole of HA and per carboxylate for different methods of performing the reaction, and example methods are described in Table 12 below:

TABLE 12
Intermediate Preparation Methods

| Reactant | Method A | Method B | Method C | Method D | Method E |
|---|---|---|---|---|---|
| Coupling | 9500 | 500 | 1500 | 3000 | 6000 |
| agent | | | | | |
| (EDC) | | | | | |
| Catalyst | 50 | 3000 | 2000 | 1000 | 750 |
| (HOBt) | | | | | |
| Linker | 500-1000 | 3000 | 1500 | 1250 | 750 |
| agent | | | | | |
| (MP2H) | | | | | |

Equivalents of reactants per HA monomer (˜2000 carboxylates per 830 kDa HA).

The solution was mixed with gentle pipetting between each reagent addition and the final reaction volume was raised to 1 mL with buffer. The final mixture was allowed to react at room temperature for 45 min to 1.5 h with nutating mixer depending on Method. After the reaction, the thiol reactive biopolymer was purified using 7 kDa MWCO 5-10 mL Zeba desalting spin column equilibrated with 10% v/v glycerol (optional) pH 6.5 DPBS, loaded with crude reaction at 20% volume of resin. For NMR samples, the Zeba columns were equilibrated in and intermediate was eluted in deuterated water and not frozen. The desired intermediate was eluted into clean conical tube using centrifuge at RT, elution time ˜25-60 minutes. The intermediate was used immediately for reaction with thiol or aliquoted and flash frozen on dry ice or at −80° C. Maleimide concentration and number of modifications per polymer was determined using UV absorbance, NMR, or a modified indirect Ellman's reaction assay.

Maleimide concentrations/valencies for each reaction method are provided in Table 13 below.

TABLE 13
Maleimide Substitution Measurements
for Indirect Ellman's Method

| | Maleimide |
|---|---|
| Method | Valency* |
| A | 290 |
| B | 185 |
| C | 227 |
| D | 223 |
| E | 180 |

*The reactive maleimide concentration and approximate valency for the intermediates analyzed by NMR were determined using the Ellman's reaction assay.

NMR analysis of conjugates was performed at the Complex Carbohydrate Research Center (CCRC) at University of Georgia using at 25° C. on a Bruker Advance III spectrometer (¹H, 600.13 MHz) equipped with a 5 mm cryoprobe. After standard preparation of intermediate using Method A-E at 3-6 mL scale, the intermediate was purified into deuterated water using desalting resin and shipped to the CCRC on wet ice.

Based on analysis of the ¹H NMR spectra, all the samples contained signals corresponding to HA, MP2H, free ethyl dimethylaminopropyl urea (EDU), a byproduct of the EDC hydrolysis that was not removed during purification. The abundance of MP2H, conjugated n-acylurea adduct, and free EDU were determined relative to HA (the repeating polymer unit) by calculating the integral of the signal from characteristic peaks. Based on the integral values, the raw abundances of the HA, MP2H, free EDU, and conjugated N-acylurea (conjug. EDU) were calculated. The abundances of MP2H, conjugated N-acylurea adduct, and free EDU relative to HA, are shown in Table 14.

TABLE 14
Relative molar abundances of MP2H, free EDU, and N-acylurea
adduct relative to HA (100%) for Methods A-E

| | | Free | N-acylurea |
|---|---|---|---|
| | MP2H | EDU | adduct |
| Method A | 21.7 | 11.7 | 15.2 |
| Method B | 23.0 | 1.6 | n.d. |
| Method C | 25.9 | 6.4 | n.d |
| Method D | 26.8 | 8.0 | n.q |
| Method E | 20.7 | 18.2 | 5.0 |

n.d.: species not detected
n.q.: species present but not quantifiable due to low signal

### Example 6. Preparation of Purified Peptide-Polymer Conjugates

To obtain the purified peptide-polymer conjugates, 1.1 to 2 equivalents of peptide per maleimide was combined with the intermediates prepared by a method of Example 1 and allowed to react at either 4° C. or ambient temperature for at least 2 hours to overnight with rotation or nutating mixing (most reactions took place at RT to improve solubility). Optionally, 1 M pH 7 HEPES was added to a final concentration of 0.1 M to adjust reaction pH. In some cases, before the conjugation reaction, 10-100 equivalents of a reducing agent such as DTT or TCEP HCl was added per protein equivalent to reduce any disulfide bridging between peptides. This was removed from the peptide solution prior to conjugation by a desalting column or buffer exchange or was added to the conjugation reaction directly in the form of TCEP immobilized on polymeric beads. During the conjugation reaction, one or more of the following was added to improve the reaction efficiency: 0.5-10 mM EDTA to minimize free thiol oxidation, tween 20, carbohydrate, additional buffer, or glycerol to stabilize protein and/or help reduce non-specific interactions between protein and activated biopolymer, increased or decreased salt concentration to stabilize protein and/or help reduce non-specific interactions between protein and activated biopolymer. Unreacted peptide was removed from the peptide-polymer conjugates by one or more of the following methods: dialysis (1:100 to 1:1000) with 50-1000 kDa MWCO against an appropriate buffer (pH should be >1 unit above or below the pI of peptide) for at least two times for 4 hours each and once for at least 4 hours at 4° C.-room temperature. Tangential flow filtration against citrate buffer, DPBS pH 6-8, or 50 mM tris 150 mM NaCl pH 8-8.5 with EDTA and tween or other additives like trehalose, depending on peptide, FPLC polishing using a size exclusion column, FPLC polishing with an affinity chromatography column designed to bind the polymer component of the conjugate, or selective precipitation of the conjugates can also be used to purify conjugate away from unreacted peptide. If reaction efficiency was high enough (i.e <5% unreacted protein present), purification was not necessary.

Alternatively, to each solution of intermediate of Example 1, the peptide was added at a suitable peptide:polymer molar feed ratio and Tween-20 to a final concentration of up to 0.03% (optional). The solution was allowed to react for 2 hours to overnight while agitating by rotation (˜5 RPM) or nutation at ambient temperatures. Unreacted peptides were removed by dialysis using 50-1000 kDa MWCO membranes against each of the following buffer solutions in sequence: First, phosphate buffered saline or equivalent citrate or succinate buffered saline (pH and buffer salt used depends on peptide) with 0.010% Tween-20 (optional) for at least 4 hours, second phosphate buffered saline with 0.01% Tween-20 overnight, and phosphate buffered saline with 0.01% Tween-20 for 4 hours at 4° C. or RT, with an optional fourth dialysis step. Optionally, additives like tween 20, EDTA, and carbohydrates were added to enhance protein stability.

After the MVP purification, the conjugate was analyzed for protein concentration, protein valency, MVP radius, and binding affinity using the methods described in the stability study section. A tabular representation of MVPs synthesized using different intermediates is shown below with their resulting protein concentration, valency, dissociation constant by biolayer interferometry (BLI), and radius where determined.

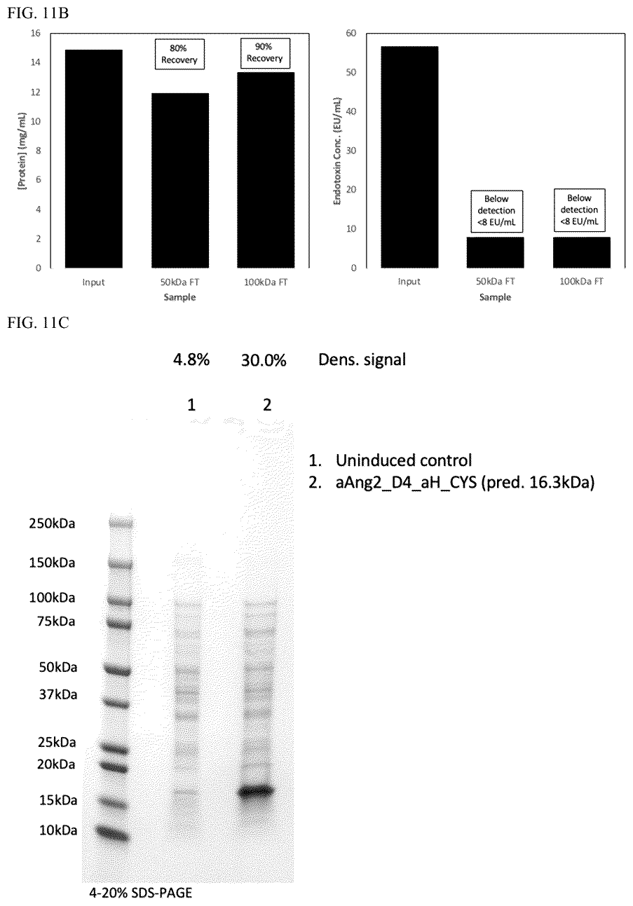

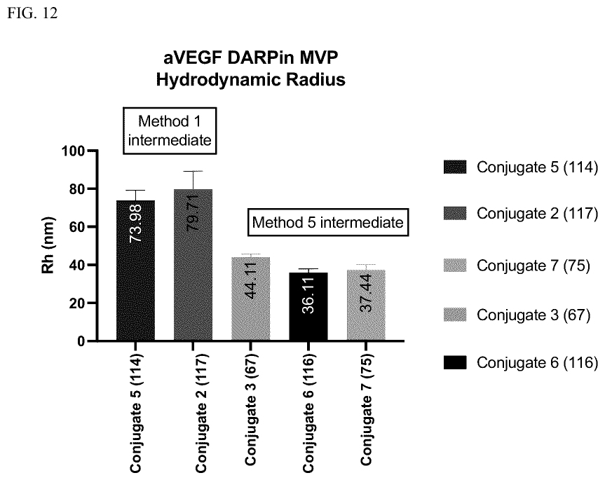

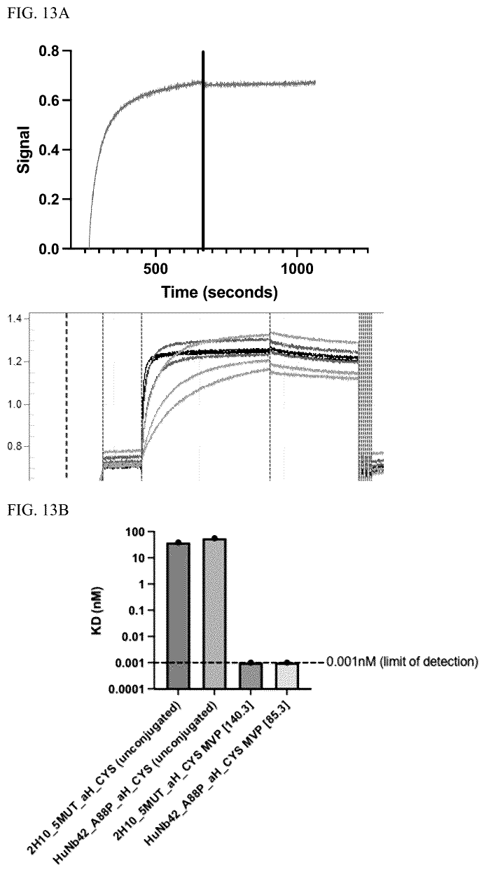

MVPs synthesized with Method 1 and Method 2 had similar or improved characteristics (final protein concentration, protein valency, radius, binding kinetics) for MVP therapeutics compared to the MVPs synthesized using Method 5 (Table 15). Examples of hydrodynamic radii comparison for DARPin MVPs synthesized with Method 1 or Method 5 intermediate are shown in FIG. 12. Examples of VEGF binding curve BLI data comparison for anti-VEGF MVPs synthesized with Method 1 or Method 5 intermediate are shown in FIG. 13.

TABLE 15
Conjugates

| | | | | Peptide | | K_{D} at | R_{g} (rz) |
|---|---|---|---|---|---|---|---|
| Conjugate | Peptide | Linker | | conc | Peptide | t = 0 | or Rₕ (nm) |
| # | SEQ ID NO: | SEQ ID NO: | Int. # | (mg/mL) | Valency | (nM)* | at t = 0 |
| 1 | 92 | — | 5-4 | 1.325 | 68 | 0.001 | 51.5 |
| 2 | 92 | — | 1-7 | 2.712 | 117 | 0.001 | 78.2 |
| 3 | 92 | — | 5-5 | 1.77 | 67 | 0.001 | 44.3 |
| 4** | 92 | — | 5-6 | 0.52 | 45 | — | — |
| 5 | 92 | — | 1-7 | 2.923 | 114 | — | 73.98 |
| 6 | 92 | — | 5-5 | 1.8 | 116 | — | 36.11 |
| 7 | 92 | — | 5-6 | 2.726 | 78 | — | 37.4 |
| 8 | 81 | 21 | 5-7 | 0.82 | 94 | 0.184 | — |
| 9 | 81 | 21 | 5-9 | 0.46 | 51 | 0.678 | — |
| 10 | 81 | 21 | 2-4 | 3.24 | 136 | 0.123 | — |
| 11 | 81 | 21 | 5-2 | 0.33 | 47 | 1.038 | — |
| 12 | 73 | 21 | 1-6 | 2.46 | 120 | — | — |
| 13 | 91 | 31 | 5-8 | 0.38 | 72 | 0.011 | — |
| 14 | 91 | 31 | 5-3 | 1.04 | 68 | 0.203 | — |
| 15 | 91 | 21 | 5-10 | 0.52 | 59 | 0.001 | — |
| 16 | 91 | 21 | 2-6 | 3.04 | 101 | 0.001 | — |
| 17 | 91 | — | 1-7 | 1.59 | 90 | 0.004 | — |
| 18 | 91 | — | 1-7 | 1.43 | 88 | 0.001 | — |
| 19 | 91 | — | 2-6 | 2.3 | 108 | 0.001 | — |
| 20 | 91 | — | 2-8 | 1.724 | 91 | 0.001 | — |
| 21 | 91 | 21 | 1-9 | 1.63 | 51 | 0.001 | — |
| 22 | 91 | 21 | 1-10 | 0.636 | 18 | 0.001 | — |
| 23 | 91 | 21 | 1-11 | 1.319 | 33 | 0.001 | — |
| 24 | 67 | 21 | 1-13 | 4.591 | 100 | — | 103 |
| 25 | 72 | 21 | 1-13 | 4.77 | 51 | 0.1 | 77 |
| 26 | 73 | 21 | 1-14 | 1.96 | 104 | | 88 |

*BLI limit of detection is 0.001 nM. Samples with this value listed in the table above were below the BLI LOD for K_{D}.
**Cy7 fluorophore labeled peptide.

Alternatively, to obtain the purified peptide-polymer conjugates, 1.1 to 2 equivalents of peptide per maleimide was combined with the HA conjugation substrates prepared by a method shown in Table 16. Conjugation reactions were allowed to react at ambient temperature for at least 2 hours to overnight with rotation or nutating mixing. 1 M pH 7 HEPES was added to a final concentration of 0.1 M to adjust reaction pH. In some cases, unreacted peptide was removed from the peptide-polymer conjugates by dialysis (1:400 to 1:1000) with 50-1000 kDa MWCO against an appropriate buffer (pH should be >1 unit above or below the pI of peptide) for at least three times for 4 hours each at 4° C.-room temperature.

TABLE 16
Reaction conditions for peptide-polymer conjugates

| | Conjugation | Conjugation | | Further | |
|---|---|---|---|---|---|
| Conjugate | Substrate | Substrate | | Peptide | Peptide |
| # | Method | (nmol) | Peptide | linker | (nmol) |
| 27 | Method A | 0.58 | Anti-VEGF | — | 141 |
| | | | VHH | | |
| | | | (SEQ ID | | |
| | | | NO: 145) | | |
| 28 | Method B | 0.60 | Anti-VEGF | — | 134 |
| | | | VHH | | |
| | | | (SEQ ID | | |
| | | | NO: 145) | | |
| 29 | Method A | 0.31 | Anti-TNFα | — | 75.5 |
| | | | VHH | | |
| | | | (SEQ ID | | |
| | | | NO: 102) | | |
| 30 | Method B | 0.32 | Anti-TNFα | — | 71.4 |
| | | | VHH | | |
| | | | (SEQ ID | | |
| | | | NO: 102) | | |
| 31 | Method B | 0.19 | Anti-VEGF | SEQ ID | 58 |
| | | | VHH | NO: 21 | |
| | | | (SEQ ID | | |
| | | | NO: 91) | | |
| 32 | Method B | 0.29 | Anti-PD1-1 | SEQ ID | 88 |
| | | | VHH | NO: 21 | |
| | | | (SEQ ID | | |
| | | | NO: 114) | | |
| 33 | Method B | 0.25 | Anti-VEGF | — | 76 |
| | | | DARPin | | |
| | | | (SEQ ID | | |
| | | | NO: 92) | | |
| 34 | Method B | 0.25 | Anti- TNFα | — | 74 |
| | | | DARPin | | |
| | | | (SEQ ID | | |
| | | | NO: 154) | | |
| 35 | Method B | 0.17 | Anti- TNFα | — | 54 |
| | | | Affibody | | |
| | | | (SEQ ID | | |
| | | | NO: 105) | | |
| 36 | Method B | 0.054 | Anti-IL- | — | 5 |
| | | | 1β scFv | | |
| | | | (SEQ ID | | |
| | | | NO: 106) | | |
| 37 | Method B | 0.054 | IL-2 | — | 18 |
| | | | (SEQ ID | | |
| | | | NO: 152 | | |
| 38 | Method B | 0.098 | IL-15 | — | 30 |
| | | | (SEQ ID | | |
| | | | NO: 153) | | |

To confirm successful conjugation, the products of the conjugation reactions were analyzed by SDS-PAGE and DLS. SDS-PAGE was used to measure the percentage of unreacted peptide that was separated by migration into the gel that was consistent with its molecular weight. After the conjugation reaction, a substantial percentage of the peptide was present as a high molecular weight conjugate at the top of the stacking gel, which was unable to migrate into the gel due to its size (>300 kDa). DLS was used to measure the hydrodynamic radii present in the reaction product. After the conjugation reaction, the highest intensity peak aligned with a Rₕ that was consistent with the conjugation substrate, indicating that the peptide was conjugated to the hyaluronic acid substrate. Data for the conjugates is shown in Table 17. Percent unreacted protein was determined by densiometric analysis of the SDS-PAGE band for the unconjugated protein that was referenced to BSA standards of known mass and then divided by the total mass loaded into each well. Hydrodynamic radius was measured using dynamic light scattering (DLS) with a Wyatt DynaPro plate reader III (25° C., 5-10 acquisitions, at 5 s, n=3 samples per conjugate). Data analysis was performed by a Jupyter notebook data analysis program to extract data of adequate quality and analyze based on highest intensity peak.

TABLE 17
Properties for each peptide-polymer conjugate

| | | | Further | Percent | |
|---|---|---|---|---|---|
| Conjugate | Conjugation | | peptide | Unconjugated | Rₕ |
| # | Substrate | Peptide | linker | Peptide | (nm) |
| 27 | Method A | Anti-VEGF | — | Aggregated | |
| | | VHH | | | |
| | | (SEQ ID | | | |
| | | NO: 145) | | | |
| 28 | Method B | Anti-VEGF | — | 24 | 85 |
| | | VHH | | | |
| | | (SEQ ID | | | |
| | | NO: 145) | | | |
| 29 | Method A | Anti-TNFα | — | Aggregated | |
| | | VHH | | | |
| | | (SEQ ID | | | |
| | | NO: 102) | | | |
| 30 | Method B | Anti-TNFα | — | 54 | 88 |
| | | VHH | | | |
| | | (SEQ ID | | | |
| | | NO: 102) | | | |
| 31 | Method B | Anti-VEGF | SEQ ID | 49 | 58 |
| | | VHH (SEQ | NO: 21 | | |
| | | ID NO: 91) | | | |
| 32 | Method B | Anti-PD1-1 | SEQ ID | — | 82 |
| | | VHH (SEQ | NO: 21 | | |
| | | ID NO: 114) | | | |
| 33 | Method B | Anti-VEGF | — | 49 | |
| | | DARPin | | | |
| | | (SEQ ID | | | |
| | | NO: 92) | | | |
| 34 | Method B | Anti- TNFα | — | 27 | 92 |
| | | DARPin | | | |
| | | (SEQ ID | | | |
| | | NO: 154) | | | |
| 35 | Method B | Anti- TNFα | — | — | 144 |
| | | Affibody | | | |
| | | (SEQ ID | | | |
| | | NO: 105) | | | |
| 36 | Method B | Anti-IL-1β | — | — | 170 |
| | | scFv | | | |
| | | (SEQ ID | | | |
| | | NO: 106) | | | |
| 37 | Method B | IL-2 | — | — | 77 |
| | | (SEQ ID | | | |
| | | NO: 152) | | | |
| 38 | Method B | IL-15 | — | — | 113 |
| | | (SEQ ID | | | |
| | | NO: 153) | | | |

Achieving high concentration of pharmaceutical formulations is often required to reach the therapeutic thresholds, maximize therapeutic durability, and/or minimize dosage volumes. However, at higher concentrations, therapeutic peptides may aggregate. For polymer-peptide conjugations, there is additional concern that interactions with the polymer substrate may contribute to aggregation or result in aggregation at lower peptide concentrations that would occur without the conjugated polymer. The HA conjugation substrates made using Method B provided polymer-peptide conjugates without measured aggregation.

To synthesize purified Conjugate #28 using Method B: 174 μL of purified conjugation intermediate from Method B was mixed with 3 μL of 2% v/v Tween20 and 26 μL of 80 mg/mL N42 anti-VEGF VHH (SEQ ID NO: 145) for 1.1 equivalents of peptide per maleimide in a 2 mL v-bottom microcentrifuge tube. Reaction pH was adjusted to pH 7 by addition of 20 μL of 1 M pH 7 HEPES for a final concentration of 0.1 M. The reaction was allowed to proceed overnight for 16 h at room temperature mixing with nutation. Unreacted peptide was removed from the peptide-polymer conjugates by dialysis (1:1000 based on initial reaction volume) with 100 kDa MWCO 200 μL microFloat-A-Lyzer dialysis cassette (Repligen) against pH 5.5 25 mM citrate, 100 mM NaCl, 0.03% tween20 at room temperature with stirring. Four total dialysis steps were performed, switching buffer three times after 4 hours each and once after 16 h overnight dialyzing.

To synthesize purified Conjugate #30 using Method B: 186 μL of purified conjugation intermediate from Method B was mixed with 3.3 μL of 2% v/v Tween20 and 14.4 μL of 80 mg/mL anti-TNFα VHH (SEQ ID NO: 102) for 1.1 equivalents of peptide per maleimide in a 2 mL v-bottom microcentrifuge tube. Reaction pH was adjusted to pH 7 by addition of 20 μL of 1 M pH 7 HEPES was added for a final concentration of 0.1 M. The reaction was allowed to proceed overnight for 16 h at room temperature mixing with nutation. Unreacted peptide was removed from the peptide-polymer conjugates by dialysis (1:1000 based on initial reaction volume) with 100 kDa MWCO 200 μL microFloat-A-Lyzer dialysis cassette (Repligen) against pH 5.5 25 mM citrate, 100 mM NaCl, 0.03% tween20 at room temperature with stirring. Four total dialysis steps were performed, switching buffer three times after 4 hours each and once after 16 h overnight dialyzing.

After the fourth dialysis step was complete, the purified conjugates were removed from the dialysis cassettes and stored at 4° C. The reaction products characterized by visual inspection, UV vis absorbance to measure purified protein conjugation, DLS to measure Rh, SDS-PAGE to determine the percent of unconjugated protein, and biolayer interferometry to measure the binding affinity as described in the Examples herein.

TABLE 18
Characterization of Conjugates #28 and #30 Prepared
from Method B Intermediate

| | | Purified Product | Percent | | | |
|---|---|---|---|---|---|---|
| | Peptide | Peptide Conc. | Unconjugated | | Rₕ | K_{d} |
| Conjugate | (SEQ ID NO:) | (mean ± COV) | Peptide | Valency | (nm) | (pM) |
| 28 | 145 | 4.956 ± 1.182% | 9 | 129 | 85 | 1.07 |
| 30 | 102 | 2.784 ± 0.772% | 3 | 137 | 88 | <1 |

### Example 7. Stability Analysis of Purified Peptide-Polymer Conjugates

MVP stability was assessed by setting up a long term accelerated in vivo stability by maintaining the MVPs at 37° C. at 5-10X the therapeutic concentration in pH 7.3 vitreous mimetic buffer (Table 19) or pH 7.4 PBS 0.01% tween 20. MVP stability was assessed using SEC MALS or SEC, DLS, and/or BLI analysis of samples removed after various times.

TABLE 19
Vitreous Mimetic Buffer Composition

| Component | mg/mL |
|---|---|
| NaCl | 7.14 |
| KCl | 0.38 |
| CaCl₂ 2H₂O | 0.154 |
| MgCl₂ 6H₂O | 0.2 |
| dibasic NaPhosphate (NaH₂PO₄) | 0.42 |
| NaHCO₃ | 2.1 |
| Dextrose | 0.92 |
| lactic acid | 0.358 |
| CuSO₄ | 8.28 × 10⁻⁵ |
| ZnSO₄ heptahydrate | 0.000561 |
| FeCl₂ tetrahydrate | 0.000618 |
| Transferrin (2 Fe binding sites) | 0.0878 |
| Reduced Glutathione (GSH) | 0.0154 |

Long-term 37° C. stability studies were set up to assess composition impact on MVP stability. MVPs were synthesized under sterile conditions and diluted to around 0.4 mg/mL in a sterile filtered human vitreous mimetic buffer. This concentration is 5X higher than intravitreal therapeutic concentration of our predicted clinical dose. The samples were either filtered using sterile 0.2 or 5 μm spin filters before use or mixed with 0.01% sodium azide as an anti-microbial agent. Then, several 100 μL aliquots of each sample were added to wells of a sterile 96 well plate with one day 0 aliquot reserved at 4° C. The remaining wells were filled with a sterile filtered human vitreous buffer+0.01% sodium azide to minimize evaporation. The plate was incubated in a standard tissue culture incubator at 37° C. with 5% CO₂. At discrete timepoints, one aliquot from each sample was removed from the plate under sterile conditions and analyzed. First, the UV-VIS spectrum of the sample was taken from 200-600 nm in 10 nm steps to monitor any dramatic changes in sample composition. Then, the protein concentration is measured to adjust for any differences in volume that may have occurred. The binding affinity to is measured using BLI. The change in Kₒₙ (association constant) or K_{D} over time is used to assess relative stability. To monitor changes in radius over time, the samples are spun for 5 minutes at 5000 g to remove any large aggregates or dust particles and the Rh is measured using DLS without any sample dilution.

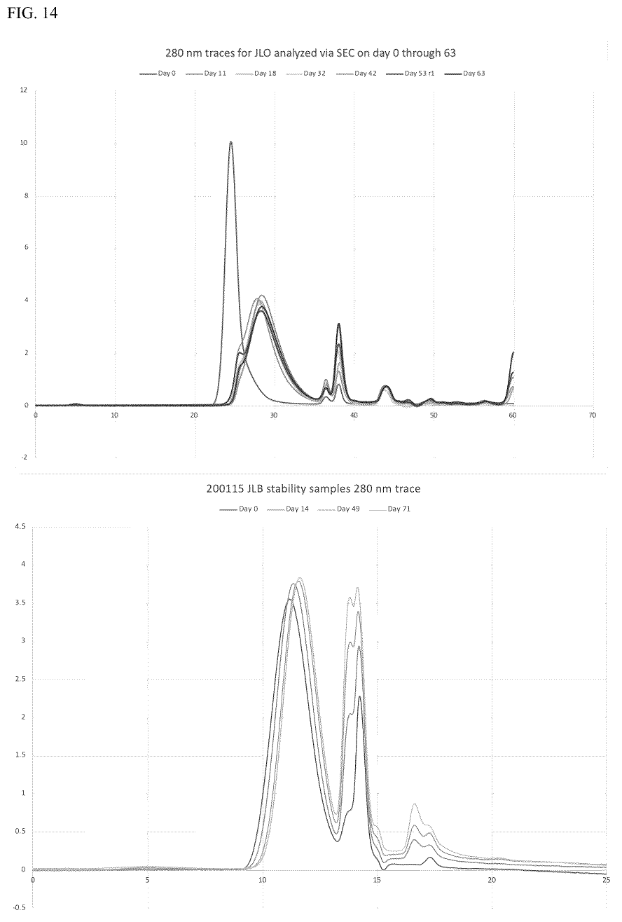

Stability study samples were analyzed using HPLC size exclusion chromatography (SEC). This method was also used to analyze MVP formation and percent unreacted protein after purification. To assess stability via SEC, MVP was filtered to remove particles and analyzed using a Shodex 1 MDa Ohpak LB-804, Shodex KW-404 or 405, or Phenomenex PolySep6000 column with DPBS or appropriate solvent as the mobile phase to get baseline trace at 280 nm, 230 nm, etc. After various time points samples were removed and analyzed using the same SEC method. Increases in retention time and peak width relative to the baseline sample indicated degradation. In addition, decreases in MVP peak area and/or increases in monomer and dimer protein species peak area also indicate MVP degradation. Percent conjugate loss was quantified by comparing peak area differences with time. In the future, the SEC stability analysis will be coupled with MALS to quantify molecular weight and valency changes of the conjugate with age at different temperatures. Representative SEC data for Method 5 or Method 1 intermediate DARPin MVP samples aged at 37° C. for up to 71 days is shown in FIG. 14. Due to its larger size, Conjugate 2 could not be analyzed on the same column used to analyze Conjugate 1 in this data.

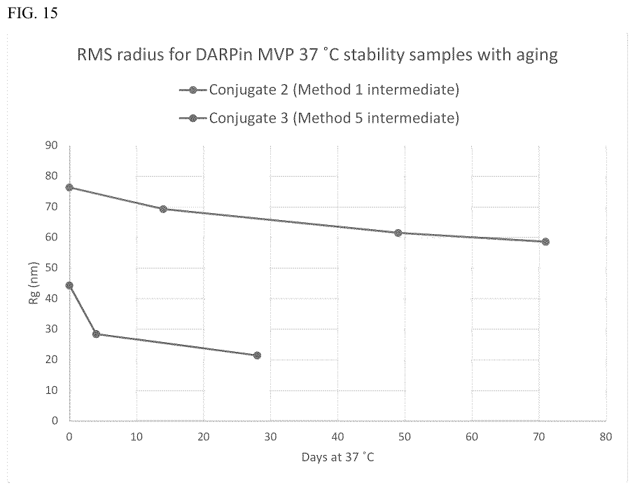

Stability study samples were also analyzed by coupling SEC with MALS analysis for determination of MVP radius of gyration (R_{g,z}) and molecular weight at different time points after aging at 37° C. For this, the conjugate stability samples were loaded into a glass vial insert (250 μL capacity) nested in a 2 mL HPLC vial and capped. For HPLC analysis, 5-20 μg of MVP (based on protein) was injected on a 1260 Infinity Agilent HPLC system with isocratic pump, autosampler, thermostatted column compartment, and variable wavelength detector set to monitor at 280 nm (or equivalent instrument), using a Shodex KW-405-4F (4.6×300 mm, 0.35 mL/min flow rate) or LB-804 or 806 (8×300 mm, 0.4 mL/min flow rate, for analyzing unconjugated VHH peaks) with their respective guard column. For analysis, column compartment was held at 30° C. using an isocratic method with 0.1 μm filtered pH 7.4 DPBS, 200 mM KCl, 100 mM urea, 50 mM sodium phosphate pH 6 with 0.025% sodium azide, or 0.1 μm filtered 300 mM NaCl 10 mM sodium phosphate, 0.025% SDS, 0.025% sodium azide pH 6.0 mobile phase made with HPLC grade water, allowing at least 2 column volumes of mobile phase to elute after sample injection, or a 60 min run time total. A Dawn Heleos II MALS instrument and Optilab T-rEX differential refractive index detectors (Wyatt Technology) or equivalent instrumentation were in line with the HPLC, downstream of the UV detector. MALS and dRI detector parameters for protein-polymer conjugate analysis using Astra software (Wyatt Technology) are listed in Table 20. System-specific calibration numbers, normalization coefficients, delay volumes, and band broadening terms were determined for the system prior to analysis. Representative SEC traces for MVP stability samples for the EDC range are shown in FIG. 14. Increases in retention time indicated loss in size/shrinking/degradation of the MVP with aging. In FIG. 14, the high EDC conjugate (Conjugate 1) demonstrated a much larger increase in retention time and contraction of radius compared to the low EDC MVP (conjugate 2). Peak broadening also suggested an increase in polydispersity of the sample with aging possibly indicating sample decomposition. The radius loss was further verified by MALS for samples in accelerated aging studies shown in FIG. 15.

TABLE 20
MALS and dRI instrument parameters for SEC MALS analysis

| Solvent | | |
|---|---|---|
| Name | PBS, Aqueous | |
| Temp | 25° | C. |
| RI calculated at 664 nm | 1.3308 | |
| Model | Cauchy | |
| Viscosity | 0.8902 | cP |
| Model | Kestin | |
| Thermal expansion | 2.58E−04 | |
| Model | Polynomial | |
| Sample | | |
| Protein dn/dc | 0.185 | |
| Polymer dn/dc* | 0.159 | |
| Protein extinction | 2.171 | mL/(mg*cm) |
| coefficient* | | |
| Polymer extinction | 0.022 | mL/(mg*cm) |
| coefficient* | | |
| MALS | | |
| sample cell | Fused silica | |
| Collection interval | 0.5 | s |
| wavelength | 664 | nm |
| Temperature | 25° | C. |
| dRI | | |
| wavelength | 658 | nm |
| Temperature | 25° | C. |

| | |
|---|---|
| LS analysis | Protein-polymer |
| | conjugate |
| Model | Debye |
| Fit Degree | 2 |
| Fit R squared | >0.99 |

Stability was also assessed based on the change in macromolecular size (e.g., Rh) using DLS. For stability analysis based on radius change with aging at 37° C. using DLS, samples are removed from the 37° C. stability study conditions for analysis at various time points. All samples and buffers are room temperature. The solution is diluted in sterile 0.1 um filtered formulation buffer without polysorbate 20 to a final concentration of 100 nM in 100 μL (typically a 1:10 dilution) and mixed by gentle trituration in a 1.5 mL centrifuge tube. Large aggregates and dust particles could be removed by spinning the tubes at 5000 g for 5 minutes in a centrifuge. For single cuvette measurements in a NanoStar, a 40 μL sample of the sample solution was loaded into a Wyatt Technology disposable microcuvette (Wyatt Cat #WNDMC) with cap, tapped to remove bubbles, and placed into the instrument for analysis. For multiple readings using the plate reader, 25-35 μL of sample was added to a clear bottom black well 384 well plate (Corning Cat #P8802-384 or similar). Bubbles in the samples were removed by spinning briefly in a centrifuge with a plate adaptor and then removed with either a pipette tip or by gently blowing with 70% EtOH vapor from a squirt bottle. Instrument settings for this and the other sample analyses by DLS in this document are presented in Table 21. Any peaks greater than 1000 nm should have a <6% Intensity. DLS acquisition parameters are shown in Table 21 below.

TABLE 21
DLS Acquisition Parameters

| Instrument | | |
|---|---|---|
| Laser wavelength | 664 | nm |
| Laser power | 100% | |
| Auto attenuation | On | |
| Temp | 25° | C. |
| Acquisition time (s) | 5-10 | |
| Acquisition # | 5 | |
| Fixed Parameters | | |
| Correlation function low cutoff | 1.5 | μs |
| Correlation function high cutoff (μs) | 103000 | μs |
| Peak radius low cutoff | 0 | nm |
| Peak radius high cutoff | 1 × 10⁶ | nm |

| | |
|---|---|
| Analysis type | Dynals |
| Measurement time limit factor | 5 |
| Auto-attenuation time limit | 60 |
| Sample | |
| Mw-R model | Globular proteins |
| Solvent | PBS |
| dn/dc | 0.185 |
| Rg model | Sphere |

Table 22 below shows the MVP radius changes with accelerated 37° C. aging for MVP samples synthesized using different methods. The hydrodynamic radius or radius of gyration was determined by DLS or MALS at t=0 and at various timepoints after aging at 37° C. In these examples, MVPs synthesized with low EDC (Method 1) had improved 37° C. stability based a smaller contraction/change in radius with aging. This suggested that the presence of N-acylurea adducts destabilized the conjugate prepared with methods using higher amounts of EDC.

TABLE 22
MVP Radius over Time

| | R_{g} (rz) | | | | | | |
|---|---|---|---|---|---|---|---|
| | or Rₕ (nm) | | | | | | |
| | prior to | Days Aging | R_{g} (rz) | | Days Aging | R_{g} (rz) | |
| Conjugate | stability | at 37° C., | or Rₕ (nm) | Radius | at 37° C., | or Rₕ (nm) | Radius |
| # | study | sample 1 | sample 1 | change | sample 2 | sample 2 | change |
| 1 | 51.5 | 2 | 34.5 | −33% | | | |
| 2 | 78.2 | 14 | 71.4 | −9% | 71 | 61.4 | −21% |
| 3 | 44.3 | 4 | 28.4 | −36% | 28 | 21.4 | −52% |

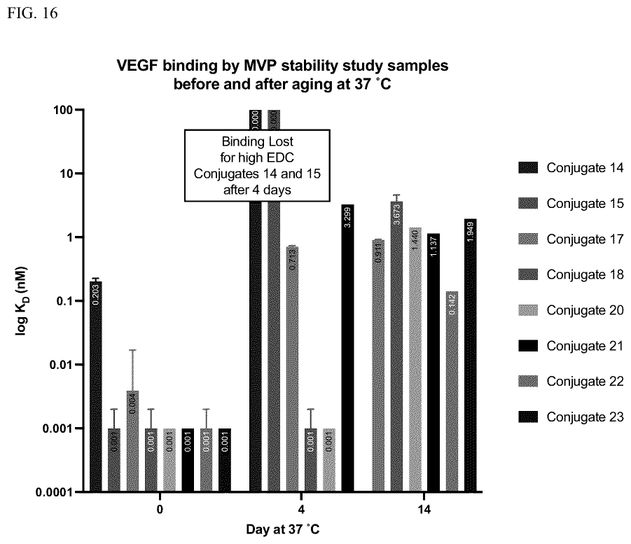

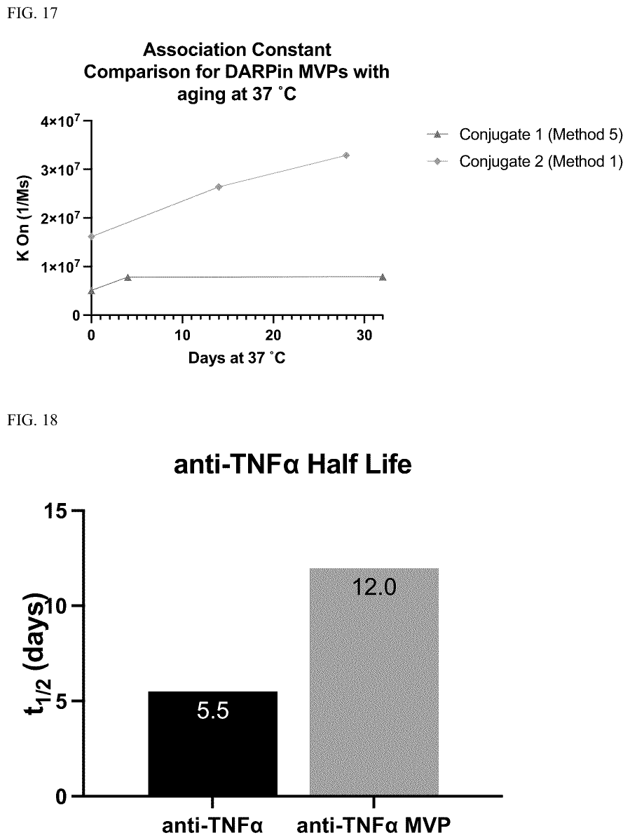

The change in binding affinity over time using biolayer interferometry (BLI) was also determined. To perform BLI experiments, samples were removed from the 37° C. stability study conditions for analysis at various time points. All reagents were equilibrated to room temperature before use for at least 30 minutes. Two probes per sample were equilibrated (one for kinetic assay and one for ligand free control) in 250 μL BLI buffer (PBS pH 7.4, 0.2% Tween and 0.2% BSA filtered at 0.2 μm) for at minimum 10 min in a Gator Bio Max plate. Ligands were diluted to a fixed concentration of 25-100 nM based on performance in pilot reactions in BLI buffer. Analytes were prepared at the top concentration determined in pilot reactions in BLI buffer and serially diluted 1:3 two to five more times using BLI buffer. Black flat-bottom non-coated 96 well plates (Greiner Bio One Cat #655209 or similar) were loaded column-wise with 200 μL of ligand, analyte dilutions and one column of BLI buffer for each column of ligand and analyte. One well in each column of analyte should be BLI buffer to be used as a blank for reference subtraction. No bubbles were present in the wells and removed with either a pipet tip or by gently blowing with 70% EtOH vapor from a squirt bottle. The plate was placed in the Gator on a tilted platform set to 25° C. Gator K assay loading and kinetic steps were set up using double reference and step times shown in Table 24. Ligand was loaded until signal reaches between 0.4 and 0.6 nm then return to buffer column for a baseline measurement for 60 s. The kinetic reads were started using the step parameters in Table 24. When kinetic reads were complete with ligand-loaded probes, a ligand free control was run using new probes that were not loaded with the ligand. The same kinetic assay timing and same sample wells that were analyzed with ligand loaded probes were used. This data was used to correct for any non-specific interactions between the sample and probe. Representative BLI data for high (Method 5) and low (Method 1) EDC MVP samples before and after accelerated aging at 37° C. are shown in FIG. 16 (K_{D}) and FIG. 17 (Kₒₙ).

When kinetic assay was complete, data was analyzed in the results and analysis section of the Gator software. The raw data was corrected to include the association time after 1 second to 180 seconds. Y axis was aligned to the beginning of the association step and turn on interstep correction. Savitzky-Goaly Filtering of data was used. The samples were set for a double reference by denoting which probes and wells are buffer references in the software. Then, the reference subtraction formula was edited for each assay so that for each assay it was a double reference with the equation of (Kinetic Assay well-Ligand Free Assay well)-(Kinetic Assay buffer reference well—Ligand free assay buffer well). All titrations of the same MVP were grouped by color and the parameters adjusted to a 1:1 binding model that included both association and dissociation with global, Rmax unlinked fitting. The window of interest was moved to include only 100 seconds of dissociation. The binding curve was fitted and checked that the residuals did not vary from the actual curve more than 10%, that the full R² was >0.98 and the Full X² was <3.0. The kinetics were calculated, and the K_{D}, Kₒₙ and response were noted. When different samples had the same K_{D} result, the association constant Kₒₙ was used to differentiation binding affinity between the different constructs (i.e. FIG. 17), where a higher association constant indicated faster binding kinetics.

TABLE 23
BLI Ligands and analyte pairings

| | | | | Catalog |
|---|---|---|---|---|
| BLI Ligand | Tags | Analyte | Supplier | Number |
| Human | Avi, His | anti- | Acro | TNA-H82E3 |
| TNFα | | TNFα | Biosystems | |
| Human | Avi | anti- | R&D | AVI-293-050 |
| VEGF 165 | | VEGF | Systems | |

TABLE 24
BLI method parameters and results specifications
for kinetic quantitation

| Parameter | Wells Used | Step time (s) or info |
|---|---|---|
| Probe equilibration | Buffer in Max Plate | >600 |
| Basic Parameters | | 5 Hz, 30 s equilibration, |
| | | 1000 rpm shaking |
| Buffer | | PBS pH 7, 0.2% |
| | | Tween and 0.2% |
| | | BSA filtered at 0.2 μm |
| BLI Experiment | | |
| Parameters | | |
| Baseline | Buffer Column 1 | 60 |
| Ligand loading | 100-25 nM Ligand | When loading signal is |
| | | at 0.4-0.6 |
| Baseline | Buffer Column 1 | 90 |
| Association | MVP Sample(s) | 180 |
| Dissociation | Buffer Column 2 | 300 |
| Ligand free control | | |
| (with blank probes) | | |
| Baseline | Buffer Column 2 | 90 |
| Association | MVP Sample(s) | 180 |
| Dissociation | Buffer Column 2 | 300 |

A tabular representation of the binding kinetics upon accelerated 37° C. aging for MVP samples synthesized using different methods is shown below. The dissociation constant for samples was determined by BLI at various timepoints after aging at 37 TC. In these examples, MVPs synthesized with Method 1 and Method 2 had similar or improved 37° C. stability based on therapeutic target binding capacity and a similar or smaller change in dissociation constant with aging. The anti-VEGF VHH peptide MVPs synthesized with intermediate Method 5 lost all binding ability after brief time aging while the examples synthesized with Method 1 or 2 demonstrated target binding capacity throughout the study, suggesting the presence of N-acylurea adducts destabilized the therapeutic.

TABLE 25
Binding of Conjugates over Time

| | K_{D} at | Days Aging | K_{D} at | | Days Aging | K_{D} at Aging | Change in |
|---|---|---|---|---|---|---|---|
| Conjugate | t = 0 | at 37° C., | Aging | Change in | at 37° C., | Day (nM), | K_{D} to t = 0, |
| # | (nM) | sample 1 | Day (nM) | K_{D} to t = 0 | sample 2 | Sample 2 | Sample 2 |
| 1 | 0.001 | 11 | 0.001 | 1 | 32 | 0.075 | 75 |
| 2 | 0.001 | 14 | 0.001 | 1 | 28 | 0.017 | 17 |
| 8 | 0.184 | 3 | 0.557 | 3 | 5 | No binding | Binding lost |
| | | | | | | observed | |
| 11 | 1.038 | 3 | 6.755 | 7 | 5 | 21.89 | 21 |
| 14 | 0.203 | 4 | No binding | Binding lost | — | — | — |
| | | | observed | | | | |
| 15 | 0.001 | 4 | No binding | Binding lost | — | — | — |
| | | | observed | | | | |
| 16 | 0.001 | 4 | 0.654 | 654 | 11 | 0.648 | 648 |
| 17 | 0.004 | 4 | 0.713 | 178 | 14 | 0.911 | 228 |
| 18 | 0.001 | 4 | 0.001 | 1 | 14 | 3.67 | 3670 |
| 19 | 0.001 | 4 | 0.185 | 185 | — | — | — |
| 20 | 0.001 | 4 | 0.001 | 1 | 14 | 1.44 | 1440 |
| 21 | 0.001 | 4 | 3.299 | 3299 | 14 | 1.137 | 1137 |
| 22 | 0.001 | 4 | 5.87 | 5870 | 14 | 0.1419 | 142 |
| 23 | 0.001 | 4 | 6.964 | 6964 | 14 | 1.949 | 1949 |

### Example 8. In Vivo Half Life of a Purified Peptide-Polymer Conjugate

An extended intravitreal retention time of the conjugates was shown in a well-established pharmacokinetics model. New Zealand White rabbits (n=9) were divided into 3 groups randomized by weight. All animals received a 50-μL ITV injections of hu_anti-TNFα_aH MVP (SEQ ID NO:102)+HyA (850 kDa) (“anti-TNFα MVP”) in the left eye and the unconjugated VHH (SEQ ID NO: 102) (“anti-TNFα”) in the right eye using a 31 G insulin syringe. Both eyes received an equivalent molar dose of antibody. At 1 hour, 5 days and 10 or days post injection, one group of three rabbits are sacrificed, and their eyes enucleated for analysis of intravitreal VHH. Both eyes were flash frozen, and the vitreous, retina, and aqueous humor were isolated from the frozen eye. Each tissue sample was then homogenized with a bead beater. After homogenization, the VHH concentrations were quantified either using ELISA or by digesting the peptide using trypsin and subjecting the samples to LC/mass spectrometry, or a similar method. Representative results for the extended intravitreal half-life in rabbit eyes after bioconjugation are shown in FIG. 18.

The method for fluorescence tagging of the peptide for this study is as follows. Mouse tumor models for evaluating the clearance rate of proteins from solid tumors were used to measure the intratumoral (IT) half-life of MVPs to maximize the parameters for tumor retention. We used antibodies tagged using the amine-reactive Sulfo-Cy7 NHS ester (Broadpharm Cat #BP-22541) or Alexa Fluor 750 near-infrared fluorophore by the following method, which was conducted under aseptic conditions. First, the dye was dissolved DMSO at 10 mg/mL concentration. Then, the protein at 5.0-10.0 mg/mL concentration was mixed with 0.1 M sodium bicarbonate at a 3:2 vol:vol ratio. Lastly, the fluorophore was added at a 1:2 protein:fluorphore molar ratio, mixed well, and incubated at room temperature for one hour on a nutator protected from the light by covering with foil. The NHS esters were quenched by adding 1.5 M Tris buffer pH 8.5 at 10% of the reaction volume and mixed on a nutator for another 10 minutes. The tagged protein was purified away from the unreacted fluorophore using a NAP-10 desalting column (illustra Cat #17-0854-01) that was equilibrated with PBS pH 7.0+0.01% Tween-20 according to the manufacturer's directions. The protein concentration and degree of Cy7 labeling was determined by the absorbance at 280 and 750 nm. The protein as stored on ice and within 3 hours of Sulfo-Cy7 labeling, was used for MVP synthesis following the protocols described above. In each case, the final product was then sterile filtered, and stored at 4° C. protected from light until it was used in the animal studies.

Although the foregoing invention has been described in some detail by way of illustration and Example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference. Where a conflict exists between the instant application and a reference provided herein, the instant application shall dominate.

TABLE 26
Sequences

| Name | Sequence |
|---|---|
| framework | QVQLVESGGGLVQPGGSLRLSCAASG (SEQ ID NO: 5) |
| region 1 | |
| framework | MGWFRQAPGKEREFVAAI (SEQ ID NO: 6) |
| region 2 | |
| framework | YADSVKGRFTISRDNSKNTVYLQMNSLRPEDTAVYYCAA (SEQ ID NO: 7) |
| region 3 | |
| framework | YWGQGTLVTVSS (SEQ ID NO: 8) |
| region 4 | |
| Nb42 | FAYSTYS (SEQ ID NO: 9) |
| CDR1 | |
| Nb42 | NSGTFRLW (SEQ ID NO: 10) |
| CDR2 | |
| Nb42 | RAWSPYSSTVDAGDFR (SEQ ID NO: 11) |
| CDR3 | |
| 2H10 | RRFSIEA (SEQ ID NO: 12) |
| CDR1 | |
| 2H10 | DSGGSTD (SEQ ID NO: 13) |
| CDR2 | |
| 2H10 | IGSSWYGRGLD (SEQ ID NO: 14) |
| CDR3 | |
| aTNFa-mu | GTFSSII (SEQ ID NO: 15) |
| CDR1 | |
| aTNFa-mu | SWSGGTTV (SEQ ID NO: 16) |
| CDR2 | |
| aTNFa-mu | RPYQKYNWASASYNV (SEQ ID NO: 17) |
| CDR3 | |
| E1-1 CDR1 | GGSDAGT (SEQ ID NO: 18) |
| E1-1 CDR2 | SWAGTAWR (SEQ ID NO: 19) |
| E1-1 CDR3 | LGSYEMDHH (SEQ ID NO: 20) |
| aH linker | AEAAAKEAAAKEAAAKAGC (SEQ ID NO: 21) |
| AE3K2R(2) | AEEEKRKAEEEKRKAEEEAGC (SEQ ID NO: 22) |
| linker | |
| AE3K2R(3) | AEEEKRKAEEEKRKAEEEKRKAEEEAGC (SEQ ID NO: 23) |
| linker | |
| E4K4(2) | AEEEEKKKKEEEEKKKKAGC (SEQ ID NO: 24) |
| linker | |
| EA3K(2) | AEAAAKEAAAKAGC (SEQ ID NO: 25) |
| linker | |
| Alfa linker | PSRLEEELRRRLTEGC (SEQ ID NO: 26) |
| MyosinVI | AEEEEKKKQQEEEAERLRRIQEEMEKERKRREEDEERRRKEEEERRMK |
| linker | LEMEAKRKQEEEERKKREDDEKRKKKAGC (SEQ ID NO: 27) |
| Spot linker | PDRVRAVSHWSSC (SEQ ID NO: 28) |
| GT9 linker | GTGTGTGTGTGTGTGTGTGC (SEQ ID NO: 29) |
| Modified | TPTTPPTPTPGTPPGGC (SEQ ID NO: 30) |
| Rigid linker | |
| Rigid | SPSTPPTPSPSTPPGGC (SEQ ID NO: 31) |
| blank | SEQ ID NO: 32-50 |
| 2H10 | QVQLVESGGGLVQAGGSLRLSCLASGRRFSIEAMGWYRQAPGKQREL |
| | VAIIDSGGSTDYVDSAKGRFTISRDNRKNTVDLQMNSLKDEDTGVYYC |
| | NVIGSSWYGRGLDYWGNGTQVTVSS (SEQ ID NO: 51) |
| 2H10 | QVQLVESGGGLVQAGGSLRLSCLASGRRFSIEAMGWYRQAPGKQREL |
| N112Q | VAIIDSGGSTDYVDSAKGRFTISRDNRKNTVDLQMNSLKDEDTGVYYC |
| | NVIGSSWYGRGLDYWGQGTQVTVSS (SEQ ID NO: 52) |
| 2H10 | QVQLVESGGGLVQAGGSLRLSCLASGRRFSIEAMGWYRQAPGKQREL |
| 5MUT | VAIIDSGGSTDYVDSAKGRFTISRDNAKNTVYLQMNSLKPEDTAVYYC |
| | NVIGSSWYGRGLDYWGQGTQVTVSS (SEQ ID NO: 53) |
| 2H10 | QVQLVESGGGLVQAGGSLRLSCLASGRRFSIEAMGWYRQAPGKQREL |
| 6MUT | VAIIDSGGSTDYADSAKGRFTISRDNAKNTVYLQMNSLKPEDTAVYYC |
| | NVIGSSWYGRGLDYWGQGTQVTVSS (SEQ ID NO: 54) |
| Hu2H10 | QVQLVESGGGLVQPGGSLRLSCLASGRRFSIEAMGWYRQAPGKQRELV |
| 5MUT | AIIDSGGSTDYVDSAKGRFTISRDNSKNTVYLQMNSLRAEDTAVYYCN |
| | VIGSSWYGRGLDYWGQGTLVTVSS (SEQ ID NO: 55) |
| Hu2H10 | QVQLVESGGGLVQPGGSLRLSCLASGRRFSIEAMGWYRQAPGKQRELV |
| 5MUT | AIIDSGGSTDYVDSAKGRFTISRDNSKNTVYLQMNSLKPEDTAVYYCN |
| R86K | VIGSSWYGRGLDYWGQGTLVTVSS (SEQ ID NO: 56) |
| A87P | |
| Hu2H10 | QVQLVESGGGLVQPGGSLRLSCLASGRRFSIEAMGWYRQAPGKQRELV |
| 5MUT | AIIDSGGSTDYVDSAKGRFTISRDNSKNTVYLQMNSLRAEDTAVYYCN |
| L115Q | VIGSSWYGRGLDYWGQGTQVTVSS (SEQ ID NO: 57) |
| Hu2H10 | QVQLVESGGGLVQPGGSLRLSCLASGRRFSIEAMGWYRQAPGKQRELV |
| 5MUT | AIIDSGGSTDYVDSAKGRFTISRDNSKNTVYLQMNSLKPEDTAVYYCN |
| R86K | VIGSSWYGRGLDYWGQGTQVTVSS (SEQ ID NO: 58) |
| A87P | |
| L115Q | |
| blank | SEQ ID NO: 59-60 |
| Nb42 | QVQLQESGGGSLQAGASLRLSCAASGFAYSTYSMGWFRQVSGKEREG |
| | VATINSGTFRLWYTDSVKGSFTISRDNAKNMLYLQMNSLKPEDTAIYY |
| | CAARAWSPYSSTVDAGDFRYWGQGTQVTVSS (SEQ ID NO: 61) |
| HuNb42 | QVQLVESGGGLVQPGGSLRLSCAASGFAYSTYSMGWFRQAPGKEREA |
| | VATINSGTFRLWYTDSVKGRFTISRDNSKNTLYLQMNSLRAEDTAVYY |
| | CAARAWSPYSSTVDAGDFRYWGQGTLVTVSS (SEQ ID NO: 62) |
| HuNb42 | QVQLVESGGGLVQAGGSLRLSCAASGFAYSTYSMGWFRQAPGKEREA |
| P14A | VATINSGTFRLWYTDSVKGRFTISRDNSKNTLYLQMNSLRAEDTAVYY |
| | CAARAWSPYSSTVDAGDFRYWGQGTLVTVSS (SEQ ID NO: 63) |
| HuNb42 | QVQLVESGGGLVQPGGSLRLSCAASGFAYSTYSMGWFRQAPGKEREA |
| T61A | VATINSGTFRLWYADSVKGRFTISRDNSKNTLYLQMNSLRAEDTAVYY |
| | CAARAWSPYSSTVDAGDFRYWGQGTLVTVSS (SEQ ID NO: 64) |
| HuNb42 | QVQLVESGGGLVQPGGSLRLSCAASGFAYSTYSMGWFRQAPGKEREA |
| S75A | VATINSGTFRLWYTDSVKGRFTISRDNAKNTLYLQMNSLRAEDTAVYY |
| | CAARAWSPYSSTVDAGDFRYWGQGTLVTVSS (SEQ ID NO: 65) |
| HuNb42 | QVQLVESGGGLVQPGGSLRLSCAASGFAYSTYSMGWFRQAPGKEREA |
| L79V | VATINSGTFRLWYTDSVKGRFTISRDNSKNTVYLQMNSLRAEDTAVYY |
| | CAARAWSPYSSTVDAGDFRYWGQGTLVTVSS (SEQ ID NO: 66) |
| HuNb42 | QVQLVESGGGLVQPGGSLRLSCAASGFAYSTYSMGWFRQAPGKEREA |
| A88P | VATINSGTFRLWYTDSVKGRFTISRDNSKNTLYLQMNSLRPEDTAVYY |
| | CAARAWSPYSSTVDAGDFRYWGQGTLVTVSS (SEQ ID NO: 67) |
| HuNb42 | QVQLVESGGGLVQPGGSLRLSCAASGFAYSTYSMGWFRQAPGKEREA |
| L121Q | VATINSGTFRLWYTDSVKGRFTISRDNSKNTLYLQMNSLRAEDTAVYY |
| | CAARAWSPYSSTVDAGDFRYWGQGTQVTVSS (SEQ ID NO: 68) |
| HuNb42 | QVQLVESGGGLVQPGGSLRLSCAASGFAYSTYSMGWFRQAPGKEREA |
| T61A A88P | VATINSGTFRLWYADSVKGRFTISRDNSKNTLYLQMNSLRPEDTAVYY |
| | CAARAWSPYSSTVDAGDFRYWGQGTLVTVSS (SEQ ID NO: 69) |
| HuNb42 | QVQLVESGGGLVQPGGSLRLSCAASGFAYSTYSMGWFRQAPGKEREA |
| A88P | VATINSGTFRLWYTDSVKGRFTISRDNSKNTLYLQMNSLRPEDTAVYY |
| L115Q | CAARAWSPYSSTVDAGDFRYWGQGTQVTVSS (SEQ ID NO: 70) |
| aTNF-a mu | QVQLQDSGGGLVQAGGSLRLSCAASGGTFSSIIMAWFRQAPGKEREFV |
| | GAVSWSGGTTVYADSVLGRFEISRDSARKSVYLQMNSLKPEDTAVYYC |
| | AARPYQKYNWASASYNVWGQGTQVTVSS (SEQ ID NO: 71) |
| aTNF-a mu | QVQLQESGGGLVQAGGSLRLSCAASGGTFSSIIMAWFRQAPGKEREFV |
| 3MUT | GAVSWSGGTTVYADSVKGRFTISRDSARKSVYLQMNSLKPEDTAVYY |
| | CAARPYQKYNWASASYNVWGQGTQVTVSS (SEQ ID NO: 72) |
| aTNF-a | QVQLQESGGGLVQPGGSLRLSCAASGRTFSDHSGYTYTIGWFRQAPGK |
| VHH | EREFVARIYWSSGNTYYADSVKGRFAISRDIAKNTVDLTMNNLEPEDT |
| | AVYYCAARDGIPTSRSVESYNYWGQGTQVTVSS (SEQ ID NO: 73) |
| blank | SEQ ID NO: 74-80 |
| E1-1 | EVQLQASGGGFVQPGGSLRLSCAASGGGSDAGTMGWFRQAPGKEREF |
| | VSAISWAGTAWRYYADSVKGRFTISRDNSKNTVYLQMNSLRAEDTAT |
| | YYCALGSYEMDHHYWGQGTQVTVSS (SEQ ID NO: 81) |
| E1-1 F11L | EVQLQASGGGLVQPGGSLRLSCAASGGGSDAGTMGWFRQAPGKEREF |
| | VSAISWAGTAWRYYADSVKGRFTISRDNSKNTVYLQMNSLRAEDTAT |
| | YYCALGSYEMDHHYWGQGTQVTVSS (SEQ ID NO: 82) |
| E1-1 S49A | EVQLQASGGGFVQPGGSLRLSCAASGGGSDAGTMGWFRQAPGKEREF |
| | VAAISWAGTAWRYYADSVKGRFTISRDNSKNTVYLQMNSLRAEDTAT |
| | YYCALGSYEMDHHYWGQGTQVTVSS (SEQ ID NO: 83) |
| E1-1 F11L | EVQLQASGGGLVQPGGSLRLSCAASGGGSDAGTMGWFRQAPGKEREF |
| S49A | VAAISWAGTAWRYYADSVKGRFTISRDNSKNTVYLQMNSLRAEDTAT |
| | YYCALGSYEMDHHYWGQGTQVTVSS (SEQ ID NO: 84) |
| E1-1 CDR | EVQLQASGGGFVQPGGSLRLSCAASGRRFSIEAMGWFRQAPGKEREFV |
| | SAIDSGGSTDYADSVKGRFTISRDNSKNTVYLQMNSLRAEDTATYYCA |
| | VIGSSWYGRGLDYWGQGTQVTVSS (SEQ ID NO: 85) |
| blank | SEQ ID NO: 86-90 |
| anti-VEGF | DVQLVESGGGLVQPGGSLRLSCAASGRTFSSYSMGWFRQAPGKEREFV |
| VHH | VAISKGGYKYDAVSLEGRFTISRDNAKNTVYLQINSLRPEDTAVYYCAS |
| | SRAYGSSRLRLADTYEYWGQGTLVTVSS (SEQ ID NO: 91) |
| anti-VEGF | GSDLDKKLLEAARAGQDDEVRILMANGADVNARDSTGWTPLHLAAP |
| DARPIN | WGHPEIVEVLLKNGADVNAADFQGWTPLHLAAAVGHLEIVEVLLKYG |
| | ADVNAQDKFGKTAFDISIDNGNEDLAEILQKAAGGGSGGGS (SEQ ID |
| | NO: 92) |
| anti-VEGF | QVQLVESGGGLVQPGGSLRLSCAASGYAYDTYYMGWFRQAPGKEREG |
| HuNb22 | VAGITSLVSGVAYYKYYTDSVKGRFTISRDNSKNTVDLQMNSLRAEDT |
| 2MUT | AVYYCAASRSGLRARLLRPELYEYWGQGTLVTVSS (SEQ ID NO: 93) |
| anti-VEGF | QVQLVESGGGLVQPGGSLRLSCVASGDTYSSACMGWFRQAPGKEREG |
| HuNb23 | VATICTSTSMRTRYYADSVKGRFTISRDNSKNTVYLQMNSLRAEDTAV |
| 3MUT | YYCATGHTVGSSWRDPGAWRYWGQGTLVTVSS (SEQ ID NO: 94) |
| anti-VEGF | QVQLVESGGGLVQPGGSLRLSCAASGLSYRPGYMGWFRQAPGKEREG |
| HuNb35 | VAIITTGGVTHYADSVKGRFTISRDNSKNTVYLQMNSLRAEDTAVYYC |
| 4MUT | ALANWVQFPLRVDGYKYWGQGTLVTVSS (SEQ ID NO: 95) |
| Hu_aVEGF_ | QVQLVESGGGLVQPGGSLRLSCAASGRTFSSYSMGWFRQAPGKEREFV |
| VHH_3MUT | AAISKGGYKYDAVSLEGRFTISRDNSKNTVYLQMNSLRPEDTAVYYCA |
| | SSRAYGSSRLRLADTYEYWGQGTLVTVSS (SEQ ID NO: 96) |
| Hu_aVEGF_ | QVQLVESGGGLVQPGGSLRLSCAASGRTFSSYSMGWFRQAPGKEREFV |
| VHH_5MUT | AAISKGGYKYDAVSVKGRFTISRDNSKNTVYLQMNSLRPEDTAVYYCA |
| | SSRAYGSSRLRLADTYEYWGQGTLVTVSS (SEQ ID NO: 97) |
| Hu_aVEGF_ | QVQLVESGGGLVQPGGSLRLSCAASGRTFSSYSMGWFRQAPGKEREFV |
| VHH_6MUT | AAISKGGYKYYAVSVKGRFTISRDNSKNTVYLQMNSLRPEDTAVYYCA |
| | SSRAYGSSRLRLADTYEYWGQGTLVTVSS (SEQ ID NO: 98) |
| blank | SEQ ID NO: 99-100 |
| anti-TNFα | QVQLQESGGGLVQPGGSLRLSCAASGRTFSDHSGYTYTIGWFRQAPGK |
| VHH | EREFVARIYWSSGNTYYADSVKGRFAISRDIAKNTVDLTMNNLEPEDT |
| (human)- | AVYYCAARDGIPTSRSVESYNYWGQGTQVTVSSPSTPPTPSPSTPPGGC |
| rigid | DDDDK (SEQ ID NO: 101) |
| anti-TNFα | QVQLQESGGGLVQPGGSLRLSCAASGRTFSDHSGYTYTIGWFRQAPGK |
| VHH | EREFVARIYWSSGNTYYADSVKGRFAISRDIAKNTVDLTMNNLEPEDT |
| (human)- | AVYYCAARDGIPTSRSVESYNYWGQGTQVTVSSAEAAAKEAAAKEAA |
| aH | AKAGC (SEQ ID NO: 102) |
| anti-TNFα | QVQLQDSGGGLVQAGGSLRLSCAASGGTFSSIIMAWFRQAPGKEREFV |
| VHH | GAVSWSGGTTVYADSVLGRFEISRDSARKSVYLQMNSLKFPEDTAVYY |
| (mouse)- | CAARPYQKYNWASASYNVWGQGTQVTVSSAEAAAKEAAAKEAAAK |
| aH | AGC (SEQ ID NO: 103) |
| anti-TNFα | QVQLQESGGGLVQAGGSLRLSCAASGGTFSSIIMAWFRQAPGKEREFV |
| 3MUT | GAVSWSGGTTVYADSVKGRFTISRDSARKSVYLQMNSLKPEDTAVYY |
| VHH | CAARPYQKYNWASASYNVWGQGTQVTVSSAEAAAKEAAAKEAAAK |
| (mouse)- | AGC (SEQ ID NO: 104) |
| aH | |
| anti-TNFα | CGGGVDNKFNKEVGWAFGEIGALPNLNALQFRAFIISLWDDPSQSANL |
| affibody | LAEAKKLNDAQAPK (SEQ ID NO: 105) |
| anti-IL-1ß | EIVMTQSPSTLSASVGDRVIITCQASQSIDNWLSWYQQKPGKAPKLLIYR |
| scFv-rigid | ASTLASGVPSRFSGSGSGAEFTLTISSLQPDDFATYYCQNTGGGVSIAFG |
| | QGTKLTVLGGGGGSGGGGSGGGGSGGGGSEVQLVESGGGLVQPGGSL |
| | RLSCTASGFSLSSAAMAWVRQAPGKGLEWVGIIYDSASTYYASWAKG |
| | RFTISRDTSKNTVYLQMNSLRAEDTAVYYCARERAIFSGDFVLWGQGT |
| | LVTVSSSPSTPPTPSPSTPPGGC (SEQ ID NO: 106) |
| Hu_aTNFα | QVQLVESGGGLVQPGGSLRLSCAASGGTFSSIIMAWFRQAPGKEREFVG |
| Mu_3MUT | AVSWSGGTTVYADSVKGRFTISRDNSKNTVYLQMNSLRPEDTAVYYC |
| | AARPYQKYNWASASYNVWGQGTLVTVSS (SEQ ID NO: 107) |
| Hu_aTNFα | QVQLVESGGGLVQPGGSLRLSCAASGGTFSSIIMAWFRQAPGKEREFVA |
| Mu_5MUT | AISWSGGTTVYADSVKGRFTISRDNSKNTVYLQMNSLRPEDTAVYYCA |
| | ARPYQKYNWASASYNVWGQGTLVTVSS (SEQ ID NO: 108) |
| Hu_aTNFα | QVQLVESGGGLVQPGGSLRLSCAASGRTFSDHSGYTYTMGWFRQAPG |
| Hu_7MUT | KEREFVARIYWSSGNTYYADSVKGRFTISRDNSKNTVYLQMNSLRPED |
| | TAVYYCAARDGIPTSRSVESYNYWGQGTLVTVSS (SEQ ID NO: 109) |
| blank | SEQ ID NO: 110 |
| Hu_aEGFR_ | EVQLVESGGGLVQPGGSLRLSCAASGRTSRSYGMGWFRQAPGKEREFV |
| 3MUT | AGISWRGDSTGYADSVKGRFTISRDNSKNTVDLQMNSLRPEDTAVYYC |
| | AAAAGSAWYGTLYEYDYWGQGTLVTVSS (SEQ ID NO: 111) |
| Hu_aHer2_ | EVQLVESGGGLVQPGGSLRLSCAASGITFMRYAMGWYRQAPGKQREM |
| 3MUT | VASINSGGTTNYADSVKGRFTISRDNSKNTVYLQMNSLRPEDTAVYYC |
| | NARWVKPQFIDNNYWGQGTLVTVSS (SEQ ID NO: 112) |
| Hu_aPD1_ | EVQLVESGGGLVQPGGSLRLSCAASGSIFSIHAMGWFRQAPGKEREFVA |
| 102C3 | AITWSGGITYYEDSVKGRFTISRDNSKNTVYLQMNSLRPEDTAVYYCA |
| 3MUT | ADRAESSWYDYWGQGTLVTVSS (SEQ ID NO: 113) |
| Hu_aPD1_ | EVQLVESGGGLVQPGGSLRLSCAASGSIASIHAMGWFRQAPGKEREFV |
| 102C12 | AVITWSGGITYYADSVKGRFTISRDNSKNTVYLQMNSLRPEDTAVYYC |
| 3MUT | AGDKHQSSWYDYWGQGTLVTVSS (SEQ ID NO: 114) |
| Hu_aPD1_ | EVQLVESGGGLVQPGGSLRLSCAASGSISSIHAMGWFRQAPGKEREFVA |
| 102E2 | AITWSGGITYYADSVKGRFTISRDNSKNTVYLQMNSLRPEDTAVYYCA |
| 3MUT | ADRAQSSWYDYWGQGTLVTVSS (SEQ ID NO: 115) |
| Hu_aPD1_ | EVQLVESGGGLVQPGGSLRLSCAASGSIFSINAMAWFRQAPGKEREFVA |
| 102E8 | LISWSGGSTYYEDSVKGRFTISRDNSKNTVYLQMNSLRPEDTAVYYCA |
| 3MUT | ADRVDSNWYDYWGQGTLVTVSS (SEQ ID NO: 116) |
| Hu_aPD1_ | EVQLVESGGGLVQPGGSLRLSCAASGRAFSSGTMGWFRQAPGKEREFV |
| 102H12 | ASIPWSGGRTYYADSVKGRFTISRDNSKNTVYLQMNSLRPEDTAVYYC |
| 4MUT | AVKERSTGWDFAWGQGTLVTVSS (SEQ ID NO: 117) |
| Hu | EVQLVESGGGLVQPGGSLRLSCAASGRTGTIYSMAWFRQAPGKEREFL |
| aCaffeine | ATIGWSSGITYYMDSVKGRFTISRDNSKNTVYLQMNSLRPEDTAVYYC |
| 3MUT | AATRAYSVGYDYWGQGTLVTVSS (SEQ ID NO: 118) |
| Hu_aEGFR_ | QVQLVESGGGLVQPGGSLRLSCAASGRTSRSYGMGWFRQAPGKEREF |
| 5MUT | VAGISWRGDSTGYADSVKGRFTISRDNSKNTVYLQMNSLRPEDTAVYY |
| | CAAAAGSAWYGTLYEYDYWGQGTLVTVSS (SEQ ID NO: 119) |
| aAng2_D4 | EVQLVESGGGLVQAGGSLRLSCVASGRIFTNTAMGWYRQAPGKWREL |
| | VATIYSGGSTKYIDSVKGRFIISRDNTRNTVHLQMNSLKPEDTAVYYCN |
| | TVGAGSYWGQGAQVTVSS (SEQ ID NO: 120) |
| Hu_aAng2_ | QVQLVESGGGLVQPGGSLRLSCAASGRIFTNTAMGWYRQAPGKWREL |
| D4 3MUT | VATIYSGGSTKYIDSVKGRFTISRDNSKNTVYLQMNSLRPEDTAVYYCA |
| | TVGAGSYWGQGTLVTVSS (SEQ ID NO: 121) |
| aAn2_H4 | EVQLVESGGDLVQAGGSLRLSCAASGFTFGDFTIGWFRQAPGKEREGV |
| | SCINTGDGSTNYAESVKGRFTISSDNAKNTVYLQMNSLKPEDTAVYYC |
| | ALDQAPMWSSWSAPYEYDYWGQGTQVTVSS (SEQ ID NO: 122) |
| Hu_aAng2_ | QVQLVESGGGLVQPGGSLRLSCAASGFTFGDFTMGWFRQAPGKEREG |
| H4_6MUT | VAAINTGDGSTNYADSVKGRFTISRDNSKNTVYLQMNSLRPEDTAVYY |
| | CALDQAPMWSSWSAPYEYDYWGQGTLVTVSS (SEQ ID NO: 123) |
| aAng2_BI0027 | EVQLVESGGGLVQAGGSLRLSCAASGFTLDDYAIGWFRQAPGKEREGV |
| | SSIRDNDGSTYYADSVKGRFTISSDNDKNTVYLQMNSLKPEDTAVYYC |
| | AAVPAGRLRFGEQWYPLYEYDAWGQGTLVTVSS (SEQ ID NO: 124) |
| Hu_aAng2_ | QVQLVESGGGLVQPGGSLRLSCAASGFTLDDYAMGWFRQAPGKEREG |
| 0027_2MUT | VASIRDNDGSTYYADSVKGRFTISRDNSKNTVYLQMNSLRPEDTAVYY |
| | CAAVPAGRLRFGEQWYPLYEYDAWGQGTLVTVSS (SEQ ID NO: 125) |
| aEpCam_Nb4 | QVQLVQSGGGSVQGGASLRLSCAASGGERNNYCVAWFRQAPGKEREV |
| | AAISRAASGAQTTTKYYVDSVKGRFTISQDTKNTATVYLQMNSHKPED |
| | TAYCTAKAKIYPPQCTGISRTIDYRGQGTQVTVSS (SEQ ID NO: 126) |
| Hu_aEpCAM_ | QVQLVESGGGLVQPGGSLRLSCAASGGERNNYCMGWFRQAPGKEREV |
| Nb4_4 | AAISRAASGAQTTTKYYVDSVKGRFTISRDNSKNTVYLQMNSLRPEDT |
| MUT | AYCAAKAKIYPPQCTGISRTIDYRGQGTLVTVSS (SEQ ID NO: 127) |
| aEpCam_Nb4 | QVQLVQSGGGLVQGGASLRLSCAASSGRAGLIHVAWFRAKNTLYRQV |
| | AAISSDDTGFATLYYAVDSVKGRFTISQDTESTKYSYLQMNSLKPEDTI |
| | YCTAKLFSKTSYQKFIYKFNYWGQGTQVTVSS (SEQ ID NO: 128) |
| Hu_aEpCAM_ | QVQLVESGGGLVQPGGSLRLSCAASGGRAGLIHMGWFRQAPGKYREV |
| Nb5_11 | AAISSDDTGFATLYYAVDSVKGRFTISRDNSKNTVYLQMNSLRPEDTA |
| MUT | YCTAKLFSKTSYQKFIYKFNYWGQGTLVTVSS (SEQ ID NO: 129) |
| aEpCAM_ | QVQQVQSGGGSVQAGASLRLSCAASSGLQQAIYVAWFRQAVGKEREV |
| Nb22 | AAIGYWYWYHIQVYKYYVDSVKDRFTISGDTKSTKTYLQMNSLKNED |
| | TAYYCAKIHHLPRQRQEHGIFDYRGQGTQVTVSS (SEQ ID NO: 130) |
| Hu_aEpCAM_ | QVQLVESGGGLVQPGGSLRLSCAASGGLQQAIYMGWFRQAPGKEREV |
| Nb22_6 | AAIGYWYWYHIQVYKYYVDSVKDRFTISRDNSKNTVYLQMNSLRPED |
| MUT | TAYYCAKIHHLPRQRQEHGIFDYRGQGTLVTVSS (SEQ ID NO: 131) |
| blank | SEQ ID NO: 132-140 |
| Hu2H10 | QVQLVESGGGLVQPGGSLRLSCLASGRRFSIEAMGWYRQAPGKQRELV |
| 5MUT | ALIDSGGSTDYVDSAKGRFTISRDNSKNTVYLQMNSLRAEDTAVYYCN |
| CYS | VIGSSWYGRGLDYWGQGTLVTVSSC (SEQ ID NO: 141) |
| Hu2H10 | QVQLVESGGGLVQPGGSLRLSCLASGRRFSIEAMGWYRQAPGKQRELV |
| 5MUT | AIIDSGGSTDYVDSAKGRFTISRDNSKNTVYLQMNSLRAEDTAVYYCN |
| aH_CYS | VIGSSWYGRGLDYWGQGTLVTVSSAEAAAKEAAAKEAAAKAGC (SEQ |
| | ID NO: 142) |
| Hu2H10 | QVQLVESGGGLVQPGGSLRLSCLASGRRFSIEAMGWYRQAPGKQRELV |
| 5MUT | ALIDSGGSTDYVDSAKGRFTISRDNSKNTVYLQMNSLKPEDTAVYYCN |
| R86K | VIGSSWYGRGLDYWGQGTLVTVSSAEAAAKEAAAKEAAAKAGC (SEQ |
| A87P | ID NO: 143) |
| aH_CYS | |
| Hu2H10 | QVQLVESGGGLVQPGGSLRLSCLASGRRFSIEAMGWYRQAPGKQRELV |
| 5MUT | AIIDSGGSTDYVDSAKGRFTISRDNSKNTVYLQMNSLRAEDTAVYYCN |
| L115Q | VIGSSWYGRGLDYWGQGTQVTVSSAEAAAKEAAAKEAAAKAGC |
| aH_CYS | (SEQ ID NO: 144) |
| HuNb42 | QVQLVESGGGLVQPGGSLRLSCAASGFAYSTYSMGWFRQAPGKEREA |
| A88P | VATINSGTFRLWYTDSVKGRFTISRDNSKNTLYLQMNSLRPEDTAVYY |
| aH_CYS | CAARAWSPYSSTVDAGDFRYWGQGTLVTVSSAEAAAKEAAAKEAAA |
| | KAGC (SEQ ID NO: 145) |
| HuNb42 | QVQLVESGGGLVQPGGSLRLSCAASGFAYSTYSMGWFRQAPGKEREA |
| T61A A88P | VATINSGTFRLWYADSVKGRFTISRDNSKNTLYLQMNSLRPEDTAVYY |
| aH_CYS | CAARAWSPYSSTVDAGDFRYWGQGTLVTVSSAEAAAKEAAAKEAAA |
| | KAGC (SEQ ID NO: 146) |
| HuNb42 | QVQLVESGGGLVQPGGSLRLSCAASGFAYSTYSMGWFRQAPGKEREA |
| A88P | VATINSGTFRLWYTDSVKGRFTISRDNSKNTLYLQMNSLRPEDTAVYY |
| L115Q | CAARAWSPYSSTVDAGDFRYWGQGTQVTVSSAEAAAKEAAAKEAAA |
| aH_CYS | KAGC (SEQ ID NO: 147) |
| anti-TNFα | CGGGVDNKFNKEVGWAFGEIGALPNLNALQFRAFIISLWDDPSQSANL |
| affibody | LAEAKKLNDAQAPKGGG (SEQ ID NO: 148) |
| Gly | |
| Hu_aTNFα | QVQLVESGGGLVQPGGSLRLSCAASGGTFSSIIMAWFRQAPGKEREFVG |
| Mu_3MUT | AVSWSGGTTVYADSVKGRFTISRDNSKNTVYLQMNSLRPEDTAVYYC |
| aH_CYS | AARPYQKYNWASASYNVWGQGTLVTVSSAEAAAKEAAAKEAAAKA |
| | GC (SEQ ID NO: 149) |
| Hu_aTNFα | QVQLVESGGGLVQPGGSLRLSCAASGGTFSSIIMAWFRQAPGKEREFVA |
| Mu_5MUT | AISWSGGTTVYADSVKGRFTISRDNSKNTVYLQMNSLRPEDTAVYYCA |
| aH_CYS | ARPYQKYNWASASYNVWGQGTLVTVSSAEAAAKEAAAKEAAAKAGC |
| | (SEQ ID NO: 150) |
| Hu_aTNFα | QVQLVESGGGLVQPGGSLRLSCAASGRTFSDHSGYTYTMGWFRQAPG |
| Hu_7MUT | KEREFVARIYWSSGNTYYADSVKGRFTISRDNSKNTVYLQMNSLRPED |
| aH_CYS | TAVYYCAARDGIPTSRSVESYNYWGQGTLVTVSSAEAAAKEAAAKEA |
| | AAKAGC (SEQ ID NO: 151) |
| IL- | APTSSSTKKTQLQLEHLLLDLQMILNGINNYKNPKLTRMLTFKFYMPKK |
| 2_C125S | ATELKHLQCLEEELKPLEEVLNLAQSKNFHLRPRDLISNINVIVLELKGS |
| aH_CYS | ETTFMCEYADETATIVEFLNRWITFSQSIISTLTAEAAAKEAAAKEAAAK |
| | AGC (SEQ ID NO: 152) |
| IL- | NWVNVISDLKKIEDLIQSMHIDATLYTESDVHPSCKVTAMQCFLSELQV |
| 15_5MUT | ISLESGDASIHDTVENLTILANNSLSSNGYVTESGCKECEELEAKNIKEFL |
| aH_CYS | QSFVHIVQMFINTSAEAAAKEAAAKEAAAKAGC (SEQ ID NO: 153) |
| aTNFα_DARPin | DLGKKLLEVARAGQDDEVRILMANGADVNAADHQSFTPLHLYAIFGH |
| G3S_CYS | LEIVEVLLKNGADVNASDWHGNTPLHLAAWIGHLEIVEVLLKYGADV |
| | NATDHSGSTPLHLAATLGHLEIVEVLLKYGADVNAQDKFGKTAFDISID |
| | NGNEDLAEILQKAAGGGSGGGSC (SEQ ID NO: 154) |
| aPDL1_ Nb1 | QVQLQESGGGLVQAGGSLRLSCATSGSIFSIISMGWYRQAPGKQRELVA |
| | LVFRGGSTVYADSVKGRFTISGDIAKNTVYLQMDSLKPEDTAVYYCNV |
| | KSIGTAQYWGQGTQVTVSS (SEQ ID NO: 155) |
| Hu_aPDL1_ | QVQLVESGGGLVQPGGSLRLSCAASGSIFSIISMGWYRQAPGKQRELVA |
| Nb1_4MUT | LIFRGGSTVYADSVKGRFTISRDNSKNTVYLQMNSLRPEDTAVYYCAV |
| | KSIGTAQYWGQGTLVTVSS (SEQ ID NO: 156) |
| aPDL1_Nb | EVOLVESGGGLVQAGGSLRLSCAASGVDASNSAMGWYRQAPGKQRE |
| 104E10 | WVARITGGGLIAYTDSVKGRFTISRDNAKSTVYLQMNSLEPEDTAVYY |
| | CNTINSRDWGQGTQVTVSS (SEQ ID NO: 157) |
| Hu_aPDL1_ | QVQLVESGGGLVQPGGSLRLSCAASGVDASNSAMGWYRQAPGKQRE |
| Nb104E10_ | WVARITGGGLIAYTDSVKGRFTISRDNSKSTVYLQMNSLRPEDTAVYY |
| 1MUT | CATINSRDWGQGTLVTVSS (SEQ ID NO: 158) |
| aPDL1_NbG5 | QVQLQESGGGLVQPGGSLRLSCAASGSFFRTYIVSWYRQAPGKRREAV |
| | AVMSNSGNTNYADSVKGRFTISRDNAVNTVYLQMNSLKPEDTAVYYC |
| | NLLKVTVVPPANYWGQGTQVTVSS (SEQ ID NO: 159) |
| Hu_aPDL1_ | QVQLVESGGGLVQPGGSLRLSCAASGSFFRTYIMGWYRQAPGKRREAV |
| NbG5_4MUT | AVISNSGNTNYADSVKGRFTISRDNSKNTVYLQMNSLRPEDTAVYYCA |
| | LLKVTVVPPANYWGQGTLVTVSS (SEQ ID NO: 160) |
| aPDL2_103 | EVQLVESGGGLVQAGGSLRLSCAASGSTFSNYVSNYAMGWGRQAPGT |
| E4 | QRELVASISNGDTTNYADSVKGRFTISRDNAKNTVYLQMNSLKPEDTA |
| | VYYCFEHQVAGLTWGQGTQVTVSS (SEQ ID NO: 161) |
| Hu_aPDL2_ | QVQLVESGGGLVQPGGSLRLSCAASGSTFSNYVSNYAMGWGRQAPGK |
| 103E4_2MUT | QRELVASISNGDTTNYADSVKGRFTISRDNSKNTVYLQMNSLRPEDTA |
| | VYYCAEHQVAGLTWGQGTLVTVSS (SEQ ID NO: 162) |
| aPDL2_103 | EVQLVESGGGLVQTGGSLRLSCAASGFTLDYYGIGWFRQAPGKEREGV |
| E5 | SFISGSDGSTYYAESVKGRFTISRDKAKNTVYLQMNSLKPEDTAVYYCA |
| | ADPWGPPSIATMTSYEYKHWGQGTQVTVSS (SEQ ID NO: 163) |
| Hu_aPDL2_ | QVQLVESGGGLVQPGGSLRLSCAASGFTLDYYGMGWFRQAPGKEREG |
| 103E5_4MUT | VAFISGSDGSTYYAESVKGRFTISRDNSKNTVYLQMNSLRPEDTAVYYC |
| | AADPWGPPSIATMTSYEYKHWGQGTLVTVSS (SEQ ID NO: 164) |
| aPDL2_Nb | EVQLVESGGGLVQAGGSLRLSCAASGRTFSSGTMGWFRRAPGKEREFV |
| 103F10 | ASIPWSGGRTYYADSVKDRFTISRDNAQNTVFLQMNSLKPEDTAVYYC |
| | AFKERSTGWDFASWGQGIQVTVSS (SEQ ID NO: 165) |
| Hu_aPDL2_ | QVQLVESGGGLVQPGGSLRLSCAASGRTFSSGTMGWFRQAPGKEREFV |
| Nb103F10_ | ASIPWSGGRTYYADSVKGRFTISRDNSKNTVYLQMNSLRPEDTAVYYC |
| 5MUT | AFKERSTGWDFASWGQGTLVTVSS (SEQ ID NO: 166) |
| Hu_aIL- | QVQLVESGGGLVQPGGSLRLSCAASGGYYYSSFYWGWFRQAPGQGLE |
| 1B_5C_5MUT | AVATIPYYYTYYTDSVKGRFTISRDNSKNTVYLQMNSLRPEDTAVYYC |
| | ASYNIHNFWGQGTLVTVSS (SEQ ID NO: 167) |
| Hu_aIL- | QVQLVESGGGLVQPGGSLRLSCAASGSYSYSYSWGWFRQAPGQGLEA |
| 1B_6C_3MUT | VAAIVYYYTYYADSVKGRFTISRDNSKNTVYLQMNSLRPEDTAVYYCA |
| | SYAPFPFWGQGTLVTVSS (SEQ ID NO: 168) |
| Hu_aIL- | QVQLVESGGGLVQPGGSLRLSCAASGGYSYYSWGWFRQAPGQGLEAV |
| 1B_11H_3MUT | ASIAYYYTYYADSVKGRFTISRDNSKNTVYLQMNSLRPEDTAVYYCAY |
| | SDIISYWGQGTLVTVSS (SEQ ID NO: 169) |
| Hu_aIL- | QVQLVESGGGLVQPGGSLRLSCAASGSYYSSSYGGWFRQAPGQGLEA |
| 1B_12D_2MUT | VATIPYYTSYADSVKGRFTISRDNSKNTVYLQMNSLRPEDTAVYYCASF |
| | FPFFYWGQGTLVTVSS (SEQ ID NO: 170) |

What is claimed is:

1. A conjugate that is a random polymer of Formula III:
(X—Y—Z¹)ₙ—(Z²)ₚ—(Z³)_{q} (III),
having a molecular weight of from about 0.1 MDa to about 3 MDa;
wherein
each X is independently a peptide having a molecular weight of from about 5 kDa to about 200 kDa;
each Y is an organic linker;
each X—Y-Z¹ moiety has the structure: 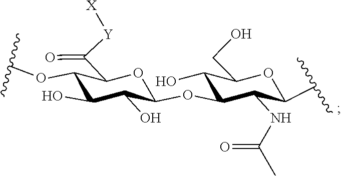
each Z² has the structure: 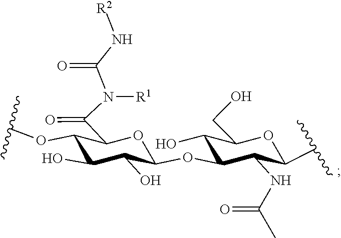
each Z³ independently has the structure: 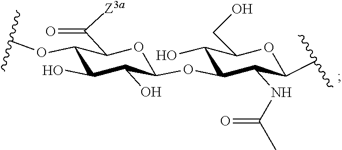
each R¹ and R² is independently C₁-C₆ alkyl, —(C₁-C₆ alkyl)-NR³R⁴, or C₅-C₈ cycloalkyl;
each R3 and R⁴ is independently H or C₁-C₆ alkyl;
each Z^{3a} is independently OH or Y′;
each Y′ is an unreacted organic linker;
subscript n is an integer of from 1 to 1500 and less than about 15% of the sum of subscripts n, p, and q;
subscript p is an integer of from 0 to 1000 and less than about 10% of the sum of subscripts n, p, and q; and
subscript q is an integer of from 100 to 10000.

2. The conjugate of claim 1, wherein the conjugate has the structure of Formula IIIa:
(X¹—X²—Y—Z¹)ₙ(Z²)ₚ—(Z³)_{q} (IIIa),
wherein
each X¹ comprises a peptide having Formula (I):
FR1-CDR1-FR2-CDR2-FR3-CDR3-FR4 (I),
CDR1, CDR2, and CDR3 are each independently complementarity-determining regions;
FR1 has an amino acid sequence comprising
(SEQ ID NO: 1)
X¹⁰VQLX¹¹EX¹²GGGX¹³X¹⁴QX¹⁵GX¹⁶SLRLSCX¹⁷X¹⁸SG,
wherein
X¹⁰ is Q, E, or D,
X¹¹ is V, Q, A, or E,
X¹² is S or T,
X¹³ is L, S, or V,
X¹⁴ is V or A,
X¹⁵ is P, A, or T,
X¹⁶ is G, D, or R,
X¹⁷ is A, V, T, or E, and
X¹⁸ is A or V;
FR2 has an amino acid sequence comprising
(SEQ ID NO: 2)
X²⁰X²¹WX²²RQX²³PGKX²⁴X²⁵EX²⁶VX²⁷X²⁸I,
wherein
X²⁰ is M, I, V, or L,
X²¹ is G, S, or A,
X²² is F, Y, or V,
X²³ is A, V, P, or T,
X²⁴ is E, G, A, or Q,
X²⁵ is R or L,
X²⁶ is F, G, W, or L,
X²⁷ is A, G, or S, and
X²⁸ is A, S, or G;
FR3 has an amino acid sequence comprising
(SEQ ID NO: 3)
YX³⁰DSVKGRFTISX³¹DX³²X³³KX³⁴X³⁵VX³⁶LQMX³⁷X³⁸LRX^{39ª}EDTAX^{39b}
YYCAA,
wherein
X³⁰ is A, G, S, or T,
X³¹ is R or Q,
X³² is N, S, or D,
X³³ is S, A, or D,
X³⁴ is N or K,
X³⁵ is T or M,
X³⁶ is Y, D, or S,
X³⁷ is N or D,
X³⁸ is S or N,
X³⁹a is P or A, and
X³⁹b is V, M, L, or I;
and
FR4 has an amino acid sequence comprising
(SEQ ID NO: 4)
YWGX⁴⁰GTX⁴¹VTVSS,
wherein
X⁴⁰ is Q or K, and
X⁴¹ is L or Q; and
each X² is a peptide linker that comprises an alpha-helix.

3. The conjugate of claim 2, wherein X²⁷ is A.

4. The conjugate of any one of claims 2 to 3, wherein X^{39a} is P.

5. The conjugate of any one of claims 2 to 4, wherein
FR1 has an amino acid sequence comprising
(SEQ ID NO: 5)
QVQLVESGGGLVQPGGSLRLSCAASG.

6. The conjugate of any one of claims 2 to 5, wherein
FR2 has an amino acid sequence comprising
(SEQ ID NO: 6)
MGWFRQAPGKEREFVAAI.

7. The conjugate of any one of claims 2 to 6, wherein
FR3 has an amino acid sequence comprising
(SEQ ID NO: 7)
YADSVKGRFTISRDNSKNTVYLQMNSLRPEDTAVYYCAA.

8. The conjugate of any one of claims 2 to 7, wherein FR4 has an amino acid sequence comprising YWGQGTLVTVSS (SEQ ID NO: 8).

9. The conjugate of any one of claims 2 to 8, wherein CDR1, CDR2, and CDR3 are each complementarity-determining regions from an antibody or a cytokine.

10. The conjugate of claim 9, wherein the antibody is a monoclonal IgG, an IgG fragment, single chain scFv, single-domain heavy-chain VHH, adnectin, affibody, anticalin, DARPin, or an engineered Kunitz-type inhibitor.

11. The conjugate of claim 9 or 10, wherein the complementarity-determining regions are each specific to vascular endothelial growth factor (VEGF), tumor necrosis factor-alpha (TNF-α), programmed cell death protein 1 (PD-1), programmed death ligand-1 (PD-L1), cytotoxic T-lymphocyte-associated protein 4 (CTLA4), cluster of differentiation 40 (CD40), cluster of differentiation 134 (CD134), cluster of differentiation 137 (CD137), glucocorticoid-induced tumor necrosis factor receptor-related protein (GITR), V-domain immunoglobulin suppressor of T-cell activation (VISTA), T cell immunoglobulin and mucin domain-containing protein 3 (TIM-3), lymphocyte activating 3 (LAG3), interleukin-1-beta (IL-1R), interleukin-6 (IL-6), interleukin-10 (IL-10), interleukin-12 (IL-12), or interleukin-15 (IL-15).

12. The conjugate of claim 9 or 10, wherein the complementarity-determining regions are each specific to vascular endothelial growth factor (VEGF).

13. The conjugate of any one of claims 2 to 12, wherein the peptide consists of Formula I.

14. The conjugate of any one of claims 2 to 13, having one or more of the following:
(a) a CDR1 of 7 amino acids in length;
(b) a CDR2 of 7 or 8 amino acids in length; and/or
(c) a CDR3 of 9 to 16 amino acids in length.

15. The peptide of any one of claims 2 to 14, wherein:
(a) CDR1 has an amino acid sequence comprising FAYSTYS (SEQ ID NO: 9),
CDR2 has an amino acid sequence comprising NSGTFRLW (SEQ ID NO: 10), and
CDR3 has an amino acid sequence comprising RAWSPYSSTVDAGDFR (SEQ ID NO: 11); or
(b) CDR1 has an amino acid sequence comprising RRFSIEA (SEQ ID NO: 12),
CDR2 has an amino acid sequence comprising DSGGSTD (SEQ ID NO: 13), and
CDR3 has an amino acid sequence comprising IGGSWYGRGLD (SEQ ID NO: 14); or
(c) CDR1 has an amino acid sequence comprising GTFSSII (SEQ ID NO: 15),
CDR2 has an amino acid sequence comprising SWSGGTTV (SEQ ID NO: 16), and
CDR3 has an amino acid sequence comprising RPYQKYNWASASYNV (SEQ ID NO: 17); or
(d) CDR1 has an amino acid sequence comprising GGSDAGT (SEQ ID NO: 18),
CDR2 has an amino acid sequence comprising SWAGTAWR (SEQ ID NO: 19), and
CDR3 has an amino acid sequence comprising LGSYEMDHH (SEQ ID NO: 20).

16. The conjugate of claim 2, wherein each X¹ is a peptide having an amino acid sequence comprising any one of SEQ ID NOS: 51-58, 61-73, 81-85, 91-95, 101-106, and 111-118.

17. The conjugate of any one of claims 2 to 16, wherein
each X² is a peptide linker having an amino acid sequence comprising:
(SEQ ID NO: 21)
AEAAAKEAAAKEAAAKAGC,
(SEQ ID NO: 22)
AEEEKRKAEEEKRKAEEEAGC,
(SEQ ID NO: 23)
AEEEKRKAEEEKRKAEEEKRKAEEEAGC,
(SEQ ID NO: 24)
AEEEEKKKKEEEEKKKKAGC,
(SEQ ID NO: 25)
AEAAAKEAAAKAGC,
(SEQ ID NO: 26)
PSRLEEELRRRLTEGC,
or
(SEQ ID NO: 27)
AEEEEKKKQQEEEAERLRRIQEEMEKERKRREEDEERRRKEEEERRM
KLEMEAKRKQEEEERKKREDDEKRKKKAGC.

18. The conjugate of any one of claims 1 to 17, wherein the organic linker has the structure: 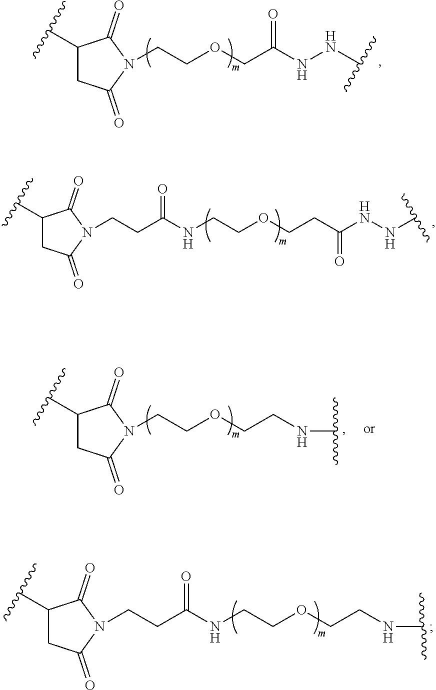
and
subscript m is an integer of from 1 to 300.

19. The conjugate of any one of claims 1 to 18, wherein the organic linker has the structure:

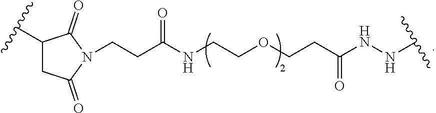

20. The conjugate of any one of claims 1 to 19, wherein the random polymer of Formula III has a molecular weight of from about 0.4 MDa to about 2 MDa.

21. The conjugate of any one of claims 1 to 20, wherein the random polymer of Formula III has a molecular weight of from about 0.7 MDa to about 1.5 MDa.

22. The conjugate of any one of claims 1 to 21, wherein the random polymer of Formula III has a molecular weight of about 0.8 MDa.

23. The conjugate of any one of claims 1 to 22, wherein each R¹ and R² is independently C₁-C₃ alkyl or —(C₁-C₃ alkyl)-NR³R⁴.

24. The conjugate of any one of claims 1 to 23, wherein each R³ and R⁴ is independently C₁-C₃ alkyl.

25. The conjugate of any one of claims 1 to 24, wherein
subscript n is an integer of from 1 to 1500 and less than about 15% of the sum of subscripts n, p, and q;
subscript p is an integer of from 1 to 1000 and less than about 10% of the sum of subscripts n, p, and q; and
subscript q is an integer of from 100 to 10000.

26. The conjugate of any one of claims 1 to 25, wherein
subscript n is an integer of from 1 to 1000 and less than about 10% of the sum of subscripts n, p, and q;
subscript p is an integer of from 1 to 800 and less than about 8% of the sum of subscripts n, p, and q; and
subscript q is an integer of from 100 to 10000.

27. The conjugate of any one of claims 1 to 26, wherein
subscript n is an integer of from 10 to 450 and less than about 15% of the sum of subscripts n, p, and q;
subscript p is an integer of from 1 to 300 and less than about 10% of the sum of subscripts n, p, and q; and
subscript q is an integer of from 1000 to 3000

28. The conjugate of any one of claims 1 to 27, wherein
subscript n is an integer of from 10 to 300 and less than about 10% of the sum of subscripts n, p, and q;
subscript p is an integer of from 1 to 240 and less than about 8% of the sum of subscripts n, p, and q; and
subscript q is an integer of from 1000 to 3000.

29. The conjugate of any one of claims 1 to 28, wherein
subscript n is an integer of from 10 to 300 and less than about 10% of the sum of subscripts n, p, and q;
subscript p is an integer of from 1 to 60 and less than about 2% of the sum of subscripts n, p, and q; and
subscript q is an integer of from 1000 to 3000.

30. The conjugate of any one of claims 1 to 29, wherein
subscript n is an integer of from 10 to 300 and less than about 10% of the sum of subscripts n, p, and q;
subscript p is an integer of from 1 to 30 and less than about 1% of the sum of subscripts n, p, and q; and
subscript q is an integer of from 1000 to 3000.

31. The conjugate of any one of claims 1 to 30, wherein
subscript n is an integer of from 10 to 300 and less than about 10% of the sum of subscripts n, p, and q;
subscript p is an integer of from 1 to 15 and less than about 0.5% of the sum of subscripts n, p, and q; and
subscript q is an integer of from 1000 to 3000.

32. A conjugate that is a random polymer of Formula IIIa:
(X¹—X²—Y—Z¹)ₙ—(Z²)ₚ—(Z³)_{q} (IIIa),
having a molecular weight of about 0.8 MDa;
wherein
each X¹ is a peptide having an amino acid sequence comprising SEQ ID NO: 55;
each X² is a peptide linker having an amino acid sequence comprising
(SEQ ID NO: 21)
AEAAAKEAAAKEAAAKAGC;
each Y is an organic linker having the structure: 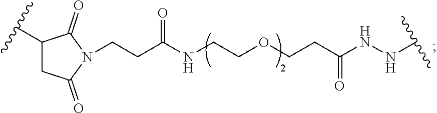
each X¹—X²—Y—Z¹ moiety has the structure: 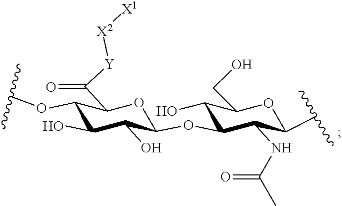
each Z² has the structure: 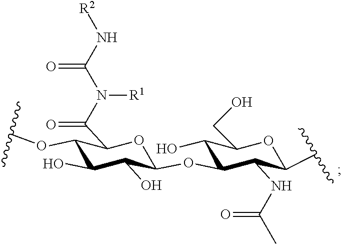
each Z³ independently has the structure: 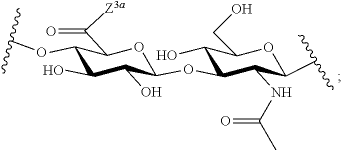
each Z^{3a} is independently OH or Y′;
each Y′ has the structure: 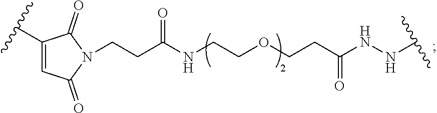
each R¹ and R² is ethyl or —(CH₂)₃—NMe₂;
subscript n is an integer of from 10 to 300 and less than about 10% of the sum of subscripts n, p, and q;
subscript p is an integer of from 1 to 15 and less than about 0.5% of the sum of subscripts n, p, and q; and
subscript q is an integer of from 1000 to 3000.

33. A conjugate that is a random polymer of Formula IIIa:
(X¹—X²—Y—Z¹)ₙ—(Z²)ₚ—(Z³)_{q} (IIIa),
having a molecular weight of about 0.8 MDa;
wherein
each X¹ is a peptide having an anti-VEGF amino acid sequence comprising SEQ ID NO: 67;
each X² is a peptide linker having an amino acid sequence comprising
(SEQ ID NO: 21)
AEAAAKEAAAKEAAAKAGC;
each Y is an organic linker having the structure: 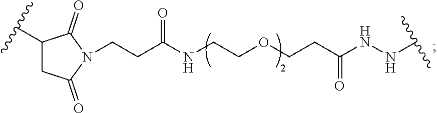
each X¹—X²—Y—Z¹ moiety has the structure: 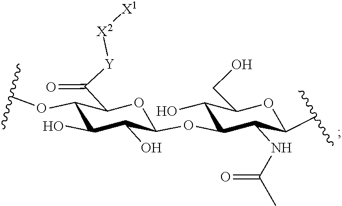
each Z² has the structure: 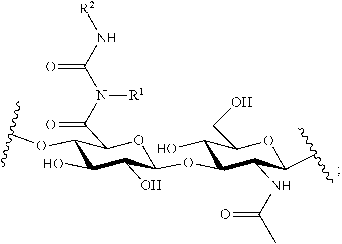
each Z³ independently has the structure: 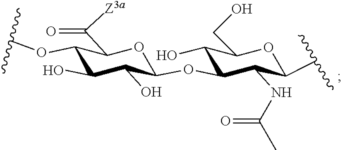
each Z^{3a} is independently OH or Y′;
each Y′ has the structure: 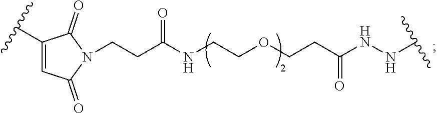
each R¹ and R² is ethyl or —(CH₂)₃—NMe₂;
subscript n is an integer of from 10 to 300 and less than about 10% of the sum of subscripts n, p, and q;
subscript p is an integer of from 1 to 15 and less than about 0.5% of the sum of subscripts n, p, and q; and
subscript q is an integer of from 1000 to 3000.

34. A pharmaceutical composition comprising a conjugate of any one of claims 1 to 33, or a pharmaceutically acceptable salt thereof, and a pharmaceutically acceptable excipient.

35. A method of treating an ocular disorder in a subject in need thereof, comprising administering to the subject a conjugate of any one of claims 1 to 33.

36. The method of claim 35, comprising intravitreally administering the conjugate.

37. The method of claim 35 or 36, comprising administering the conjugate every month, every two months, or every three months.

38. The method of any one of claims 35 to 37, wherein the vitreous half-life of the conjugate is at least 5-fold greater than the half-life of the unconjugated peptide.

39. The method of any one of claims 35 to 38, wherein the ocular disorder is uveitis, macular degeneration, choroidal neovascularization, retinal neovascularization, proliferative vitreoretinopathy, glaucoma, or ocular inflammation.

40. A method of treating a disease or disorder in an articular joint in a subject in need thereof, comprising administering to the subject a conjugate of any one of claims 1 to 33.

41. The method of claim 40, comprising intraarticularly administering the conjugate.

42. The method of claim 40 or 41, comprising administering the conjugate every month, every two months, or every three months.

43. The method of any one of claims 40 to 42, wherein the intraarticular half-life of the conjugate is at least 5-fold greater than the half-life of the unconjugated peptide.

44. The method of any one of claims 40 to 43, wherein the disease or disorder is rheumatoid arthritis, wear-related osteoarthritis, age-related osteoarthritis, post-traumatic osteoarthritis, psoriatic arthritis, and aseptic implant loosening, joint effusion, ankylosing spondylitis, bursitis, gout, reactive arthritis, synovitis, or avascular necrosis.

45. A method of preparing a conjugate of any one of claims 1 to 33, the method comprising:
(a) forming a first reaction mixture comprising a hyaluronic acid polymer having a molecular weight of from about 0.1 MDa to about 3 MDa, from about 0.1 to about 2 equivalents coupling agent per hyaluronic acid monomer, and an organic linker agent of formula H₂N—R^{Y},
wherein
R^{Y} is 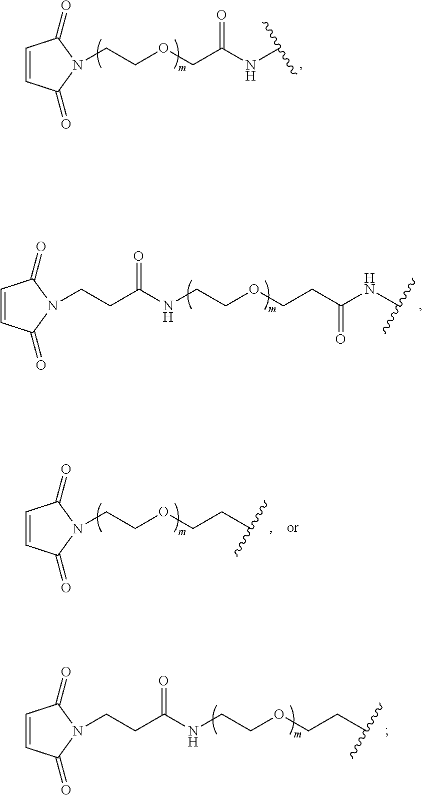
subscript m is an integer of from 1 to 300;
thereby forming an intermediate polymer having a plurality of monomers of Formula IV: 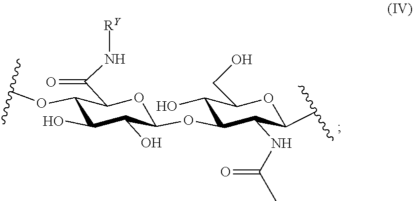
and
(b) forming a second reaction mixture comprising the intermediate polymer and a peptide having a molecular weight of from about 5 kDa to about 200 kDa, wherein the peptide comprises one or more —SH;
thereby preparing the conjugate.

46. The method of claim 45, wherein the hyaluronic acid polymer has a molecular weight of from about 0.4 MDa to about 2 MDa.

47. The method of claim 45, wherein the hyaluronic acid polymer has a molecular weight of from about 0.7 MDa to about 1.5 MDa.

48. The method of claim 45, wherein the hyaluronic acid polymer has a molecular weight of about 0.8 MDa.

49. The method of any one of claims 45 to 48, wherein the first reaction mixture comprises from about 0.2 to about 1.5 equivalents coupling agent per hyaluronic acid monomer.

50. The method of any one of claims 45 to 48, wherein the first reaction mixture comprises from about 0.2 to about 1 equivalent coupling agent per hyaluronic acid monomer.

51. The method of any one of claims 45 to 50, wherein the coupling agent comprises a carbodiimide.

52. The method of any one of claims 45 to 51, wherein the coupling agent is 1-ethyl-3-(3-dimethylaminopropyl)carbodiimide, 1,3-diisopropylcarbodiimide, or dicyclohexyl carbodiimide, or a salt thereof.

53. The method of any one of claims 45 to 52, wherein R^{Y} is

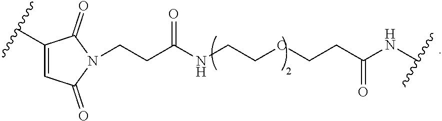

54. The method of any one of claims 45 to 53, wherein the first reaction mixture comprises from about 0.2 to about 6 equivalents of the organic linker agent per hyaluronic acid monomer.

55. The method of any one of claims 45 to 54, wherein the first reaction mixture comprises a catalyst.

56. The method of claim 55, wherein the catalyst is ethyl 2-cyano-2-(hydroxyimino)acetate (Oxyma), hydroxybenzotriazole, N-hydroxysuccinimide (NHS), N-hydroxysulfosuccinimide (sulfo-NHS), or 1-hydroxy-7-azabenzotriazole, or a salt thereof.

57. The method of any one of claims 45 to 56, wherein the second reaction mixture comprises from about 0.5 to about 1.5 equivalents peptide per organic linker.
